(19) United States
(12) Patent Application Publication
(10) Pub. No.: US 2024/0260290 A1
(43) Pub. Date: Aug. 1, 2024

(54) COMPOSITION, LIGHT-EMITTING DEVICE, AND ELECTRONIC APPARATUS INCLUDING THE SAME

(71) Applicant: Samsung Display Co., Ltd., Yongin-si (KR)

(72) Inventors: Jiyong Choi, Yongin-si (KR); Heechoon Ahn, Yongin-si (KR); Hyunah Um, Yongin-si (KR); Yeseul Lee, Yongin-si (KR); Hyoyoung Lee, Yongin-si (KR); Seowon Cho, Yongin-si (KR); Moran Ha, Yongin-si (KR)

(21) Appl. No.: 18/406,939

(22) Filed: Jan. 8, 2024

(30) Foreign Application Priority Data
Jan. 9, 2023 (KR) 10-2023-0003028

(51) Int. Cl. H10K 50/11; C07D 403/14; C07D 405/14; H10K 85/30; H10K 85/60

(52) U.S. Cl. CPC H10K 50/11; C07D 403/14; C07D 405/14; H10K 85/346; H10K 85/6572; H10K 85/658

(57) ABSTRACT

A composition includes a first compound represented by Formula 1 and a second compound represented by Formula 2. A light-emitting device includes the two compounds, a first electrode, a second electrode facing the first electrode, and an interlayer including an emission layer, and an electronic apparatus and electronic equipment each including the light-emitting device. The triplet (T1) energy levels of the two compounds are configured such that the luminescence efficiency and lifetime of the light-emitting device may be enhanced or improved.

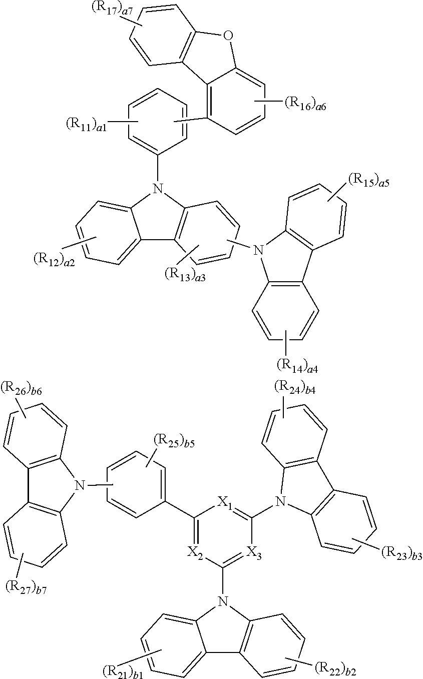

### CROSS-REFERENCE TO RELATED APPLICATION

This application claims priority to and the benefit of Korean Patent Application No. 10-2023-0003028, filed on Jan. 9, 2023, in the Korean Intellectual Property Office, the content of which is incorporated by reference herein in its entirety.

### BACKGROUND

### 1. Field

One or more aspects of embodiments of the present disclosure relate to a composition, a light-emitting device, and an electronic apparatus including the same.

### 2. Description of the Related Art

Self-emissive devices (for example, organic light-emitting devices) are a class of light-emitting devices have relatively wide viewing angles, high contrast ratios, short response times, and excellent or suitable characteristics in terms of luminance, driving voltage, and response speed.

In a light-emitting device, a first electrode is provided on a substrate, and a hole transport region, an emission layer, an electron transport region, and a second electrode are sequentially provided on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. These excitons may transition (i.e., relax) from an excited state to a ground state to thereby generate light.

Implementation of the light-emitting device in a display device requires (or there is a desire) that the light-emitting device (e.g., self-emissive device) possess reduced driving voltage, improved luminescence efficiency, and/or a long lifespan. Therefore, the need exists for development of a material for a light-emitting device which is capable of stably (or suitably) implementing these properties. For example, to implement a light-emitting device having high luminescence efficiency, materials for a hole transport region having excellent or suitable energy levels and phase transition temperature properties are continuously being developed and/or desired.

### SUMMARY

One or more aspects of embodiments of the present disclosure are directed toward a composition capable of providing improved luminescence efficiency and lifespan characteristics, a light-emitting device, and an electronic apparatus each including the composition. One or more aspects of embodiments of the present disclosure are directed toward a composition including a first compound and a second compound, a light-emitting device with improved luminescence efficiency, and an electronic apparatus including the same.

Additional aspects will be set forth in part in the description which follows and, in part, will be apparent from the description, or may be learned by practice of the presented embodiments of the disclosure.

According to one or more embodiments, a composition includes a first compound represented by Formula 1, and a second compound represented by Formula 2:

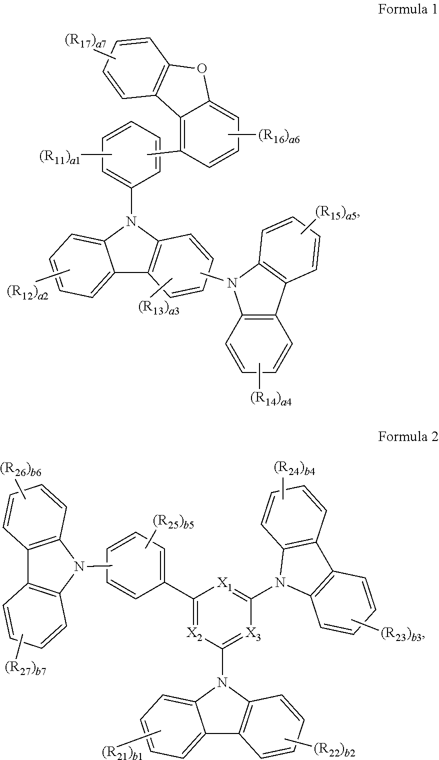

wherein, in Formulae 1 and 2,

R₁₁ to R₁₇ and R₂₁ to R₂₇ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),

a1 to a7 and b1 to b7 may each independently be an integer from 1 to 4,

X₁ may be N or C(Y₁),

X₂ may be N or C(Y₂),

X₃ may be N or C(Y₃),

at least one of selected from among X₁ to X₃ may be N,

Y₁ to Y₃ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),

R₁₀ₐ may be

deuterium, —F, —Cl, —Br, —I, a hydroxyl group, a cyano group, or a nitro group,

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or any combination thereof,

a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or any combination thereof, or

—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂), and

Q₁ to Q₃, Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group or a C₁-C₆ heterocyclic group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or any combination thereof.

According to one or more embodiments, a light-emitting device includes a first electrode, a second electrode facing the first electrode, an interlayer arranged or located between the first electrode and the second electrode and including an emission layer. In one or more embodiments, the emission layer includes the first compound and the second compound.

According to one or more embodiments, a method of manufacturing the light-emitting device includes forming a composition-containing layer by filling the composition in a deposition source in a vacuum chamber and performing a deposition process of heating the composition.

According to one or more embodiments, an electronic apparatus includes the light-emitting device.

According to one or more embodiments, an electronic equipment includes the light-emitting device.

### BRIEF DESCRIPTION OF THE DRAWINGS

The accompanying drawings are included to provide a further understanding of the above and other aspects, features, and advantages of certain embodiments of the present disclosure and are incorporated in and constitute a part of this specification. The drawings illustrate example embodiments and, together with the following description taken in conjunction with the accompanying drawings, serve to make the principles of the present disclosure more apparent. In the drawings:

FIG. 1 is a schematic cross-sectional view of a light-emitting device according to one or more embodiments of the present disclosure;

FIG. 2 is a cross-sectional view of an electronic apparatus according to one or more embodiments of the present disclosure;

FIG. 3 is a cross-sectional view of an electronic apparatus according to one or more embodiments of the present disclosure;

FIG. 4 is a schematic perspective view of electronic equipment including a light-emitting device according to one or more embodiments of the present disclosure;

FIG. 5 is a schematic perspective view of the exterior of a vehicle; and

FIG. 6A is a schematic view of the interior of a vehicle that includes electronic equipment according to one or more embodiments of the present disclosure;

FIG. 6B is a schematic view of the interior of a vehicle that includes electronic equipment according to one or more embodiments of the present disclosure; and

FIG. 6C is a schematic view of the interior of a vehicle that includes electronic equipment according to one or more embodiments of the present disclosure.

### DETAILED DESCRIPTION

Reference will now be made in more detail to embodiments of the present disclosure, examples of which are illustrated in the accompanying drawings, wherein like reference numerals refer to like elements throughout, and duplicative descriptions thereof may not be provided. In this regard, the present embodiments may have different forms and should not be construed as being limited to the descriptions set forth herein. Accordingly, the embodiments are merely described, by referring to the drawings, to explain aspects of the present description.

As utilized herein, the term “and/or” includes any, and all, combinations of one or more of the associated listed items. Expressions such as “at least one of,” “one of,” and “selected from,” when preceding a list of elements, modify the entire list of elements and do not modify the individual elements of the list. For example, throughout the disclosure, the expression “at least one of a, b or c” indicates only a, only b, only c, both (e.g., simultaneously) a and b, both (e.g., simultaneously) a and c, both (e.g., simultaneously) b and c, all of a, b, and c, or variations thereof.

The terminology used herein is for the purpose of describing embodiments and is not intended to limit the embodiments described herein. Unless otherwise defined, all chemical names, technical and scientific terms, and terms defined in common dictionaries should be interpreted as having meanings consistent with the context of the related art, and should not be interpreted in an ideal or overly formal sense. It will be understood that, although the terms first, second, etc. may be used herein to describe various elements, these elements should not be limited by these terms. These terms are only used to distinguish one element from another element. Thus, a first element could be termed a second element without departing from the teachings of the present disclosure. Similarly, a second element could be termed a first element.

As used herein, singular forms such as “a,” “an,” and “the” are intended to include the plural forms as well, unless the context clearly indicates otherwise.

It will be further understood that the terms “comprise,” “comprises,” “comprising,” “has,” “have,” “having,” “include,” “includes,” and/or “including,” when used in this specification, specify the presence of stated features, steps, operations, elements, and/or components, but do not preclude the presence or addition of one or more other features, steps, operations, elements, components, and/or groups thereof.

As used herein, the terms “use,” “using,” and “used” may be considered synonymous with the terms “utilize,” “utilizing,” and “utilized,” respectively.

As used herein, the term “and/or” includes any, and all, combination(s) of one or more of the associated listed items.

The term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

It will be understood that when an element is referred to as being “on,” “connected to,” or “on” another element, it may be directly on, connected, or coupled to the other element or one or more intervening elements may also be present. When an element is referred to as being “directly on,” “directly connected to,” or “directly coupled to” another element, there are no intervening elements present.

Spatially relative terms, such as “beneath,” “below,” “lower,” “above,” “upper,” “bottom,” “top,” and the like, may be used herein for ease of description to describe one element or feature's relationship to another element(s) or feature(s) as illustrated in the drawings. It will be understood that the spatially relative terms are intended to encompass different orientations of the device in use or operation in addition to the orientation depicted in the drawings. For example, if the device in the drawings is turned over, elements described as “below” or “beneath” other elements or features would then be oriented “above” or “over” the other elements or features. Thus, the term “below” may encompass both an orientation of above and below. The device may be otherwise oriented (rotated 90 degrees or at other orientations), and the spatially relative descriptors used herein should be interpreted accordingly.

Unless otherwise defined, all terms (including technical and scientific terms) used herein have the same meaning as commonly understood by those of ordinary skill in the art to which the present disclosure pertains. It is also to be understood that terms defined in commonly used dictionaries should be interpreted as having meanings consistent with meanings in the context of the related art, unless expressly defined herein, and should not be interpreted in an ideal or overly formal sense.

In this context, “consisting essentially of” means that any additional components will not materially affect the chemical, physical, optical or electrical properties of the semiconductor film.

Further, in this specification, the phrase “on a plane,” or “plan view,” means viewing a target portion from the top, and the phrase “on a cross-section” means viewing a cross-section formed by vertically cutting a target portion from the side.

According to an aspect of the disclosure, a composition includes:

a first compound represented by Formula 1; and

a second compound represented by Formula 2:

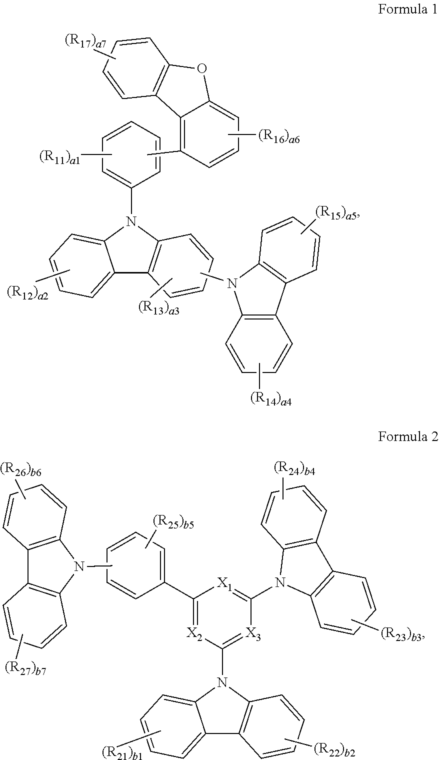

wherein Formulae 1 and 2 are respectively the same as described in the specification.

Methods of synthesizing the first compound and the second compound may be easily understood to those of ordinary skill in the art by referring to Synthesis Examples and/or Examples described herein.

In one or more embodiments, the composition may be a composition for forming an organic light-emitting device.

In one or more embodiments, the composition may be included in a layer including: 1) the first compound and the second compound; and 2) a transition metal-containing compound, a delayed fluorescence compound, or any combination thereof. The layer including the composition may include a mixture including: 1) the first compound and the second compound; and 2) a transition metal-containing compound, a delayed fluorescence compound, or any combination thereof. Therefore, the layer including the composition is clearly differentiated from, for example, a double layer including: 1) a first layer including the first compound and the second compound; and 2) a second layer including a transition metal-containing compound, a delayed fluorescence compound, or any combination thereof.

In one or more embodiments, the composition may be a composition prepared to form a layer including: 1) the first compound and the second compound; and 2) a transition metal-containing compound, a delayed fluorescence compound, or any combination thereof by utilizing one or more suitable methods such as a deposition method and a wet process. In one or more embodiments, the composition may be a pre-mixed mixture prepared for utilization in a deposition method (for example, a vacuum deposition method). The pre-mixed mixture may be charged, for example, into a deposition source within a vacuum chamber, and two or more compounds included in the pre-mixed mixture may be co-deposited.

In one or more embodiments, the composition may further include a transition metal-containing compound, a delayed fluorescence compound, or any combination thereof.

In one or more embodiments, a difference between a phase transition temperature of the first compound under a pressure of about 5.0×10⁻⁵ torr to about 1.0×10⁻³ torr and a phase transition temperature of the second compound under a pressure of 5.0×10⁻⁵ torr to about 1.0×10⁻³ torr may be in a range of about 20° C. or less, about 0° C. to about 20° C., about 1° C. to about 20° C., about 2° C. to about 20° C., about 3° C. to about 20° C., about 4° C. to about 20° C., about 5° C. to about 20° C., about 0° C. to about 18° C., about 1° C. to about 18° C., about 2° C. to about 18° C., about 3° C. to about 18° C., about 4° C. to about 18° C., about 5° C. to about 18° C., about 0° C. to about 15° C., about 1° C. to about 15° C., about 2° C. to about 15° C., about 3° C. to about 15° C., about 4° C. to about 15° C., about 5° C. to about 15° C., about 0° C. to about 12° C., about 1° C. to about 12° C., about 2° C. to about 12° C., about 3° C. to about 12° C., about 4° C. to about 12° C., about 5° C. to about 12° C., about 0° C. to about 10° C., about 1° C. to about 10° C., about 2° C. to about 10° C., about 3° C. to about 10° C., about 4° C. to about 10° C., or about 5° C. to about 10° C.

In one or more embodiments, a difference between a phase transition temperature of the first compound and a phase transition temperature of the second compound may be in a range of about 20° C. or less, about 0° C. to about 20° C., about 1° C. to about 20° C., about 2° C. to about 20° C., about 3° C. to about 20° C., about 4° C. to about 20° C., about 5° C. to about 20° C., about 0° C. to about 18° C., about 1° C. to about 18° C., about 2° C. to about 18° C., about 3° C. to about 18° C., about 4° C. to about 18° C., about 5° C. to about 18° C., about 0° C. to about 15° C., about 1° C. to about 15° C., about 2° C. to about 15° C., about 3° C. to about 15° C., about 4° C. to about 15° C., about 5° C. to about 15° C., about 0° C. to about 12° C., about 1° C. to about 12° C., about 2° C. to about 12° C., about 3° C. to about 12° C., about 4° C. to about 12° C., about 5° C. to about 12° C., about 0° C. to about 10° C., about 1° C. to about 10° C., about 2° C. to about 10° C., about 3° C. to about 10° C., about 4° C. to about 10° C., or about 5° C. to about 10° C. The phase transition temperature was evaluated under the same pressure, and the pressure may be about 5.0×10⁻⁵ torr to about 1.0×10⁻³ torr.

In one or more embodiments, the phase transition temperature of the first compound may be about 285° C. to about 305° C.

In one or more embodiments, the phase transition temperature of the second compound may be about 285° C. to about 305° C.

The first compound and the second compound satisfy a phase transition temperature relationship as described, and thus phase transitions of the first compound and the second compound in the composition (for example, a pre-mixed mixture) including the first compound and the second compound may be made at substantially the same temperature within the range of the pressure. Therefore, when a deposition process is performed after the composition including the first compound and the second compound is charged to a deposition source, the first compound and the second compound in the composition may be vaporized at substantially the same temperature, and thus the first compound and the second compound may be co-deposited, (e.g., effectively co-deposited), and one or more suitable electrical characteristics and durability of a layer prepared as a result of the co-deposition may be enhanced or improved.

In one or more embodiments, an amount of the first compound may be about 1 part by weight to 99 parts by weight based on 100 parts by weight of the composition, and an amount of the second compound may be 1 part by weight to 99 parts by weight based on 100 parts by weight of the composition.

According to another aspect, a light-emitting device includes: a first electrode; a second electrode facing the first electrode; an interlayer provided or arranged between the first electrode and the second electrode and including an emission layer; the first compound; and the second compound.

As the light-emitting device includes the first compound and the second compound, the light-emitting device may have enhanced or improved luminescence efficiency and lifespan characteristics, and may also have enhanced or improved electrical characteristics and durability.

In one or more embodiments, the first compound and the second compound may be included in an interlayer of the light-emitting device.

In one or more embodiments, the first compound and the second compound may be included in an emission layer of the light-emitting device.

In one or more embodiments, the emission layer may include a transition metal-containing compound, a delayed fluorescence compound, or any combination thereof. The first compound, the second compound, the transition metal-containing compound, and the delayed fluorescence compound of the emission layer may be different from each other.

In one or more embodiments, the emission layer may include a luminescent material.

In one or more embodiments, the luminescent material may include a transition metal-containing compound, a delayed fluorescence compound, or any combination thereof. In one or more embodiments, the transition metal-containing compound and the delayed fluorescence compound of the luminescent material may be different from each other.

In one or more embodiments, the transition metal-containing compound and the delayed fluorescence compound of the composition and the light-emitting device are respectively the same as described in the specification.

In one or more embodiments, the first compound, the second compound, the transition metal-containing compound, the delayed fluorescence compound, or any combination thereof may include at least one deuterium.

For example, the first compound may include at least one deuterium.

In another example, the second compound may include at least one deuterium.

In another example, the transition metal-containing compound and the delayed fluorescence compound may each include at least one deuterium.

In one or more embodiments, each of the composition and the light-emitting device may further include a transition metal-containing compound and a delayed fluorescence compound, in addition to the first compound and the second compound, and at least one of selected from among the first compound, the second compound, the transition metal-containing compound, and the delayed fluorescence compound may include at least one deuterium.

In one or more embodiments, the composition and the light-emitting device (for example, the emission layer in the light-emitting device) may each further include a transition metal-containing compound, in addition to the first compound and the second compound. At least one of selected from among the first compound, the second compound, and the transition metal-containing compound may include at least one deuterium. For example, the composition and the light-emitting device (for example, the emission layer in the light-emitting device) may each further include a delayed fluorescence compound, in addition to the first compound, the second compound, and the transition metal-containing compound.

In one or more embodiments, the composition and the light-emitting device (for example, the emission layer in the light-emitting device) may each further include a delayed fluorescence compound, in addition to the first compound and the second compound. At least one of selected from among the first compound, the second compound, and the delayed fluorescence compound may include at least one deuterium. The delayed fluorescence compound may serve to improve color purity, luminescence efficiency, and lifespan characteristics of the light-emitting device. For example, the composition and the light-emitting device (for example, the emission layer in the light-emitting device) may each further include a transition metal-containing compound, in addition to the first compound, the second compound, and the delayed fluorescence compound.

In one or more embodiments, the first compound and the second compound may form an exciplex. At least one of selected from among the first compound, the second compound, and the transition metal-containing compound may include at least one deuterium.

In one or more embodiments, a highest occupied molecular orbital (HOMO) energy level of the first compound may be −5.5 electron-volt (eV) or more. For example, the HOMO energy level of the first compound may be about −5.5 eV to about −4.9 eV, about −5.5 eV to about −5.0 eV, about −5.5 eV to about −5.1 eV, about −5.5 eV to about −5.2 eV, or about −5.5 eV to about −5.3 eV.

In one or more embodiments, a lowest unoccupied molecular orbital (LUMO) energy level of the second compound may be −2.6 eV or less. For example, the LUMO energy level of the second compound may be about −2.6 eV to about −2.0 eV, about −2.5 eV to about −2.0 eV, about −2.4 eV to about −2.0 eV, about −2.5 eV to about −2.0 eV, or about −2.4 eV to about −2.0 eV.

For example, the HOMO energy level and the LUMO energy level may be evaluated through a cyclic voltammetry analysis of the first compound and the second compound.

In one or more embodiments, a triplet (T1) energy level of each of the first compound and the second compound may be 2.8 eV or more. For example, the triplet (T1) energy level of each of the first compound and the second compound may be about 2.7 eV to about 3.4 eV, about 2.7 eV to about 3.3 eV, about 2.7 eV to about 3.2 eV, about 2.7 eV to about 3.1 eV, or about 2.7 eV to about 3.0 eV.

For example, the HOMO energy level, the LUMO energy level, and the triplet (T1) energy level may be evaluated through quantum chemical calculation of the first compound and the second compound.

As the first compound and the second compound satisfy the foregoing HOMO energy level, the LUMO energy level, or the triplet (T1) energy level, the first compound and the second compound may have high luminescence efficiency and long lifespan.

In one or more embodiments, a maximum emission wavelength (or an emission peak wavelength) of a photoluminescence spectrum in a film of the transition metal-containing compound may be in a range of about 400 nanometer (nm) to about 500 nm, about 410 nm to about 490 nm, about 420 nm to about 480 nm, about 430 nm to about 475 nm, about 440 nm to about 475 nm, about 450 nm to about 475 nm, about 430 nm to about 470 nm, about 440 nm to about 470 nm, about 450 nm to about 470 nm, about 430 nm to about 465 nm, about 440 nm to about 465 nm, about 450 nm to about 465 nm, about 430 nm to about 460 nm, about 440 nm to about 460 nm, or about 450 nm to about 460 nm.

In one or more embodiments, i) the first compound and the second compound; and ii) the transition metal-containing compound, or the delayed fluorescence compound may be included in the emission layer of the light-emitting device, and the emission layer may be to emit blue light or blue-green light.

In one or more embodiments, a maximum emission wavelength of light emitted from the emission layer may be about 400 nm to about 500 nm, about 410 nm to about 490 nm, about 420 nm to about 480 nm, about 430 nm to about 475 nm, about 440 nm to about 475 nm, about 450 nm to about 475 nm, about 430 nm to about 470 nm, about 440 nm to about 470 nm, about 450 nm to about 470 nm, about 430 nm to about 465 nm, about 440 nm to about 465 nm, about 450 nm to about 465 nm, about 430 nm to about 460 nm, about 440 nm to about 460 nm, or about 450 nm to about 460 nm.

In one or more embodiments, the blue light may be deep blue light.

In one or more embodiments, a CIEx coordinate (for example, a bottom emission CIEx coordinate) of the blue light may be in a range of about 0.125 to about 0.140 or about 0.130 to about 0.140.

In one or more embodiments, a CIEy coordinate (for example, a bottom emission CIEy coordinate) of the blue light may be in a range of about 0.120 to about 0.200.

In one or more embodiments, the transition metal-containing compound may include platinum (Pt).

In one or more embodiments, the transition metal-containing compound may include platinum and a tetradentate ligand. In one or more embodiments, the tetradentate ligand may be bonded to the platinum. In one or more embodiments, the platinum and a carbon atom (e.g., one of the carbon atoms) of the tetradentate ligand may be bonded to each other via a coordinate bond.

In one or more embodiments, the transition metal-containing compound may be a carbene-containing compound.

In one or more embodiments, the transition metal-containing compound may be a compound represented by Formula 3. Formula 3 is the same as described in the specification.

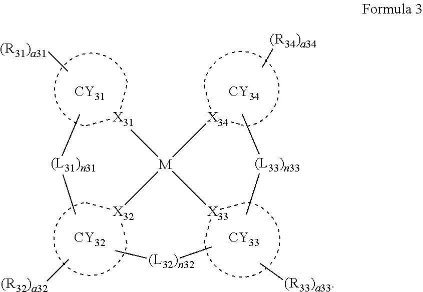

In one or more embodiments, a difference between a triplet energy level (in eV units) of the delayed fluorescence compound and a singlet energy level (in eV units) of the delayed fluorescence compound may be about 0 eV or higher and about 0.5 eV or lower (or, about 0 eV or higher and about 0.3 eV or lower).

In one or more embodiments, the delayed fluorescence compound may be a compound including at least one cyclic group. In one or more embodiments, the at least one cyclic group includes each of boron (B) and nitrogen (N), as a ring-forming atom. In one or more embodiments, the at least one cyclic group includes boron (B) and nitrogen (N).

In some embodiments, the delayed fluorescence compound may be a C₈-C₆₀ polycyclic group-containing compound including at least two condensed cyclic groups that share a boron atom (B).

In one or more embodiments, the delayed fluorescence compound may include a condensed ring. In one or more embodiments, the delayed fluorescence compound may include a third ring and a fourth ring. In one or more embodiments, the delayed fluorescence compound includes a condensed ring in which at least one third ring may be condensed with at least one fourth ring,

In one or more embodiments, the third ring may be a cyclopentane group, a cyclohexane group, a cycloheptane group, a cyclooctane group, a cyclopentene group, a cyclohexene group, a cycloheptene group, a cyclooctene group, an adamantane group, a norbornene group, a norbornane group, a bicyclo[1.1.1]pentane group, a bicyclo[2.1.1]hexane group, a bicyclo[2.2.2]octane group, a benzene group, a pyridine group, a pyrimidine group, a pyridazine group, a pyrazine group, or a triazine group.

In one or more embodiments, the fourth ring may be a 1,2-azaborinine group, a 1,3-azaborinine group, a 1,4-azaborinine group, a 1,2-dihydro-1,2-azaborinine group, a 1,4-oxaborinine group, a 1,4-thiaborinine group, or a 1,4-dihydroborinine group.

In some embodiments, the delayed fluorescence compound may be a compound represented by Formula 502, a compound represented by Formula 503, or any combination thereof:

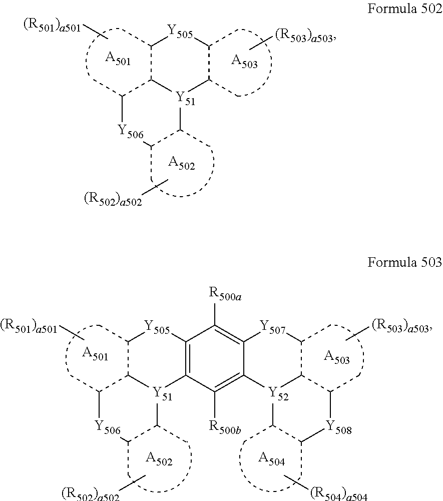

wherein, in Formulae 502 and 503,

ring A₅₀₁ to ring A₅₀₄ may each independently be a C₃-C₆₀ carbocyclic group or a C₁-C₆₀ heterocyclic group,

Y₅₅₀ may be O, S, N(R₅₀₅), B(R₅₀₅), C(R₅₀₅ₐ)(R_{505b}), or Si(R₅₀₅ₐ)(R_{505b}),

Y₅₀₆ may be O, S, N(R₅₀₆), B(R₅₀₆), C(R₅₀₆ₐ)(R_{506b}), or Si(R₅₀₆ₐ)(R_{506b}),

Y₅₀₇ may be O, S, N(R₅₀₇), B(R₅₀₇), C(R₅₀₇ₐ)(R_{507b}), or Si(R₅₀₇ₐ)(R_{507b}),

Y₅₀₈ may be O, S, N(R₅₀₈), B(R₅₀₈), C(R₅₀₅ₐ)(R_{505b}), or Si(R₅₀₅ₐ)(R_{505b}),

Y₅₁ and Y₅₂ may each independently be B, P(═O), or S(═O),

R₅₀₀ₐ, R_{500b}, R₅₀₁ to R₅₀₈, R₅₀₅ₐ, R_{505b}, R₅₀₆ₐ, R_{506b}, R₅₀₇ₐ, R_{507b}, R₅₀₅ₐ, and R_{505b} are respectively the same as described elsewhere in the specification, and

a501 to a504 may each independently be an integer from 0 to 20.

In one or more embodiments, the light-emitting device may satisfy at least one of selected from among Conditions 1 to 4:

### Condition 1

LUMO energy level (eV) of the first compound>LUMO energy level (eV) of the transition metal-containing compound;

### Condition 2

LUMO energy level (eV) of the transition metal-containing compound >LUMO energy level (eV) of the second compound;

### Condition 3

HOMO energy level (eV) of the transition metal-containing compound >HOMO energy level (eV) of the first compound; and

### Condition 4

HOMO energy level (eV) of the first compound >HOMO energy level (eV) of the second compound.

Each of the HOMO energy level and LUMO energy level of each of the first compound, the second compound, and the transition metal-containing compound may be a negative value, and may be measured according to a suitable method.

In one or more embodiments, an absolute value of a difference between a LUMO energy level of the transition metal-containing compound and a LUMO energy level of the second compound may be about 0.1 eV or higher and about 1.0 eV or lower. In one or more embodiments, an absolute value of a difference between a LUMO energy level of the transition metal-containing compound and a LUMO energy level of the first compound may be about 0.1 eV or higher and about 1.0 eV or lower. In one or more embodiments, an absolute value of a difference between a HOMO energy level of the transition metal-containing compound and a HOMO energy level of the second compound may be about 1.25 eV or lower (for example, about 1.25 eV or lower and about 0.2 eV or higher). In one or more embodiments, an absolute value of a difference between a HOMO energy level of the transition metal-containing compound and a HOMO energy level of the first compound may be about 1.25 eV or lower (for example, about 1.25 eV or lower and about 0.2 eV or higher).

When the relationships between LUMO energy level and HOMO energy level satisfy the conditions as described, the balance between holes and electrons injected into the emission layer can be made, e.g., the amount of holes and the amount of electrons injected into the emission layer may be optimized or suitable for the purposes described herein.

In one or more embodiments, the light-emitting device may have a structure of a first embodiment or a second embodiment.

### First Embodiment

According to the first embodiment, the first compound and the second compound may be included in the emission layer of the interlayer of the light-emitting device, and the emission layer may further include a transition metal-containing compound. According to the first embodiment, the emission layer may be to emit phosphorescence or fluorescence emitted from the transition metal-containing compound. For example, according to the first embodiment, the first compound and the second compound may be a host, and the transition metal-containing compound may be a dopant or an emitter. In one or more embodiments, the transition metal-containing compound may be a phosphorescent dopant or a phosphorescent emitter.

The phosphorescence or fluorescence emitted from the transition metal-containing compound may be blue light.

The emission layer may further include an auxiliary dopant. The auxiliary dopant may serve to improve luminescence efficiency from the first compound by effectively transferring energy to the transition metal-containing compound as a dopant or an emitter.

The auxiliary dopant may be different from each of the transition metal-containing compound, the first compound, and the second compound.

In one or more embodiments, the auxiliary dopant may be a delayed fluorescence-emitting compound.

In some embodiments, the auxiliary dopant may be a compound including at least one cyclic group including boron (B) and nitrogen (N) as ring-forming atoms.

### Second Embodiment

According to the second embodiment, the first compound and the second compound may be included in the emission layer of the interlayer of the light-emitting device, the emission layer may further include a transition metal-containing compound and a dopant, and the first compound, the second compound, the transition metal-containing compound, and the dopant may be different from each other. According to the second embodiment, the emission layer may be to emit phosphorescence or fluorescence (e.g., delayed fluorescence) emitted from the dopant. For example, according to the second embodiment, the first compound and the second compound may be a host, the transition metal-containing compound may not be a dopant but serve as an auxiliary dopant transmitting energy to the dopant (or emitter).

In another example, the first compound and the second compound in the second embodiment may be a host, and the transition metal-containing compound may serve as an emitter as well as an auxiliary dopant transmitting energy to the dopant (or emitter).

For example, phosphorescence or fluorescence emitted from the dopant (or the emitter) in the second embodiment may be blue phosphorescence or blue fluorescence (e.g., blue delayed fluorescence).

In the second embodiment, the dopant (or emitter) may be a phosphorescent dopant material (for example, a material of the transition metal-containing compound in the specification) or a material of any fluorescent dopant (for example, a compound represented by Formula 501, a compound represented by Formula 502, a compound represented by Formula 503, or any combination thereof in the specification).

In the first and second embodiments, the blue light may be blue light having a maximum emission wavelength of about 400 nm to about 500 nm, about 410 nm to about 490 nm, about 420 nm to about 480 nm, about 430 nm to about 475 nm, about 440 nm to about 475 nm, about 450 nm to about 475 nm, about 430 nm to about 470 nm, about 440 nm to about 470 nm, about 450 nm to about 470 nm, about 430 nm to about 465 nm, about 440 nm to about 465 nm, about 450 nm to about 465 nm, about 430 nm to about 460 nm, about 440 nm to about 460 nm, or about 450 nm to about 460 nm.

In one or more embodiments, the auxiliary dopant in the first embodiment may include the delayed fluorescence compound represented by Formula 502 or Formula 503.

The host in the first embodiment and the second embodiment may further include any host material (e.g., a compound represented by Formula 301, a compound represented by 301-1, a compound represented by Formula 301-2, or any combination thereof).

In one or more embodiments, the light-emitting device may further include a capping layer located outside the first electrode and/or outside the second electrode.

In another embodiment, the light-emitting device may further include at least one of a first capping layer located on or arranged outside the first electrode and a second capping layer located on or arranged outside the second electrode, and at least one of selected from among the first capping layer and the second capping layer may include the first compound represented by Formula 1 and the second compound represented by Formula 2. The first capping layer and/or the second capping layer may each be the same as described herein.

In one or more embodiments, the light-emitting device may include: a first capping layer located or arranged outside the first electrode and including the first compound represented by Formula 1 and the second compound represented by Formula 2; a second capping layer located or arranged outside the second electrode and including the first compound represented by Formula 1 and the second compound represented by Formula 2; or the first capping layer and the second capping layer.

The wording “(interlayer and/or capping layer) includes a first compound represented by Formula 1 and a second compound represented by Formula 2” as utilized herein may be understood as “(interlayer and/or capping layer) may include one kind of first compound represented by Formula 1 or two different kinds of first compounds, each represented by Formula 1; and one kind of second compound represented by Formula 2 or two different kinds of second compound, each represented by Formula 2.”

For example, the interlayer and/or the capping layer may only include Compound H3 as the first compound and Compound E1 as the second compound. In this regard, Compounds H3 and E1 may be present in the emission layer of the light-emitting device. Or the interlayer may include Compounds H3 and H8 as the first compound and Compounds E1 and E4 as the second compound. In one or more embodiments, Compounds H3 and H8 and Compounds E1 and E4 may be present in substantially the same layer (for example, both (e.g., simultaneously) Compounds H3 and H8 may be present in the emission layer, and both (e.g., simultaneously) Compounds E1 and E4 may be present in the emission layer). In one or more embodiments, Compounds H3 and H8 and Compounds E1 and E4 may be present in different layers (for example, Compound H3 may be present in the emission layer whereas Compound H8 may be present in the hole transport region, and Compound E1 may be present in the emission layer whereas Compound E4 may be present in the electron transport region.)

The term “interlayer” as utilized herein refers to a single layer and/or all layers between a first electrode and a second electrode of a light-emitting device.

Another aspect of the disclosure provides a method of manufacturing a light-emitting device, the method including forming a composition-containing layer by filling a deposition source in a vacuum chamber with the composition and performing a deposition process of heating the composition.

In one or more embodiments, the interlayer may be the composition-containing layer.

In one or more embodiments, the interlayer may have a multi-layered structure and include the composition-containing layer.

In one or more embodiments, the composition-containing layer may be the emission layer.

Another aspect of the disclosure provides an electronic apparatus including the light-emitting device. The electronic apparatus may further include a thin-film transistor. For example, the electronic apparatus may further include a thin-film transistor including a source electrode and a drain electrode, wherein the first electrode of the light-emitting device may be electrically connected to the source electrode or the drain electrode. In one or more embodiments, the electronic apparatus may further include a color filter, a color conversion layer, a touch screen layer, a polarizing layer, or any combination thereof. More details of the electronic apparatus may be referred to the descriptions provided herein.

Another aspect of the disclosure provides electronic equipment including the light-emitting device.

For example, the electronic equipment may be one selected from among a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, and/or a signboard.

### Description of Formula 1

In one or more embodiments, provided is a first compound represented by Formula 1:

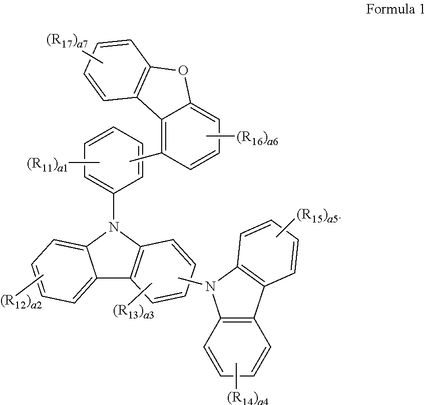

In Formula 1, R₁₁ to R₁₇ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂).

In Formula 1, a1 to a7 may each independently be an integer from 1 to 4,

R₁₀ₐ may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂), and

Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or any combination thereof.

In one or more embodiments, R₁₁ to R₁₇ may each independently be:

hydrogen, deuterium, —F, —Cl, —Br, —I, a hydroxyl group, a cyano group, a nitro group, a C₁-C₂₀ alkyl group, or a C₁-C₂₀ alkoxy group;

a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₁ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzothiazolyl group, a benzoisoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, —O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or any combination thereof; or

—C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

In one or more embodiments, R₁₁ to R₁₇ may each independently be:

hydrogen or deuterium;

a C₁-C₂₀ alkyl group unsubstituted or substituted with at least one deuterium; or

a phenyl group unsubstituted or substituted with deuterium, a C₁-C₂₀ alkyl group, a phenyl group, a biphenyl group, or any combination thereof.

In one or more embodiments, R₁₁ to R₁₇ may each independently be hydrogen, deuterium, a phenyl group, or a deuterated phenyl group.

In one or more embodiments, R₁₁ to R₁₇ may each independently be hydrogen or deuterium.

In one or more embodiments, the first compound may be a group represented by any one selected from among Formulae 1-1 to 1-5:

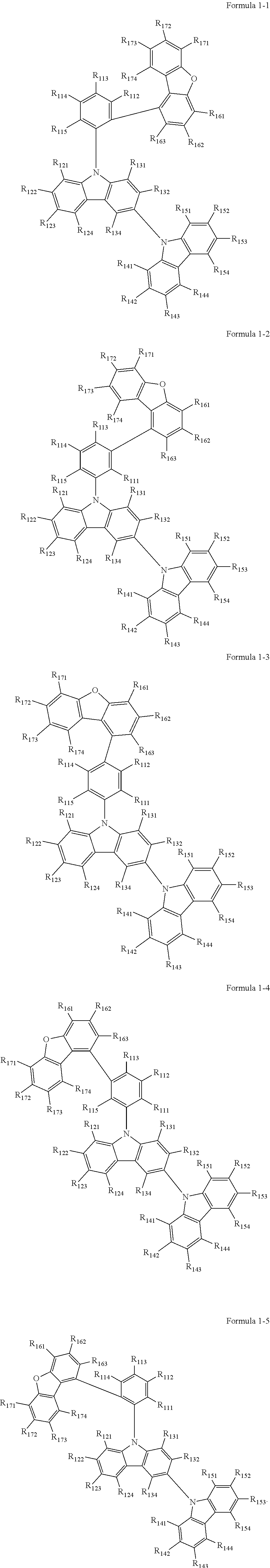

In Formulae 1-1 to 1-5,

R₁₁₁ to R₁₁₃ are each the same as described herein in connection with R₁₁,

R₁₂₁ to R₁₂₄ are each the same as described herein in connection with R₁₂,

R₁₃₁, R₁₃₂, and R₁₃₄ are each the same as described herein in connection with R₁₃,

R₁₄₁ to R₁₄₄ are each the same as described herein in connection with R₁₄,

R₁₅₁ to R₁₅₄ are each the same as described herein in connection with R₁₅,

R₁₆₁ to R₁₆₃ are each the same as described herein in connection with R₁₆, and

R₁₇₁ to R₁₇₄ are each the same as described herein in connection with R₁₇.

In Formulae 1-1 to 1-5, R₁₁₁ to R₁₁₅, R₁₂₁ to R₁₂₄, R₁₃₁, R₁₃₂, R₁₃₄, R₁₄₁ to R₁₄₄, R₁₅₁ to R₁₅₄, R₁₆₁ to R₁₆₃, and R₁₇₁ to R₁₇₄ may each independently be hydrogen or deuterium.

For example, at least four of R₁₁₁ to R₁₁₅, R₁₂₁ to R₁₂₄, R₁₃₁, R₁₃₂, R₁₃₄, R₁₄₁ to R₁₄₄, R₁₅₁ to R₁₅₄, R₁₆₁ to R₁₆₃, and R₁₇₁ to R₁₇₄ may be deuterium.

### Description of Formula 2

In one or more embodiments, provided is a second compound represented by Formula 2:

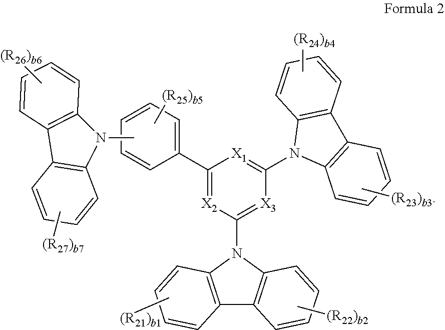

In Formula 2, R₂₁ to R₂₇ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂).

In Formula 2, b1 to b7 may each independently be an integer from 1 to 4,

X₁ may be N or C(Y₁),

X₂ may be N or C(Y₂),

X₃ may be N or C(Y₃),

at least one selected from among X₁ to X₃ may be N,

Y₁ to Y₃ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),

R₁₀ₐ may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂), and

Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or any combination thereof.

In one or more embodiments, R₂₁ to R₂₇ may each independently be:

hydrogen, deuterium, —F, —Cl, —Br, —I, a hydroxyl group, a cyano group, a nitro group, a C₁-C₂₀ alkyl group, or a C₁-C₂₀ alkoxy group;

a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzothiazolyl group, a benzoisoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, —O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or any combination thereof; or

—C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

In one or more embodiments, R₂₁ to R₂₇ may each independently be:

hydrogen or deuterium;

a C₁-C₂₀ alkyl group unsubstituted or substituted with at least one deuterium; or

a phenyl group unsubstituted or substituted with deuterium, a C₁-C₂₀ alkyl group, a phenyl group, a biphenyl group, or any combination thereof.

In one or more embodiments, R₂₁ to R₂₇ may each independently be hydrogen, deuterium, a phenyl group, or a deuterated phenyl group.

In one or more embodiments, R₂₁ to R₂₇ may each independently be hydrogen or deuterium.

In one or more embodiments, at least two selected from among X₁ to X₃ may be N.

In one or more embodiments, the second compound may be a group represented by any one selected from among Formulae 2-1 to 2-5:

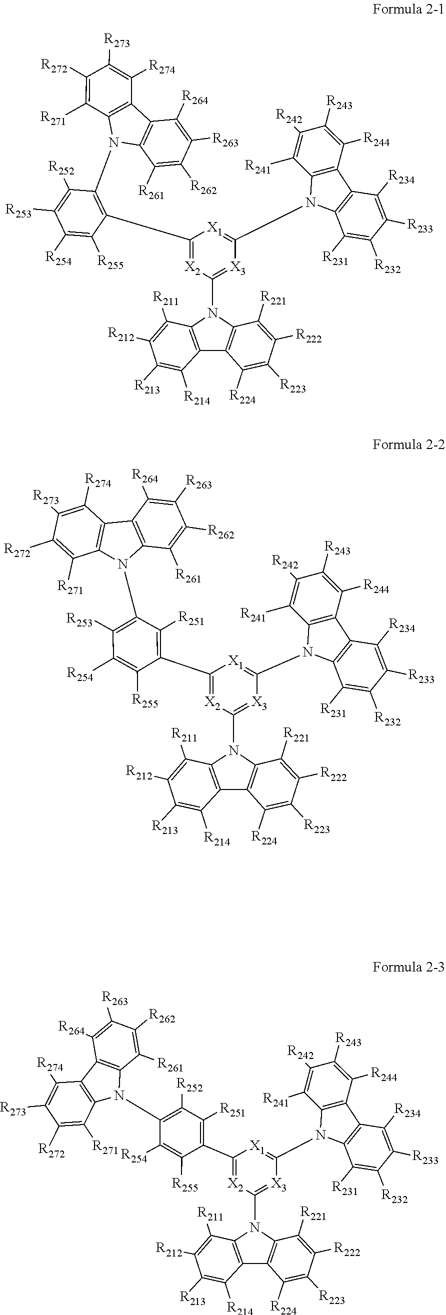

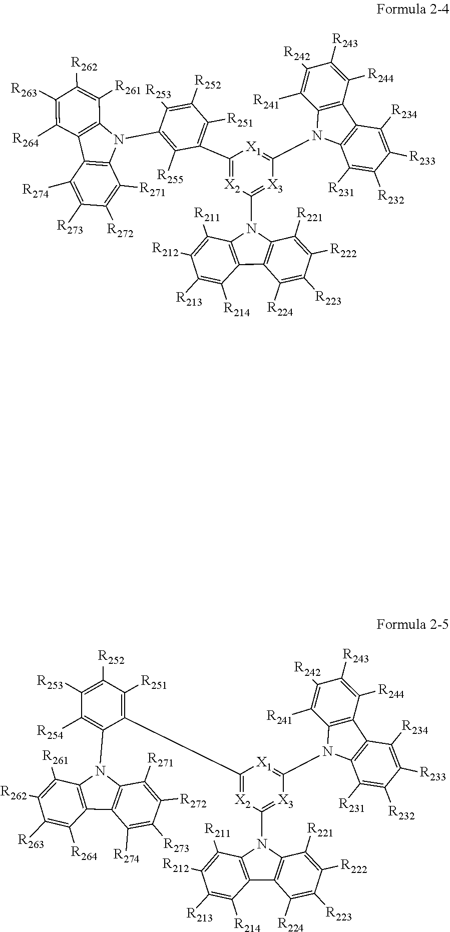

wherein, in Formulae 2-1 to 2-5,

R₂₁₁ to R₂₁₄ are each the same as described herein in connection with R₂₁,

R₂₂₁ to R₂₂₄ are each the same as described herein in connection with R₂₂,

R₂₃₁ to R₂₃₄ are each the same as described herein in connection with R₂₃,

R₂₄₁ to R₂₄₄ are each the same as described herein in connection with R₂₄,

R₂₅₁ to R₂₅₅ are each the same as described herein in connection with R₂₅,

R₂₆₁ to R₂₆₄ are each the same as described herein in connection with R₂₆,

R₂₇₁ to R₂₇₄ are each the same as described herein in connection with R₂₇, and

X₁ to X₃ are respectively the same as described herein in connection with X₁ to X₃ in Formulae 2.

In Formulae 2-1 to 2-5, R₂₁₁ to R₂₁₄, R₂₂₁ to R₂₂₄, R₂₃₁ to R₂₃₄, R₂₄₁ to R₂₄₄, R₂₅₁ to R₂₅₅, R₂₆₁ to R₂₆₄, and R₂₇₁ to R₂₇₄ may each independently be hydrogen or deuterium.

In one or more embodiments, the first compound may include at least one deuterium, the second compound may include at least one deuterium, or each of the first compound and the second compound may include at least one deuterium.

In one or more embodiments, the first compound may include at least four deuteriums, the second compound may include at least four deuteriums, or each of the first compound and the second compound may include at least four deuteriums.

In one or more embodiments, the transition metal-containing compound may be a compound represented by Formula 3:

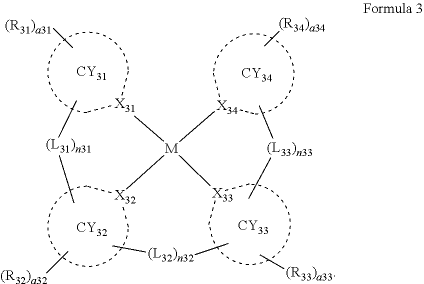

In Formula 3, M may be platinum (Pt), palladium (Pd), copper (Cu), silver (Ag), gold (Au), rhodium (Rh), ruthenium (Ru), osmium (Os), titanium (Ti), zirconium (Zr), hafnium (Hf), europium (Eu), terbium (Tb), or thulium (Tm).

In one or more embodiments, M may be Pt.

X₃₁ to X₃₄ in Formula 3 may each independently be C or N.

In one or more embodiments, X₃₁ may be C. In one or more embodiments, X₃₁ in Formula 3 may be C, and C may be a carbon atom of a carbene moiety.

In one or more embodiments, X₃₁ in Formula 3 may be N.

In one or more embodiments, X₃₂ and X₃₃ may each be C, and X₃₄ may be N.

In Formula 3, i) a bond between X₃₁ and M may be a coordinate bond, ii) one selected from among a bond between X₃₂ and M, a bond between X₃₃ and M, and a bond between X₃₄ and M may be a coordinate bond, and the other two may each be a covalent bond.

For example, a bond between X₃₁ and M and a bond between X₃₄ and M may each be a coordinate bond, and a bond between X₃₂ and M and a bond between X₃₃ and M may each be a covalent bond.

In one or more embodiments, X₃₁ may be C, and a bond between X₃₁ and M may be a coordinate bond.

Ring CY₃₁ to ring CY₃₄ in Formula 3 may each independently be a C₅-C₃₀ carbocyclic group or a C₁-C₃₀ heterocyclic group.

For example, ring CY₃₁ may be a nitrogen-containing C₁-C₆₀ heterocyclic group.

Ring CY₃₁ in Formula 3 may be i) an X₁-containing 5-membered ring, ii) an X₃₁-containing 5-membered ring to which at least one 6-membered ring is condensed, or iii) an X₃₁-containing 6-membered ring. In one or more embodiments, ring CY₃₁ in Formula 3 may be i) an X₃₁-containing 5-membered ring or ii) an X₃₁-containing 5-membered ring to which at least one 6-membered ring is condensed. For example, ring CY₃₁ may include a 5-membered ring bonded to M in Formula 3 via X₃₁. Here, the X₃₁-containing 5-membered ring may be a pyrrole group, a pyrazole group, an imidazole group, a triazole group, an oxazole group, an iso-oxazole group, a thiazole group, an isothiazole group, an oxadiazole group, or a thiadiazole group, and the X₃₁-containing 6-membered ring and the 6-membered ring which may be optionally condensed to the X₃₁-containing 5-membered ring may each independently be a benzene group, a pyridine group, or a pyrimidine group.

In one or more embodiments, ring CY₃₁ may be an X₃₁-containing 5-membered ring, and the X₃₁-containing 5-membered ring may be an imidazole group or a triazole group.

In one or more embodiments, ring CY₃₁ may be an X₃₁-containing 5-membered ring to which at least one 6-membered ring is condensed, and the X₃₁-containing 5-membered ring to which the at least one 6-membered ring is condensed may be a benzimidazole group or an imidazopyridine group.

In one or more embodiments, ring CY₃₁ may be an imidazole group, a triazole group, a benzimidazole group, or an imidazopyridine group.

In one or more embodiments, X₃₁ may be C, and ring CY₃₁ may be an imidazole group, a triazole group, a benzimidazole group, a naphthoimidazol group, or an imidazopyridine group.

In one or more embodiments, ring CY₃₂ may be a benzene group, a pyridine group, a pyrimidine group, a naphthalene group, a dibenzofuran group, a dibenzothiophene group, a carbazole group, a fluorene group, a dibenzosilole group, a naphthobenzofuran group, a naphthobenzothiophene group, a benzocarbazole group, a benzofluorene group, a naphthobenzosilole group, a dinaphthofuran group, a dinaphthothiophene group, a dibenzocarbazole group, a dibenzofluorene group, a dinaphthosilole group, an azadibenzofuran group, an azadibenzothiophene group, an azacarbazole group, an azafluorene group, an azadibenzosilole group, an azanaphthobenzofuran group, an azanaphthobenzothiophene group, an azabenzocarbazole group, an azabenzofluorene group, an azanaphthobenzosilole group, an azadinaphthofuran group, an azadinaphthothiophene group, an azadibenzocarbazole group, an azadibenzofluorene group, or an azadinaphthosilole group.

In one or more embodiments, ring CY₃₂ may be a benzene group, a pyridine group, a pyrimidine group, a naphthalene group, a dibenzofuran group, a dibenzothiophene group, a carbazole group, a fluorene group, or a dibenzosilole group.

In Formula 3, ring CY₃₃ may be: a C₂-C₈ monocyclic group; or a C₄-C₂₀ polycyclic group in which two or three C₂-C₈ monocyclic groups are condensed with each other.

For example, in Formula 3, ring CY₃₃ may be: a C₄-C₆ monocyclic group; or a C₆-C₁₄ polycyclic group in which two or three C₄-C₆ monocyclic groups are condensed with each other.

Throughout the specification, a C₂-C₈ monocyclic group may refer to a non-condensed ring group, and may be, for example, a cyclopentadiene group, a pyrrole group, a pyrazole group, an imidazole group, a triazole group, an oxazole group, an isooxazole group, a thiazole group, an isothiazole group, an oxadiazole group, a thiadiazole group, a benzene group, a pyridine group, a pyrimidine group, a pyrazine group, a pyridazine group, a triazine group, a cycloheptadiene group, a cyclooctadiene group, and/or the like.

For example, ring CY₃₃ may be a benzene group, a pyridine group, a pyrimidine group, a naphthalene group, a dibenzofuran group, a dibenzothiophene group, a carbazole group, a fluorene group, a dibenzosilole group, an azadibenzofuran group, an azadibenzothiophene group, an azacarbazole group, an azafluorene group, or an azadibenzosilole group.

In Formula 3, ring CY₃₄ may be a nitrogen-containing C₁-C₆₀ heterocyclic group.

For example, ring CY₃₄ may be a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, a benzopyrazole group, a benzimidazole group, or a benzothiazole group.

In Formula 3, L₃₁ to L₃₃ may each independently be a single bond, *—C(R₁ₐ)(R_{1b})—*′, *—C(R₁ₐ)=*′, *═C(R₁ₐ)—*′, *—C(R₁ₐ)═C(R_{1b})—*′, *—C(═O)—*′, *—C(═S)—*′, *—C≡C—*′, *—B(R₁ₐ)—*′, *—N(R₁ₐ)—*′, *—O—*′, *—P(R₁ₐ)—*′, *—Si(R₁ₐ)(R_{1b})—*′, *—P(═O)(R₁ₐ)—*′, *—S—*′, *—S(═O)—*′, *—S(═O)₂—*′, or *—Ge(R₁ₐ)(R_{1b})—*′, and * and *′ may each indicate a binding site to a neighboring atom.

R₁ₐ and R_{1b} are respectively the same as described herein.

In one or more embodiments, L₃₁ and L₃₃ may each be a single bond, and L₃₂ may be *—C(R₁ₐ)(R_{1b})—*′, *—B(R₁ₐ)—*′, *—N(R₁ₐ)—*′, *—O—*′, *—P(R₁ₐ)—*′, *—Si(R₁ₐ)(R_{1b})—*, or *—S—*′.

In one or more embodiments, L₃₂ may be *—O—*′ or *—S—*′.

In Formula 3, n31, n32, and n33 indicate the number of L₃₁(s), the number L₃₂(s), and the number of L₃₃(s), respectively, and may each independently be an integer from 1 to 5. When each of n31 to n33 is 2 or more, two or more of each of L₃₁ to L₃₃ may be identical to, or different from, each other.

In one or more embodiments, n32 may be 1.

R₃₁ to R₃₄, R₁ₐ, and R_{1b} in Formula 3 and L₃₁ to L₃₃ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

R₁₀ₐ and Q₁ to Q₃ may respectively be the same as described herein.

In one or more embodiments, R₃₁ to R₃₄, R₁ₐ, and R_{1b} may each independently be:

hydrogen, deuterium, —F, —Cl, —Br, —I, a hydroxyl group, a cyano group, a nitro group, a C₁-C₂₀ alkyl group, or a C₁-C₂₀ alkoxy group;

a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzothiazolyl group, a benzoisoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, —O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or any combination thereof; or

—C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

In one or more embodiments, Q₁ to Q₃ and Q₃₁ to Q₃₃ may each be the same as described herein.

In one or more embodiments, R₃₁ to R₃₄, R₁ₐ, and R_{1b} may each independently be:

hydrogen, deuterium, —F, —Cl, —Br, —I, or a C₁-C₂₀ alkyl group;

a C₁-C₂₀ alkyl group unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof; or

a phenyl group, a biphenyl group, a terphenyl group, a C₁-C₁₀ alkylphenyl group, or a naphthyl group, each unsubstituted or substituted with deuterium, —F, —Cl, —Br, —I, —CD₃, —CD₂H, —CDH₂, —CF₃, —CF₂H, —CFH₂, a C₁-C₂₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, a C₁-C₁₀ alkylphenyl group, or any combination thereof.

In Formula 3, a31, a32, a33, and a34 indicate the number of R₃₁(s), the number R₃₂(s), the number R₃₃(s), and the number of R₃₄(s), respectively, and may each independently be an integer from 1 to 10. When each of a31 to a34 is 2 or more, two or more of each of R₃₁ to R₃₄ may be identical to or different from each other.

In reference to Formulae 502 and 503, R₅₀₀ₐ, R_{500b}, R₅₀₁ to R₅₀₈, R₅₀₅ₐ, R_{505b}, R₅₀₆ₐ, R_{506b}, R₅₀₇ₐ, R_{507b}, R₅₀₈ₐ, and R_{508b} may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂). Q₁ to Q₃ may each be the same as described herein.

For example, in Formulae 502 and 503, R₅₀₀ₐ, R_{500b}, R₅₀₁ to R₅₀₈, R₅₀₅ₐ, R_{505b}, R₅₀₆ₐ, R_{506b}, R₅₀₇ₐ, R_{507b}, R₅₀₈ₐ, and R_{508b} may each independently be:

hydrogen, deuterium, —F, —Cl, —Br, —I, a hydroxyl group, a cyano group, a nitro group, a C₁-C₂₀ alkyl group, or a C₁-C₂₀ alkoxy group;

a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isooxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, an azadibenzosilolyl group, or a group represented by Formula 91, each unsubstituted or substituted with deuterium, —F, —Cl, —Br, —I,-CD₃,-CD₂H,-CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isooxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzothiazolyl group, a benzoisoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, a —O(Q₃₁), a —S(Q₃₁), a —Si(Q₃₁)(Q₃₂)(Q₃₃), a —N(Q₃₁)(Q₃₂), a —B(Q₃₁)(Q₃₂), a —P(Q₃₁)(Q₃₂), a —C(═O)(Q₃₁), a —S(═O)₂(Q₃₁), a —P(═O)(Q₃₁)(Q₃₂), or any combination thereof; or

—C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and

Q₁ to Q₃ and Q₃₁ to Q₃₃ may each independently be:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or

an n-propyl group, an iso-propyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, a pyridinyl group, a pyrimidinyl group, a pyridazinyl group, a pyrazinyl group, or a triazinyl group, each unsubstituted or substituted with deuterium, a C₁-C₁₀ alkyl group, a phenyl group, a biphenyl group, a pyridinyl group, a pyrimidinyl group, a pyridazinyl group, a pyrazinyl group, a triazinyl group, or any combination thereof.

### Examples of Compounds

In one or more embodiments, the first compound may be selected from among Compounds H1 to H20, and the second compound may be selected from among Compounds E1 to E10:

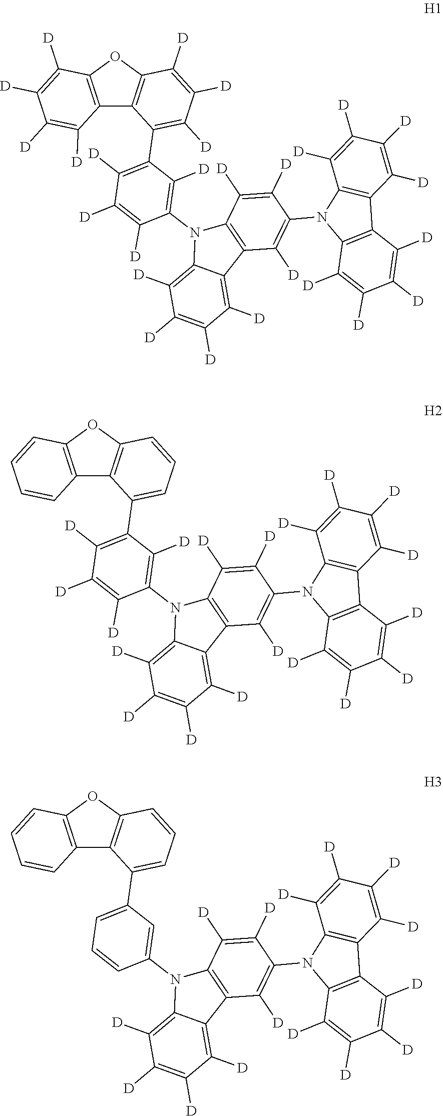

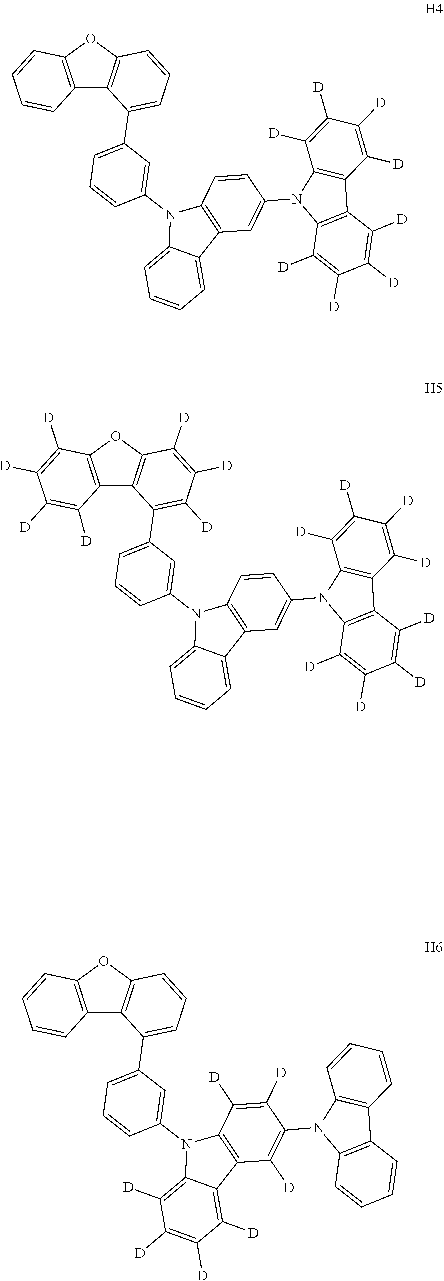 [SMILES: [2H]c1c([2H])c([2H])c2c(c1[2H])c1c([2H])c([2H])c([2H])c([2H])c1n2-c1ccc2c(c1)c1ccccc1n2-c1cccc(-c2cccc3oc4ccccc4c23)c1 ; [2H]c1c([2H])c([2H])c2c(oc3c([2H])c([2H])c([2H])c(-c4cccc(-n5c6ccccc6c6cc(-n7c8c([2H])c([2H])c([2H])c([2H])c8c8c([2H])c([2H])c([2H])c([2H])c87)ccc65)c4)c32)c1[2H] ; [2H]c1c([2H])c([2H])c2c(c1[2H])c1c([2H])c(-n3c4ccccc4c4ccccc43)c([2H])c([2H])c1n2-c1cccc(-c2cccc3oc4ccccc4c23)c1]

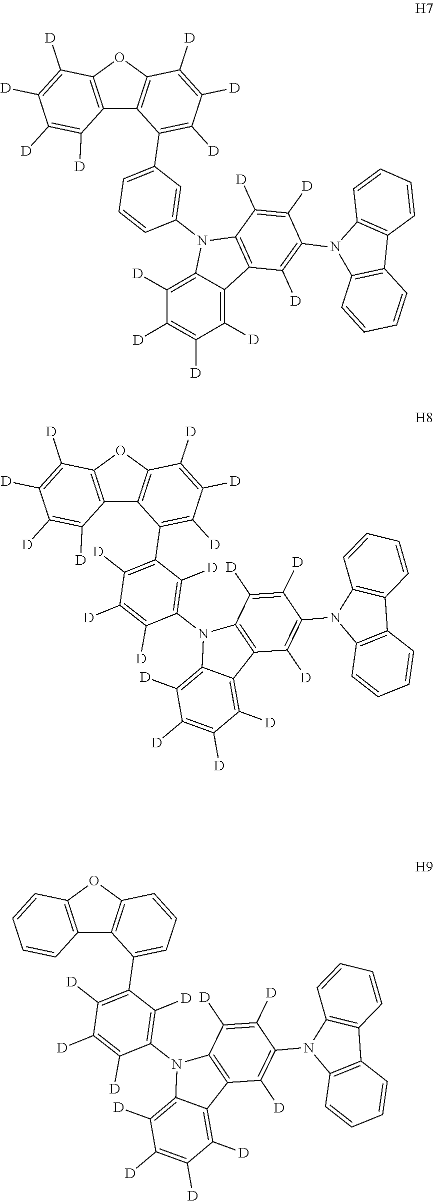 [SMILES: [2H]c1c([2H])c([2H])c2c(oc3c([2H])c([2H])c([2H])c(-c4cccc(-n5c6c([2H])c([2H])c([2H])c([2H])c6c6c([2H])c(-n7c8ccccc8c8ccccc87)c([2H])c([2H])c65)c4)c32)c1[2H] ; [2H]c1c([2H])c(-c2c([2H])c([2H])c([2H])c3oc4c([2H])c([2H])c([2H])c([2H])c4c23)c([2H])c(-n2c3c([2H])c([2H])c([2H])c([2H])c3c3c([2H])c(-n4c5ccccc5c5ccccc54)c([2H])c([2H])c32)c1[2H] ; [2H]c1c([2H])c(-c2cccc3oc4ccccc4c23)c([2H])c(-n2c3c([2H])c([2H])c([2H])c([2H])c3c3c([2H])c(-n4c5ccccc5c5ccccc54)c([2H])c([2H])c32)c1[2H]]

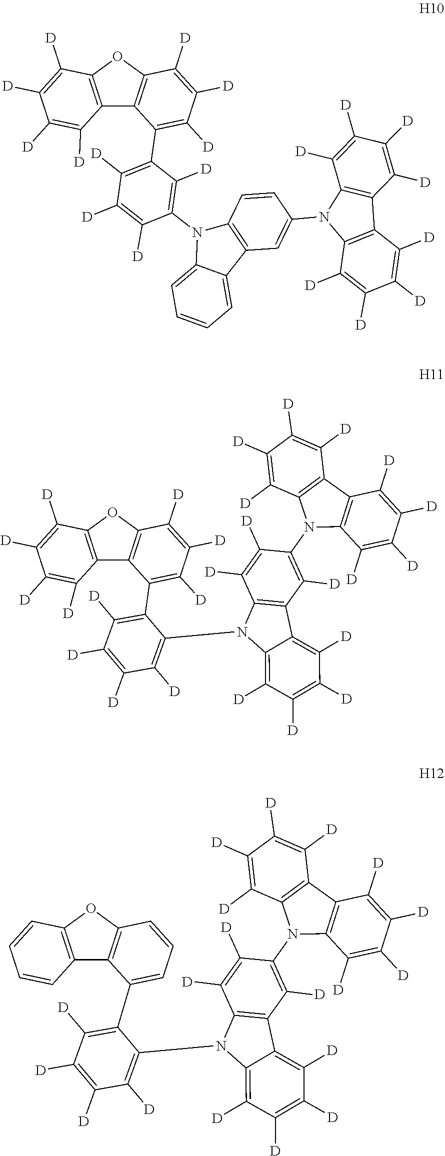

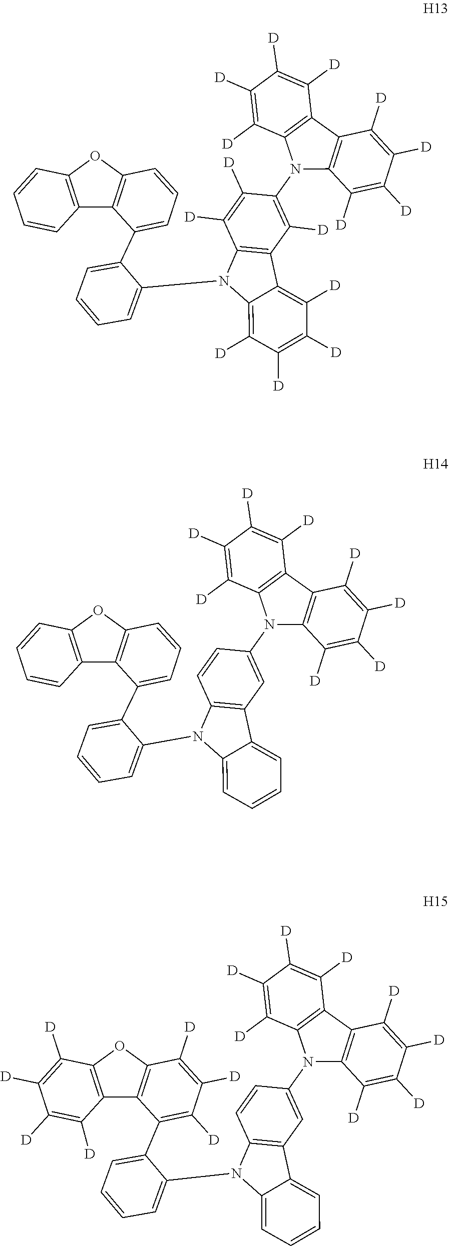 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1cccc2oc3ccccc3c12 ; [2H]c1c([2H])c([2H])c2c(c1[2H])c1c([2H])c([2H])c([2H])c([2H])c1n2-c1ccc2c(c1)c1ccccc1n2-c1ccccc1-c1cccc2oc3ccccc3c12 ; [2H]c1c([2H])c([2H])c2c(oc3c([2H])c([2H])c([2H])c(-c4ccccc4-n4c5ccccc5c5cc(-n6c7c([2H])c([2H])c([2H])c([2H])c7c7c([2H])c([2H])c([2H])c([2H])c76)ccc54)c32)c1[2H]]

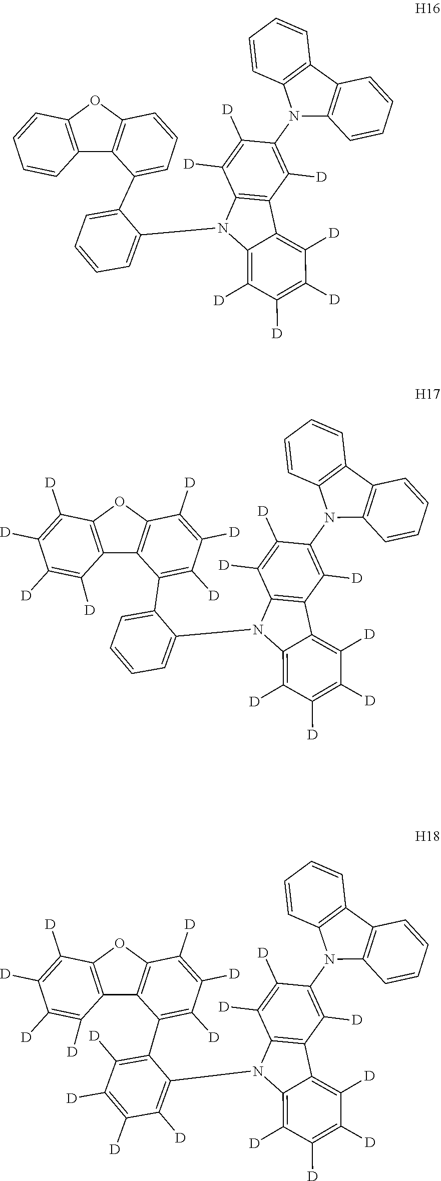 [SMILES: [2H]c1c([2H])c([2H])c2c(c1[2H])c1c([2H])c(-n3c4ccccc4c4ccccc43)c([2H])c([2H])c1n2-c1ccccc1-c1cccc2oc3ccccc3c12 ; [2H]c1c([2H])c([2H])c2c(oc3c([2H])c([2H])c([2H])c(-c4ccccc4-n4c5c([2H])c([2H])c([2H])c([2H])c5c5c([2H])c(-n6c7ccccc7c7ccccc76)c([2H])c([2H])c54)c32)c1[2H] ; [2H]c1c([2H])c([2H])c(-n2c3c([2H])c([2H])c([2H])c([2H])c3c3c([2H])c(-n4c5ccccc5c5ccccc54)c([2H])c([2H])c32)c(-c2c([2H])c([2H])c([2H])c3oc4c([2H])c([2H])c([2H])c([2H])c4c23)c1[2H]]

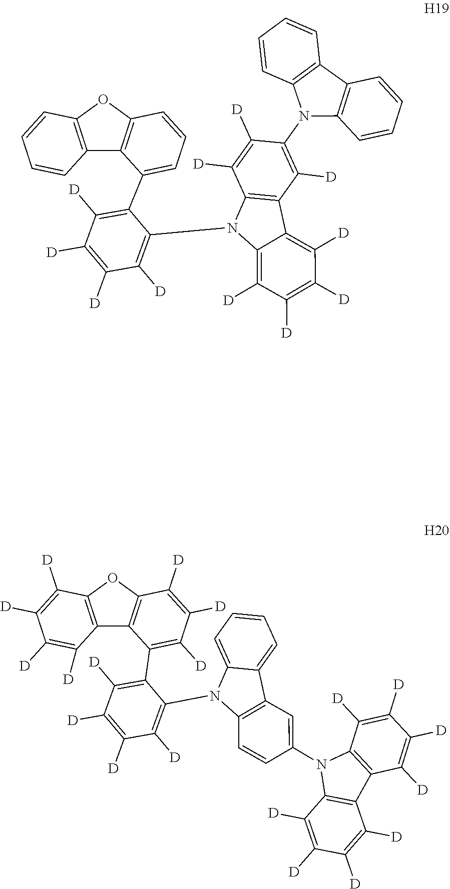 [SMILES: [2H]c1c([2H])c([2H])c(-n2c3c([2H])c([2H])c([2H])c([2H])c3c3c([2H])c(-n4c5ccccc5c5ccccc54)c([2H])c([2H])c32)c(-c2cccc3oc4ccccc4c23)c1[2H] ; [2H]c1c([2H])c([2H])c(-n2c3ccccc3c3cc(-n4c5c([2H])c([2H])c([2H])c([2H])c5c5c([2H])c([2H])c([2H])c([2H])c54)ccc32)c(-c2c([2H])c([2H])c([2H])c3oc4c([2H])c([2H])c([2H])c([2H])c4c23)c1[2H]]

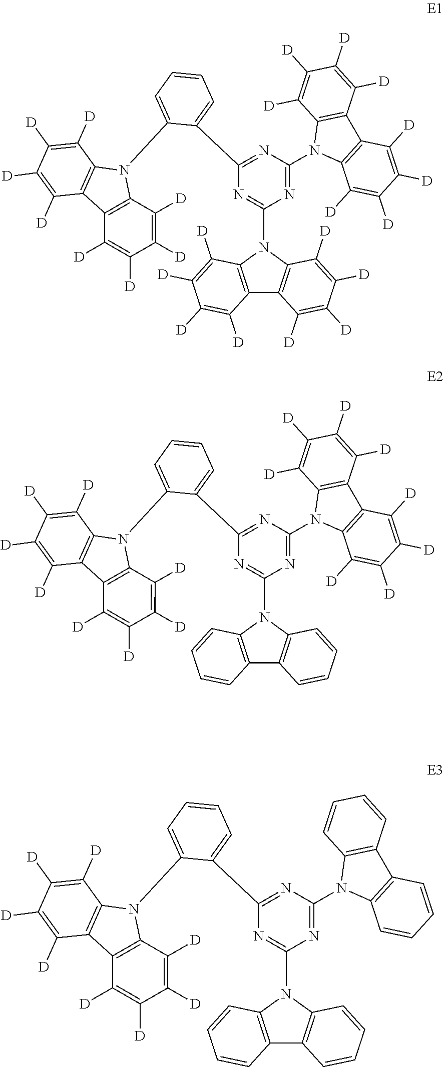

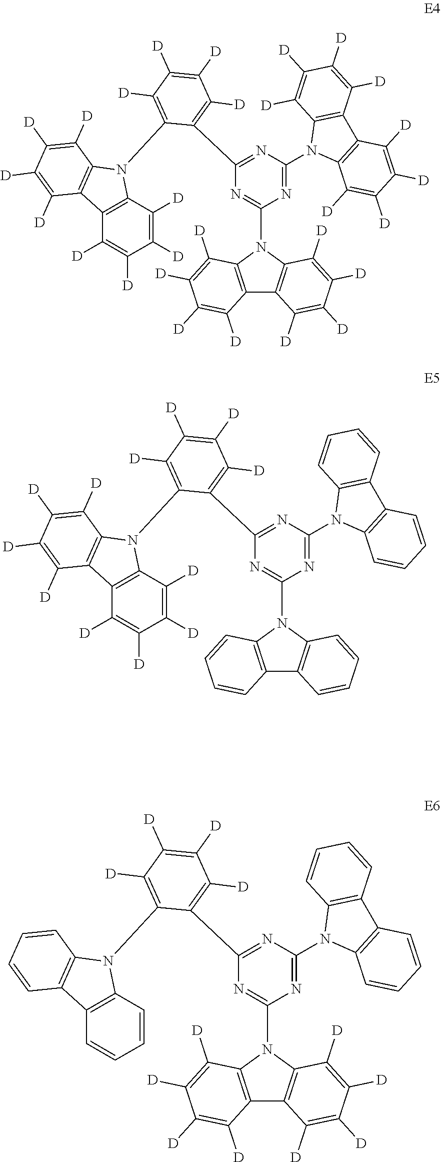

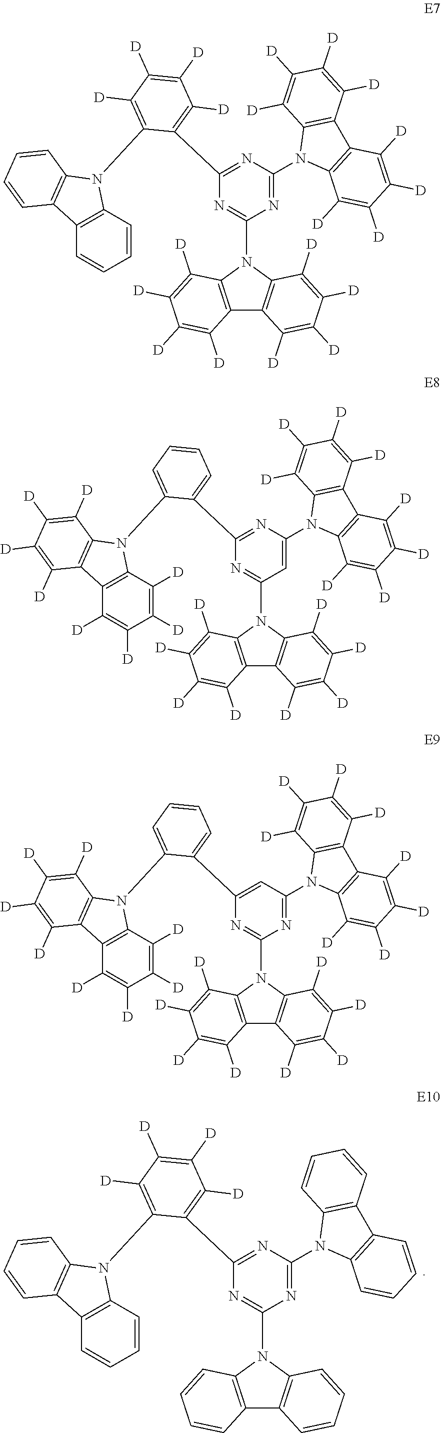

The composition including the first compound and the second compound according to the present disclosure satisfies the aforementioned HOMO energy level, LUMO energy level, or triplet (T1) energy level, and thus has an advantage in luminescence efficiency and energy transmission, and by combining the composition with fluorescent and phosphorescent dopants, the efficiency and lifespan characteristics of the light-emitting device may be enhanced or improved.

In some embodiments, as the first compound and the second compound have high electron transfer characteristics based on a plurality of heteroaryl substituents, the first and second compounds may be utilized as an electron-transporting material in an organic EL device.

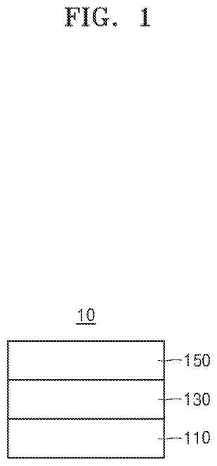

### Description of FIG. 1

FIG. 1 is a schematic cross-sectional view of a light-emitting device 10 according to one or more embodiments. The light-emitting device 10 includes a first electrode 110, an interlayer 130, and a second electrode 150.

Hereinafter, the structure of the light-emitting device 10 according to one or more embodiments and a method of manufacturing the light-emitting device 10 will be described with reference to FIG. 1.

### First Electrode 110

In FIG. 1, a substrate may be additionally arranged under the first electrode 110 or on the second electrode 150. In one or more embodiments, as the substrate, a glass substrate or a plastic substrate may be utilized. In one or more embodiments, the substrate may be a flexible substrate, and may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high work-function material that facilitates injection of holes. The term “high work-function material” as utilized herein refers to a substance (e.g., a metal or metal alloy) that requires a relatively high amount of energy to emit electrons from its surface.

The first electrode 110 may be a reflective electrode, a semi-transmissive electrode, or a transmissive electrode. In one or more embodiments, when the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layer structure consisting of a single layer or a multi-layer structure including multiple layers. For example, the first electrode 110 may have a three-layer structure of ITO/Ag/ITO.

### Interlayer 130

The interlayer 130 is arranged on the first electrode 110. The interlayer 130 may include the emission layer.

The interlayer 130 may further include a hole transport region arranged between the first electrode 110 and the emission layer, and an electron transport region arranged between the emission layer and the second electrode 150.

The interlayer 130 may further include, in addition to one or more suitable organic materials, a metal-containing compound such as a transition metal-containing compound, an inorganic material such as a quantum dot, and/or the like.

In one or more embodiments, the interlayer 130 may include i) two or more emitting units sequentially stacked between the first electrode 110 and the second electrode 150, and ii) a charge generation layer located between two neighboring emitting units. When the interlayer 130 includes the two or more emitting units and the charge generation layer, the light-emitting device 10 may be, or may be referred to as, a tandem light-emitting device.

### Hole Transport Region in Interlayer 130

The hole transport region may have: i) a single-layer structure including (e.g., consisting of) a single layer including (e.g., consisting of) a single material, ii) a single-layer structure including (e.g., consisting of) a single layer including (e.g., consisting) of multiple materials that are different from each other, or iii) a multi-layer structure including (e.g., consisting of) multiple materials including (e.g., consisting of) multiple materials that are different from each other.

The hole transport region may include a hole injection layer, a hole transport layer, an emission auxiliary layer, an electron blocking layer, or any combination thereof.

For example, the hole transport region may have a multi-layer structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure. In one or more embodiments, the constituent layers of each structure are stacked sequentially from the first electrode 110.

The hole transport region may include a compound represented by Formula 201, a compound represented by Formula 202, and/or any combination thereof:

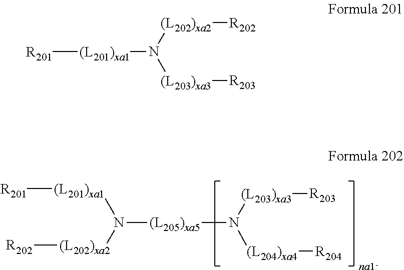

In Formulae 201 and 202, L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,

xa1 to xa4 may each independently be an integer from 0 to 5,

xa5 may be an integer from 1 to 10,

R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group (for example, a carbazole group and/or the like) unsubstituted or substituted with at least one R₁₀ₐ (for example, see Compound HT16),

R₂₀₃ and R₂₀₄ may optionally be linked to each other, via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and

na1 may be an integer from 1 to 4.

For example, each of Formulae 201 and 202 may include at least one of selected from among groups represented by Formulae CY201 to CY217:

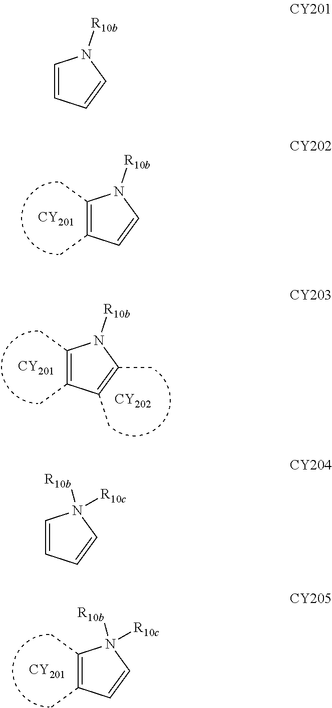

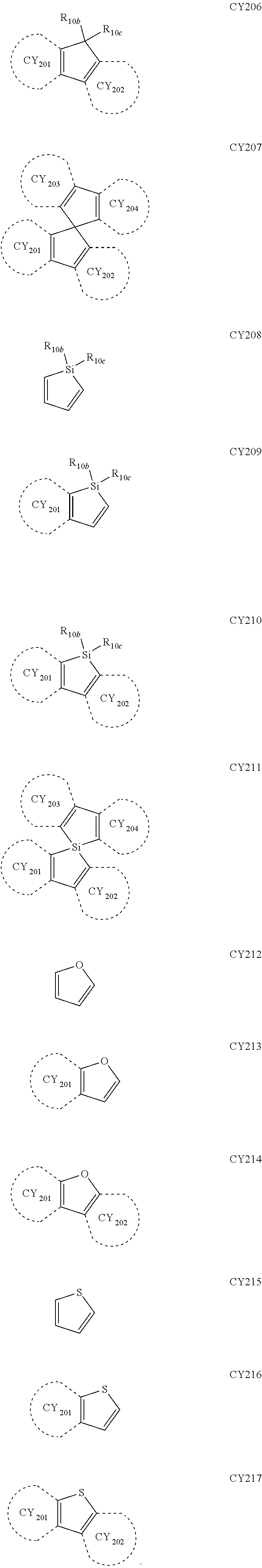

R_{10b} and R_{10c} in Formulae CY₂₀₁ to CY₂₁₇ may each be as defined in R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In one or more embodiments, ring CY₂₀₁ to ring CY₂₀₄ in Formulae CY201 to CY217 may each independently be a benzene group, a naphthalene group, a phenanthrene group, or an anthracene group.

In one or more embodiments, each of Formulae 201 and 202 may include at least one of selected from among the groups represented by Formulae CY201 to CY203.

In one or more embodiments, Formula 201 may include at least one of selected from among the groups represented by Formulae CY201 to CY203 and at least one of the groups represented by Formulae CY204 to CY217.

In one or more embodiments, in Formula 201, xa1 may be 1, R₂₀₁ may be one of the groups represented by Formulae CY201 to CY203, xa2 may be 0, and R₂₀₂ may be one of the groups represented by Formulae CY204 to CY207.

In one or more embodiments, each of Formulae 201 and 202 may not include the (e.g., may exclude any) groups represented by Formulae CY201 to CY203.

In one or more embodiments, each of Formulae 201 and 202 may not include the (e.g., may exclude any) groups represented by Formulae CY201 to CY203, and may include at least one of selected from among the groups represented by Formulae CY204 to CY217.

In one or more embodiments, each of Formulae 201 and 202 may not include (e.g., may exclude) the groups represented by Formulae CY201 to CY217.

In one or more embodiments, the hole transport region may include one selected from among Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), and/or any combination thereof:

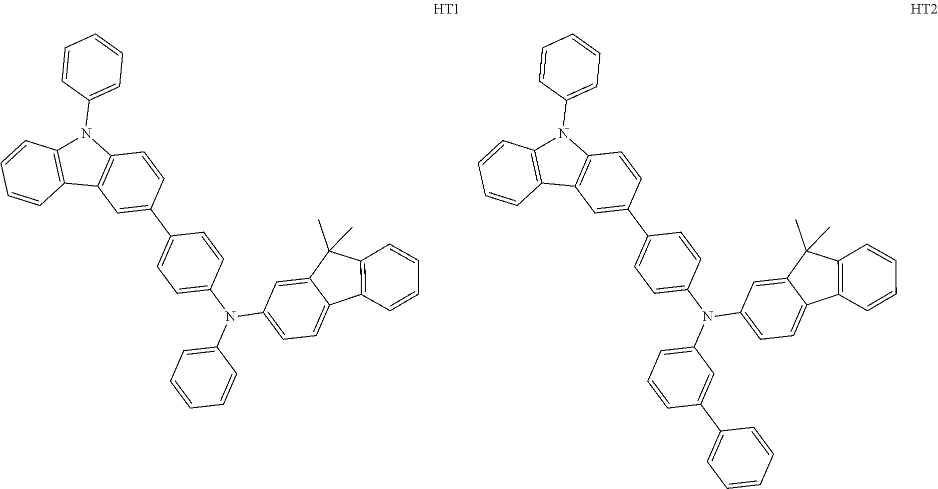 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21]

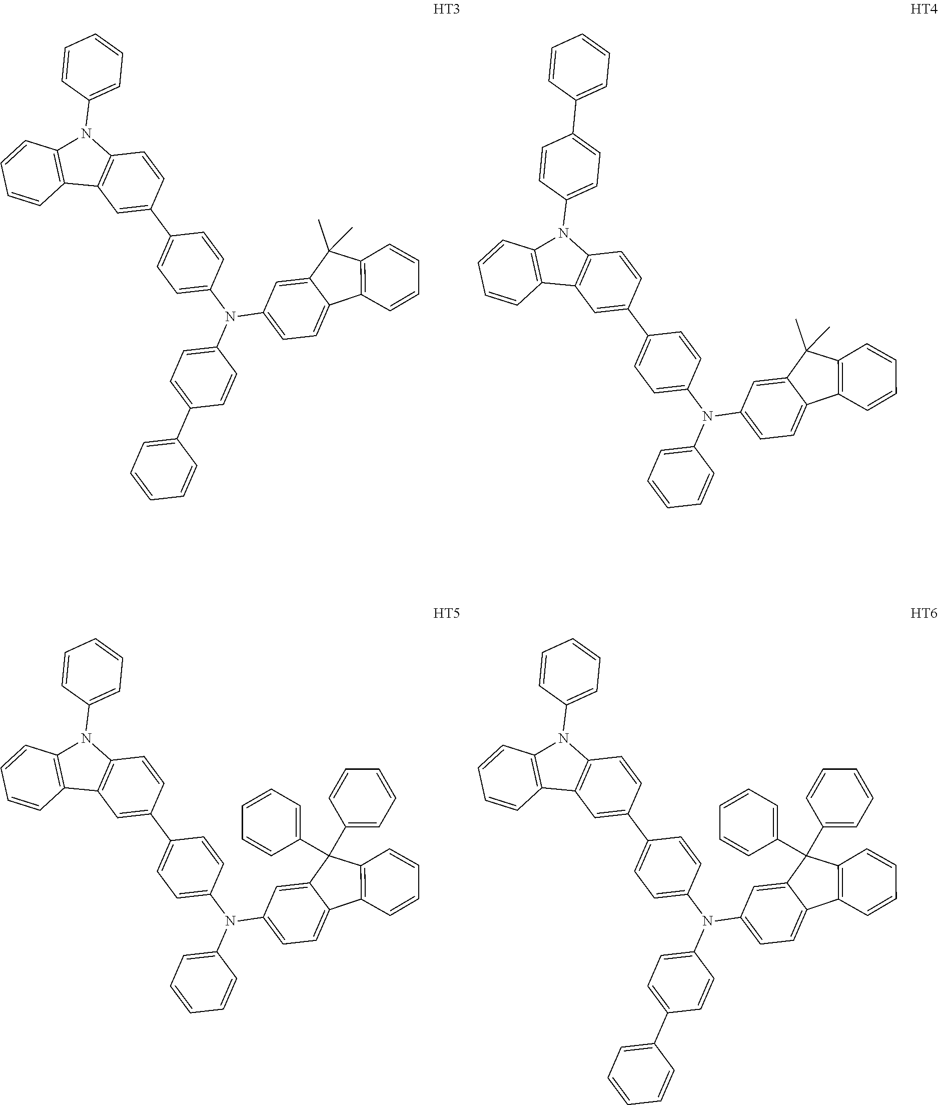 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

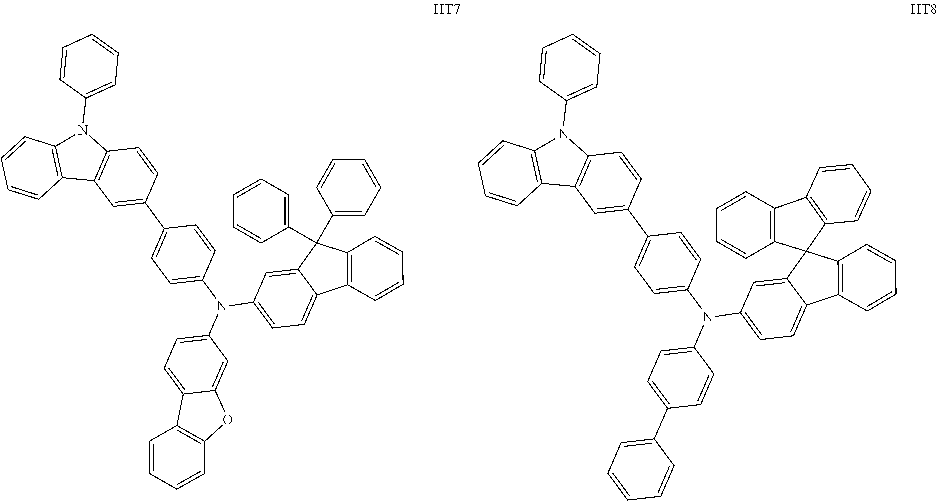 [SMILES: c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

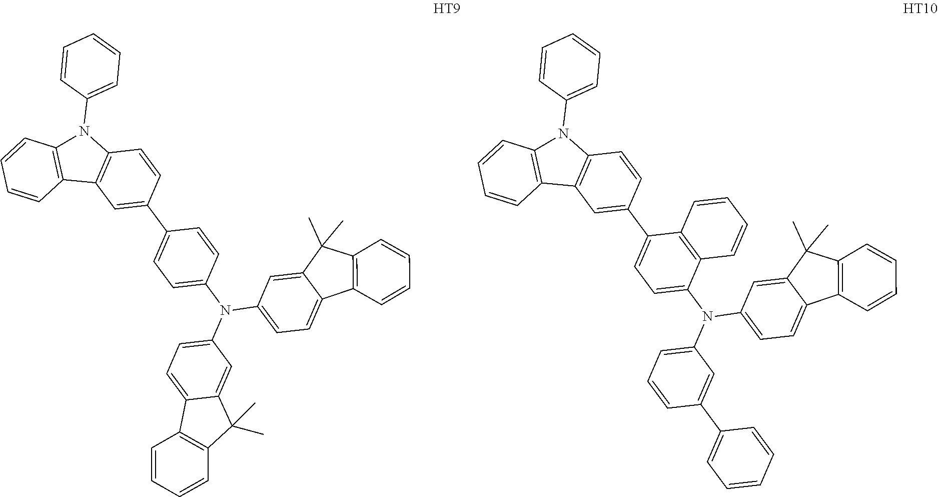 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21]

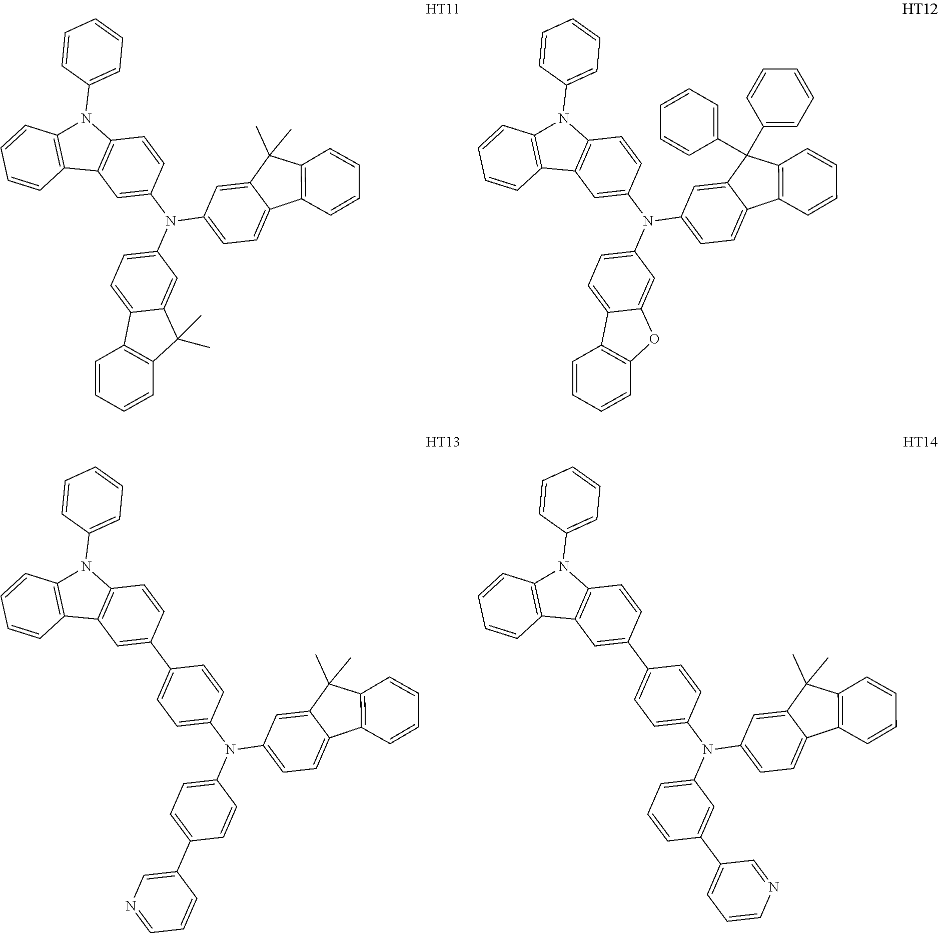 [SMILES: CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1 ; CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21]

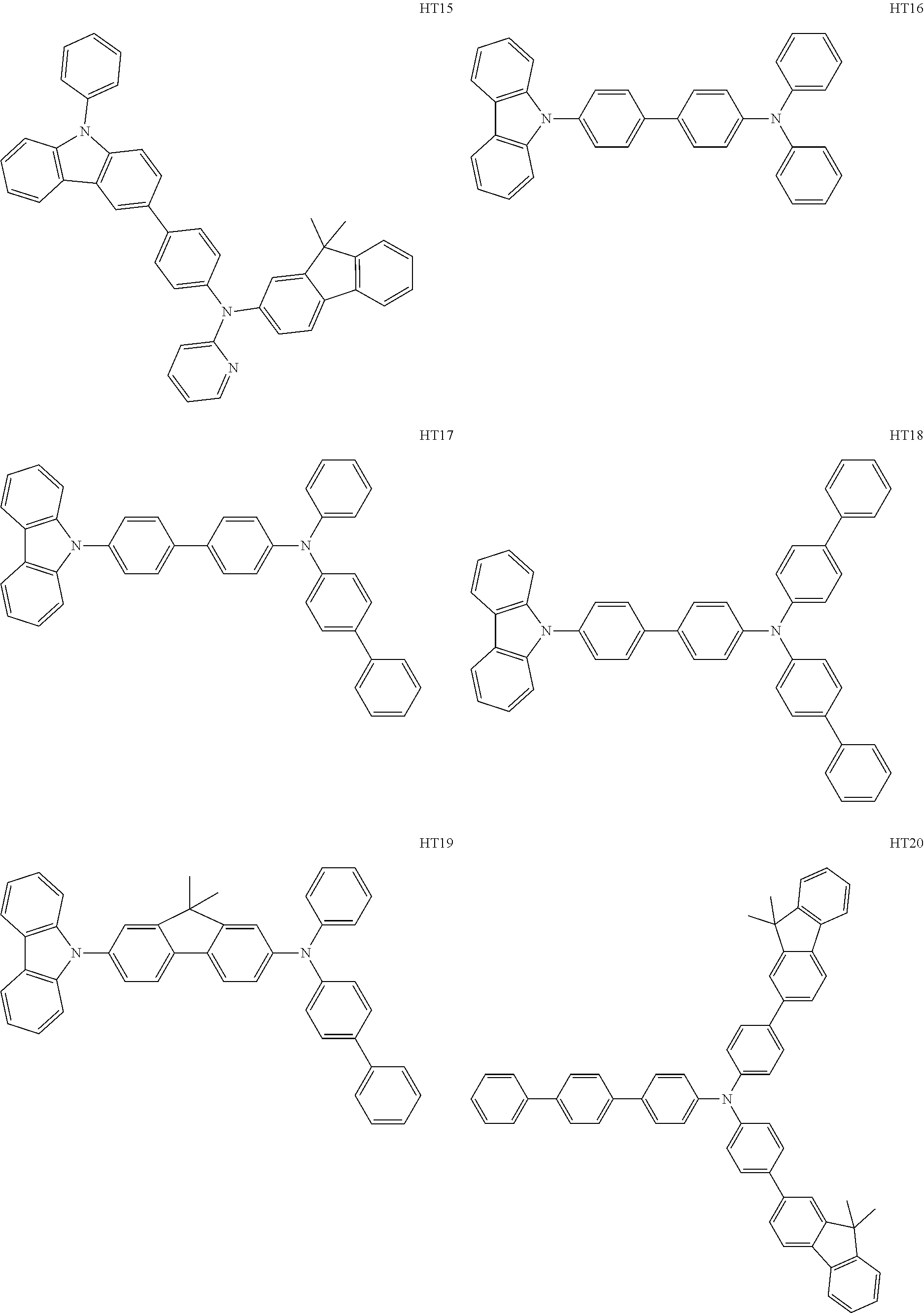 [SMILES: CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1 ; c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

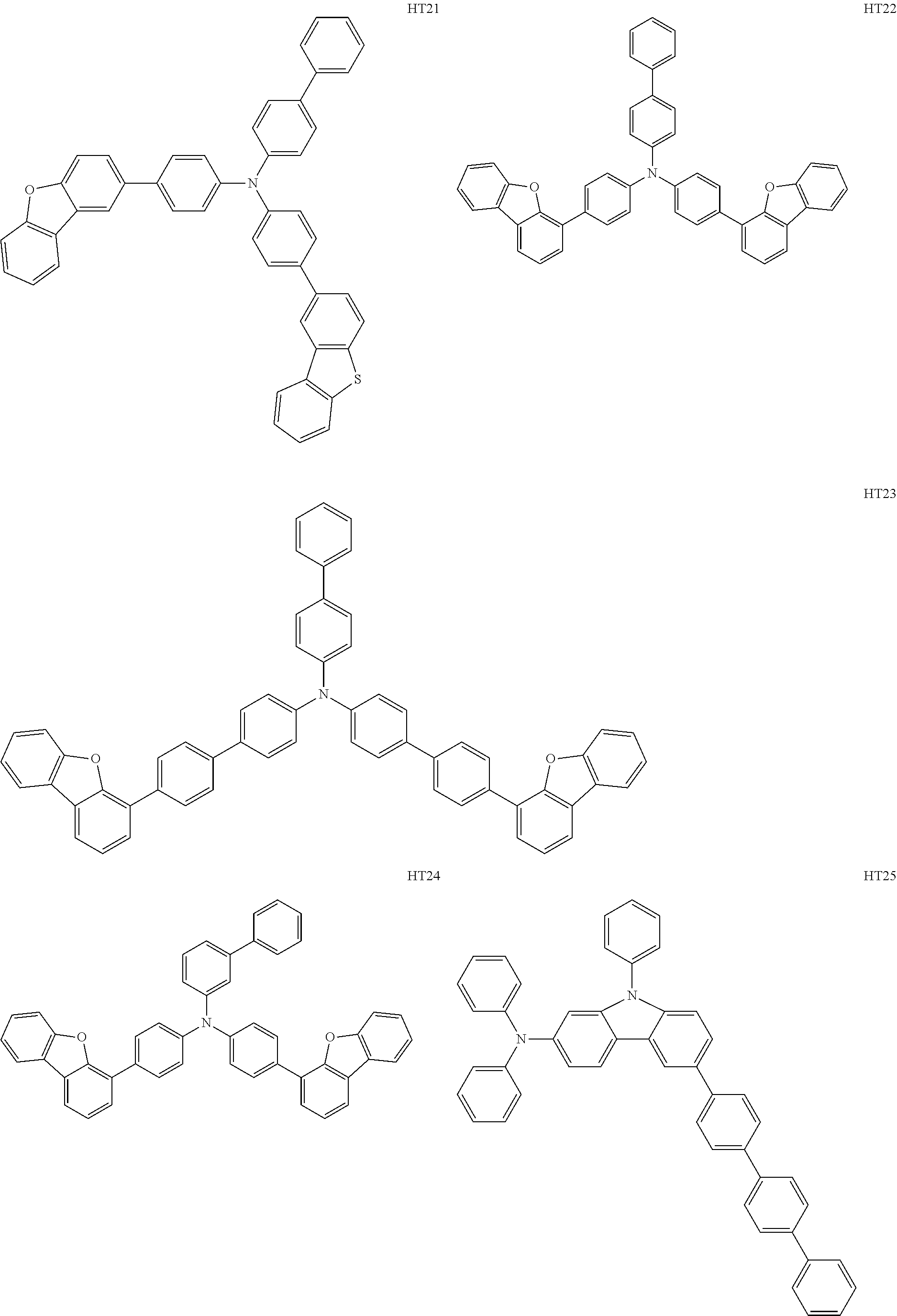 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1 ; c1ccc(-c2cccc(N(c3ccc(-c4cccc5c4oc4ccccc45)cc3)c3ccc(-c4cccc5c4oc4ccccc45)cc3)c2)cc1 ; c1ccc(-c2ccc(-c3ccc(-c4ccc5c(c4)c4ccc(N(c6ccccc6)c6ccccc6)cc4n5-c4ccccc4)cc3)cc2)cc1]

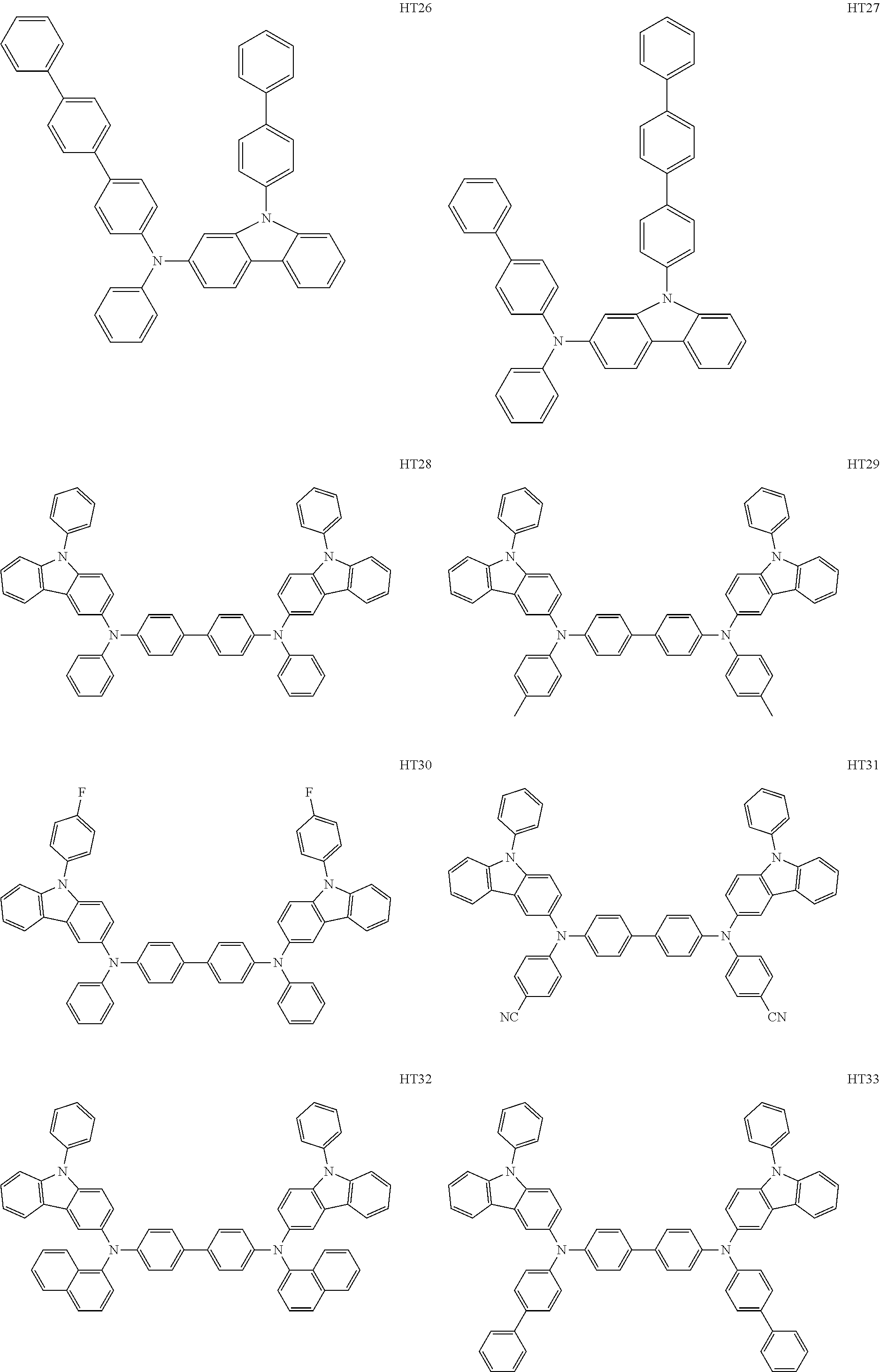

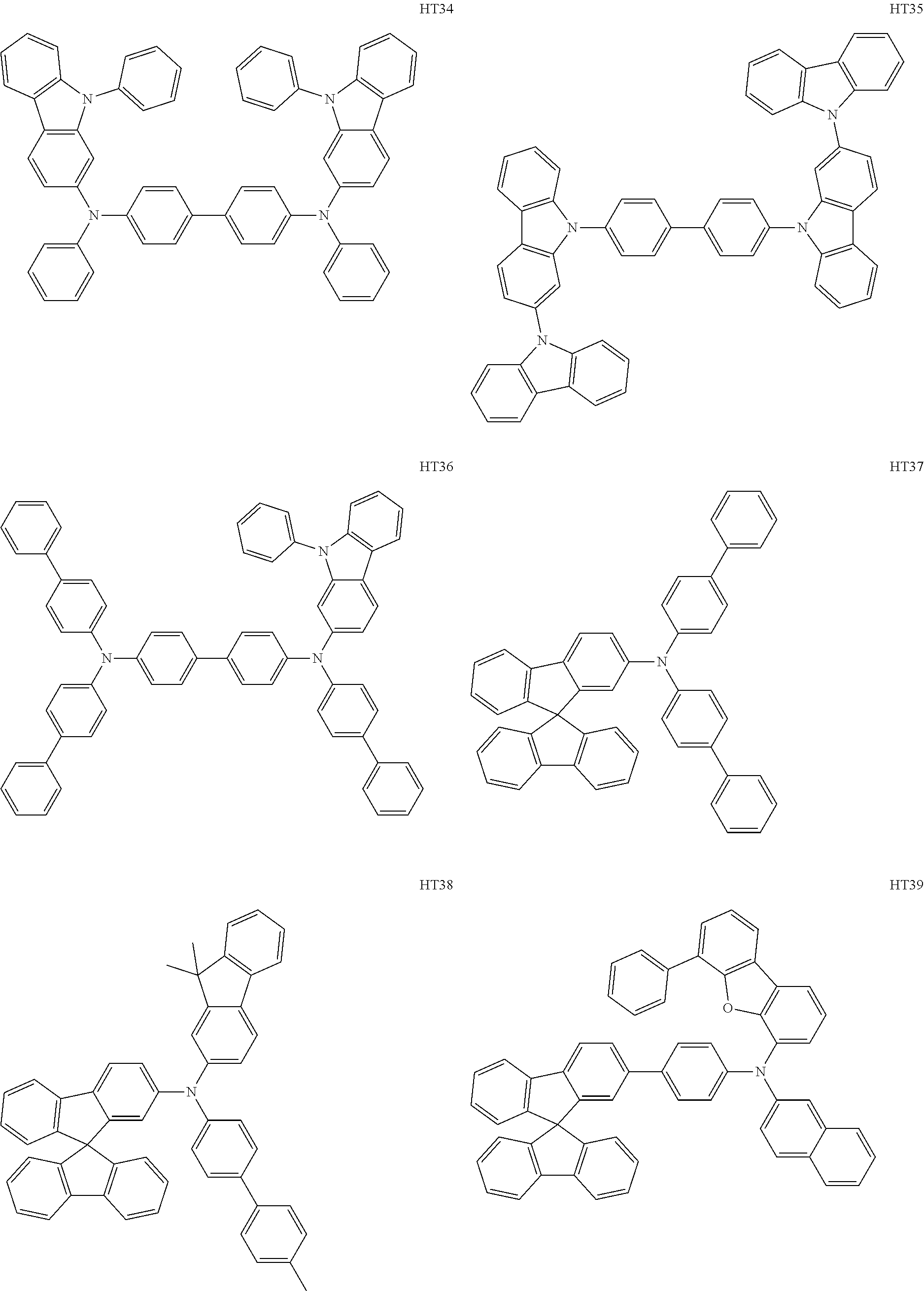

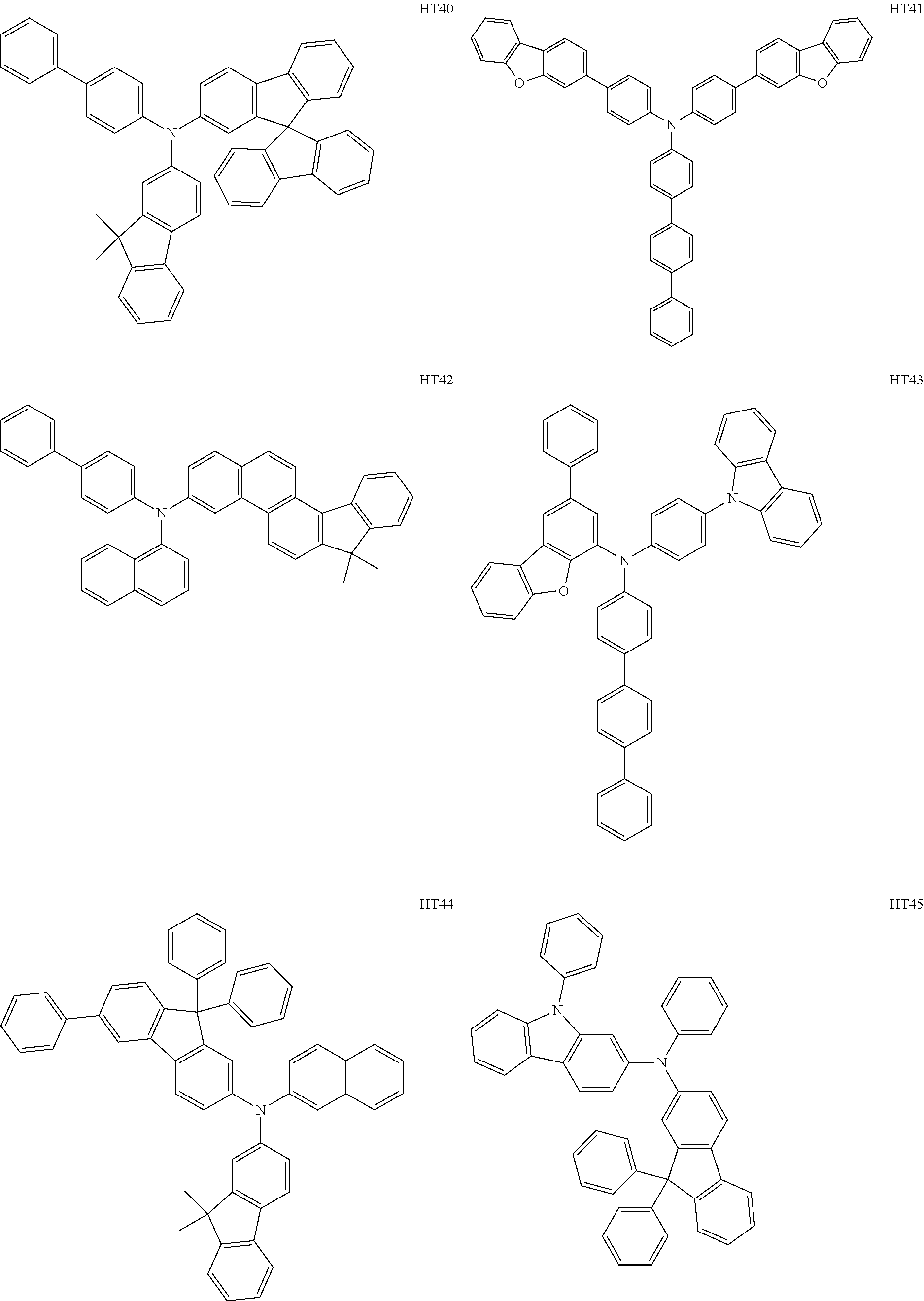

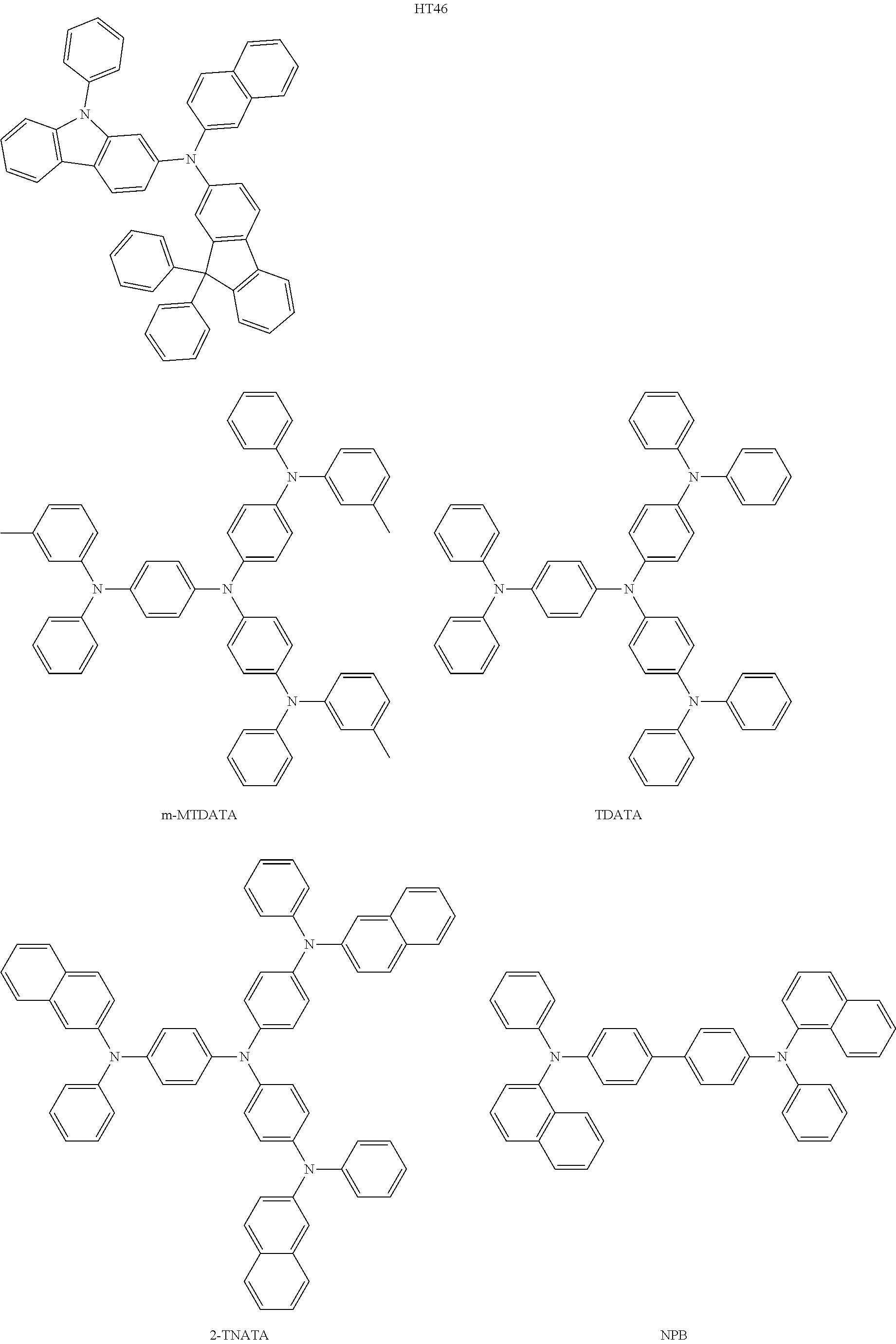

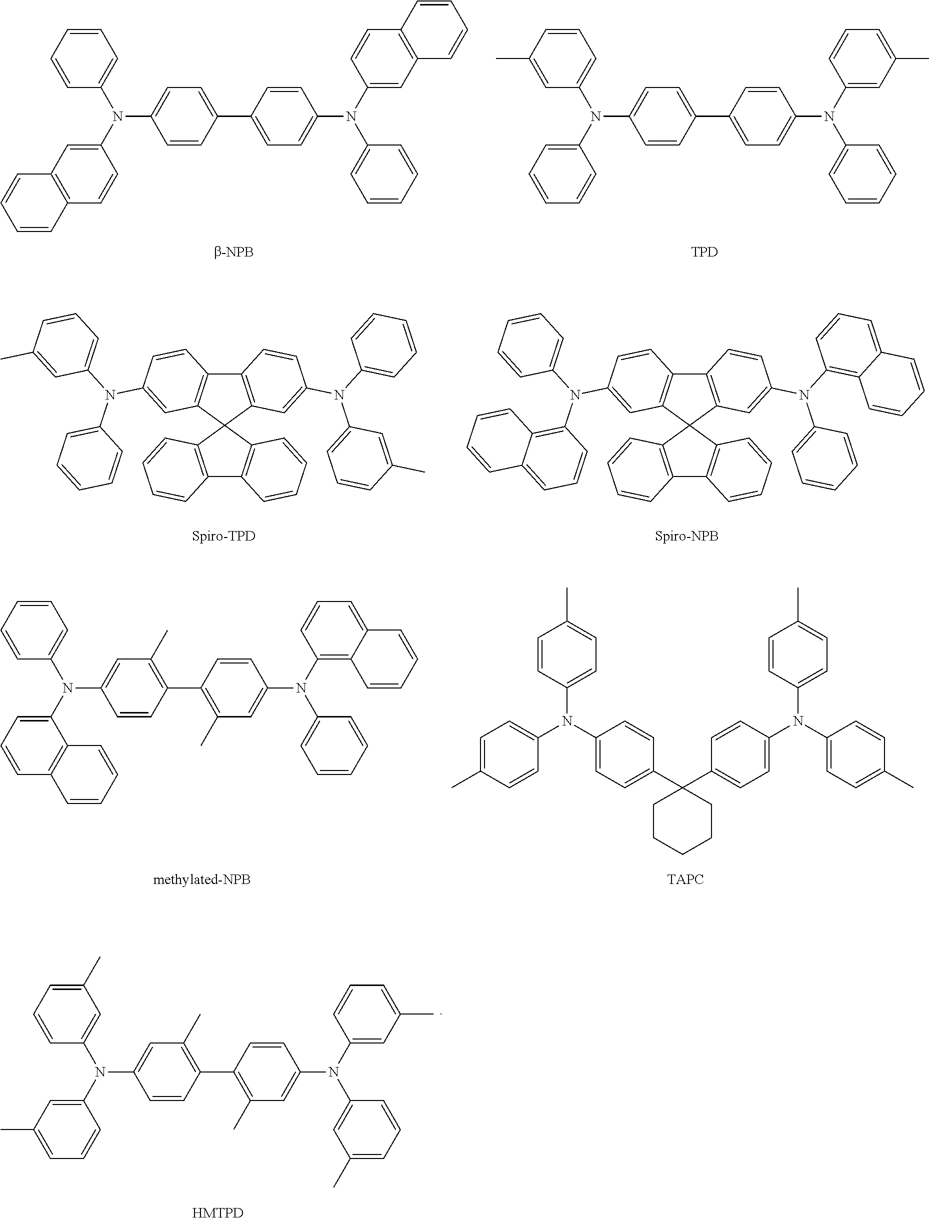

A thickness of the hole transport region may be in a range of about 50 angstrom (Å) to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, satisfactory hole transporting characteristics may be obtained without a substantial increase in driving voltage.

The emission auxiliary layer may increase light-emission efficiency by compensating for an optical resonance distance according to the wavelength of light emitted by the emission layer. In one or more embodiments, the electron blocking layer may block or reduce the leakage of electrons from the emission layer to the hole transport region. Materials that may be included in the hole transport region may be included in the emission auxiliary layer and the electron blocking layer.

p-Dopant

The hole transport region may further include, in addition to these materials, a charge-generation material for the improvement of conductive properties. The charge-generation material may be uniformly or non-uniformly dispersed in the hole transport region (for example, in the form of a single layer consisting of a charge-generation material).

The charge-generation material may be, for example, a p-dopant.

For example, the p-dopant may have a LUMO energy level of less than or equal to about −3.5 eV.

In one or more embodiments, the p-dopant may include a quinone derivative, a cyano group-containing compound, a compound including element EL1 and element EL2, or any combination thereof.

Examples of the quinone derivative are TCNQ, F4-TCNQ, and/or the like.

Examples of the cyano group-containing compound are HAT-CN, a compound represented by Formula 221, and/or the like:

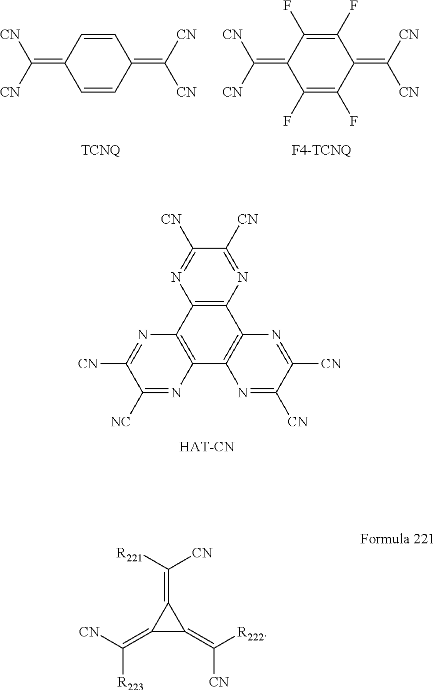

In Formula 221, R₂₂₁ to R₂₂₃ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ, and

at least one of selected from among R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound including element EL1 and element EL2, element EL1 may be metal, metalloid, or any combination thereof, and element EL2 may be non-metal, metalloid, or any combination thereof.

Examples of the metal are an alkali metal (for example, lithium (Li), sodium (Na), potassium (K), rubidium (Rb), cesium (Cs), etc.); an alkaline earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (for example, zinc (Zn), indium (In), tin (Sn), etc.); a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.); and/or the like.

Examples of the metalloid are silicon (Si), antimony (Sb), tellurium (Te), and/or the like.

Examples of the non-metal are oxygen (O), halogen (for example, F, Cl, Br, I, etc.), and/or the like.

Examples of the compound including element EL1 and element EL2 are metal oxide, metal halide (for example, metal fluoride, metal chloride, metal bromide, metal iodide, etc.), metalloid halide (for example, metalloid fluoride, metalloid chloride, metalloid bromide, metalloid iodide, etc.), metal telluride, or any combination thereof.

Examples of the metal oxide are tungsten oxide (for example, WO, W₂O₃, WO₂, WO₃, W₂O₅, etc.), vanadium oxide (for example, VO, V₂O₃, VO₂, V₂O₅, etc.), molybdenum oxide (MoO, Mo₂O₃, MoO₂, MoO₃, Mo₂O₅, etc.), rhenium oxide (for example, ReO₃, etc.), and/or the like.

Examples of the metal halide are alkali metal halide, alkaline earth metal halide, transition metal halide, post-transition metal halide, lanthanide metal halide, and/or the like.

Examples of the alkali metal halide are LiF, NaF, KF, RbF, CsF, LiCl, NaCl, KCl, RbCl, CsCl, LiBr, NaBr, KBr, RbBr, CsBr, LiI, NaI, KI, RbI, CsI, and/or the like.

Examples of the alkaline earth metal halide are BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, BaI₂, and/or the like.

Examples of the transition metal halide are titanium halide (for example, TiF₄, TiC₄, TiBr₄, TiI₄, etc.), zirconium halide (for example, ZrF₄, ZrCl₄, ZrBr₄, ZrI₄, etc.), hafnium halide (for example, HfF₄, HfCl₄, HfBr₄, HfI₄, etc.), vanadium halide (for example, VF₃, VCl₃, VBr₃, VI₃, etc.), niobium halide (for example, NbF₃, NbCl₃, NbBr₃, NbI₃, etc.), tantalum halide (for example, TaF₃, TaCl₃, TaBr₃, TaI₃, etc.), chromium halide (for example, CrF₃, CrCl₃, CrBrs, CrI₃, etc.), molybdenum halide (for example, MoF₃, MoCl₃, MoBr₃, MoI₃, etc.), tungsten halide (for example, WF₃, WCl₃, WBr₃, WI₃, etc.), manganese halide (for example, MnF₂, MnCl₂, MnBr₂, MnI₂, etc.), technetium halide (for example, TcF₂, TcCl₂, TcBr₂, TcI₂, etc.), rhenium halide (for example, ReF₂, ReCl₂, ReBr₂, ReI₂, etc.), iron halide (for example, FeF₂, FeCl₂, FeBr₂, FeI₂, etc.), ruthenium halide (for example, RuF₂, RuCl₂, RuBr₂, RuI₂, etc.), osmium halide (for example, OsF₂, OsC₁₂, OsBr₂, OsI₂, etc.), cobalt halide (for example, CoF₂, CoCl₂, CoBr₂, CoI₂, etc.), rhodium halide (for example, RhF₂, RhCl₂, RhBr₂, RhI₂, etc.), iridium halide (for example, IrF₂, IrCl₂, IrBr₂, IrI₂, etc.), nickel halide (for example, NiF₂, NiCl₂, NiBr₂, NiI₂, etc.), palladium halide (for example, PdF₂, PdCl₂, PdBr₂, PdI₂, etc.), platinum halide (for example, PtF₂, PtCl₂, PtBr₂, PtI₂, etc.), copper halide (for example, CuF, CuCl, CuBr, Cul, etc.), silver halide (for example, AgF, AgCl, AgBr, AgI, etc.), gold halide (for example, AuF, AuCl, AuBr, AuI, etc.), and/or the like.

Examples of the post-transition metal halide are zinc halide (for example, ZnF₂, ZnCl₂, ZnBr₂, ZnI₂, etc.), indium halide (for example, InI₃, etc.), tin halide (for example, SnI₂, etc.), and/or the like.

Examples of the lanthanide metal halide may include YbF, YbF₂, YbF₃, SmF₃, YbCl, YbCl₂, YbCl₃ SmCl₃, YbBr, YbBr₂, YbBr₃, SmBr₃, YbI, YbI₂, YbI₃, SmI₃, and/or the like.

Examples of the metalloid halide are antimony halide (for example, SbCl₅, etc.) and/or the like.

Examples of the metal telluride are alkali metal telluride (for example, Li₂Te, Na₂Te, K₂Te, Rb₂Te, Cs₂Te, etc.), alkaline earth metal telluride (for example, BeTe, MgTe, CaTe, SrTe, BaTe, etc.), transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), post-transition metal telluride (for example, ZnTe, etc.), lanthanide metal telluride (for example, LaTe, CeTe, PrTe, NdTe, PmTe, EuTe, GdTe, TbTe, DyTe, HoTe, ErTe, TmTe, YbTe, LuTe, etc.), and/or the like.

### Emission Layer in Interlayer 130

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers of a red emission layer, a green emission layer, and a blue emission layer, in which the two or more layers contact each other or are separated from each other, to emit white light. In one or more embodiments, the emission layer may include two or more materials of a red light-emitting material, a green light-emitting material, and a blue light-emitting material, in which the two or more materials are mixed with each other in a single layer, to emit white light.

In one or more embodiments, the emission layer may include a host and a dopant (or emitter). In one or more embodiments, the emission layer may further include an auxiliary dopant that promotes energy transfer to a dopant (or emitter), in addition to the host and the dopant (or emitter). When the emission layer includes the dopant (or emitter) and the auxiliary dopant, the dopant (or emitter) and the auxiliary dopant are different from each other.

The transition metal-containing compound represented by Formula 3 in the specification may serve as the dopant (or emitter), or may serve as the auxiliary dopant.

An amount (weight) of the dopant (or emitter) in the emission layer may be in a range of about 0.01 parts by weight to about 15 parts by weight based on 100 parts by weight of the host.

The transition metal-containing compound represented by Formula 3 may be included in the emission layer. An amount (weight) of the transition metal-containing compound in the emission layer may be about 0.01 parts by weight to about 30 parts by weight, about 0.1 parts by weight to about 20 parts by weight, or about 0.1 parts by weight to about 15 parts by weight based on 100 parts by weight of the emission layer.

The thickness of the emission layer may be in a range of about 100 Å to about 1,000 Å, for example, about 200 Å to about 600 Å. When the thickness of the emission layer 120 is within these ranges, excellent or suitable luminescence characteristics may be obtained without a substantial increase in driving voltage.

### Host

The host in the emission layer may include the first compound or the second compound described in the specification, or any combination thereof.

In one or more embodiments, the host may include a compound represented by Formula 301:

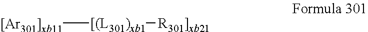

wherein, in Formula 301, Ar₃₀₁ and L₃₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,

xb11 may be 1, 2, or 3,

xb1 may be an integer from 0 to 5,

R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),

xb21 may be an integer from 1 to 5, and

Q₃₀₁ to Q₃₀₃ may each be the same as described in connection with Q₁.

For example, when xb11 in Formula 301 is 2 or more, two or more of Ar₃₀₁ may be linked to each other via a single bond.

In one or more embodiments, the host may include a compound represented by Formula 301-1, a compound represented by Formula 301-2, or any combination thereof:

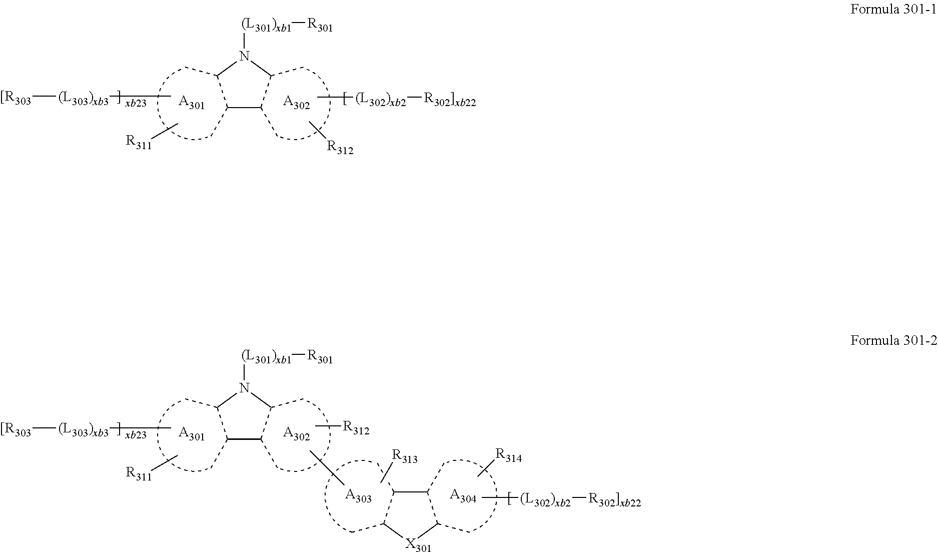

wherein, in Formulae 301-1 and 301-2, ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

X₃₀₁ may be O, S, N-[(L₃₀₄)_{xb4}-R₃₀₄], C(R₃₀₄)(R₃₀₅), or Si(R₃₀₄)(R₃₀₅),

xb22 and xb23 may each independently be 0, 1, or 2,

L₃₀₁, xb1, and R₃₀₁ may each be the same as described herein,

L₃₀₂ to L₃₀₄ may each independently be the same as described in connection with L₃₀₁,

xb2 to xb4 may each independently be the same as described in connection with xb1, and

R₃₀₂ to R₃₀₅ and R₃₁₁ to R₃₁₄ may each be the same as described in connection with R₃₀₁.

In one or more embodiments, the host may include an alkali earth metal complex, a post-transition metal complex, or any combination thereof. In one or more embodiments, the host may include a Be complex (for example, Compound H55), an Mg complex, a Zn complex, or any combination thereof.

In one or more embodiments, the host may include: one of selected from among Compounds H1 to H130; 9,10-di(2-naphthyl)anthracene (ADN); 2-methyl-9,10-bis(naphthalen-2-yl)anthracene (MADN); 9,10-di(2-naphthyl)-2-t-butyl-anthracene (TBADN); 4,4′-bis(N-carbazolyl)-1,1′-biphenyl (CBP); 1,3-di-9-carbazolylbenzene (mCP); 1,3,5-tri(carbazol-9-yl)benzene (TCP); and/or any combination thereof:

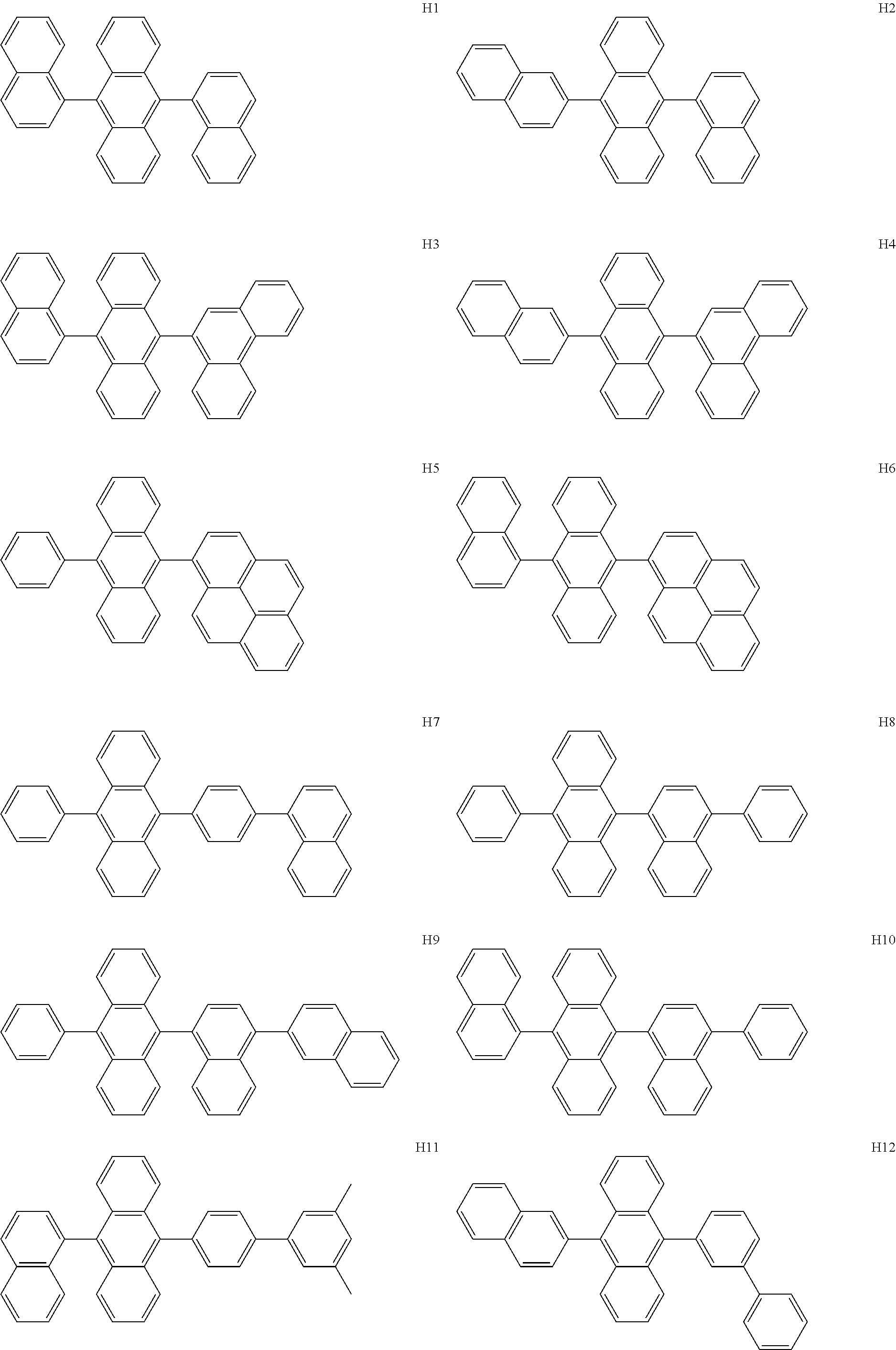

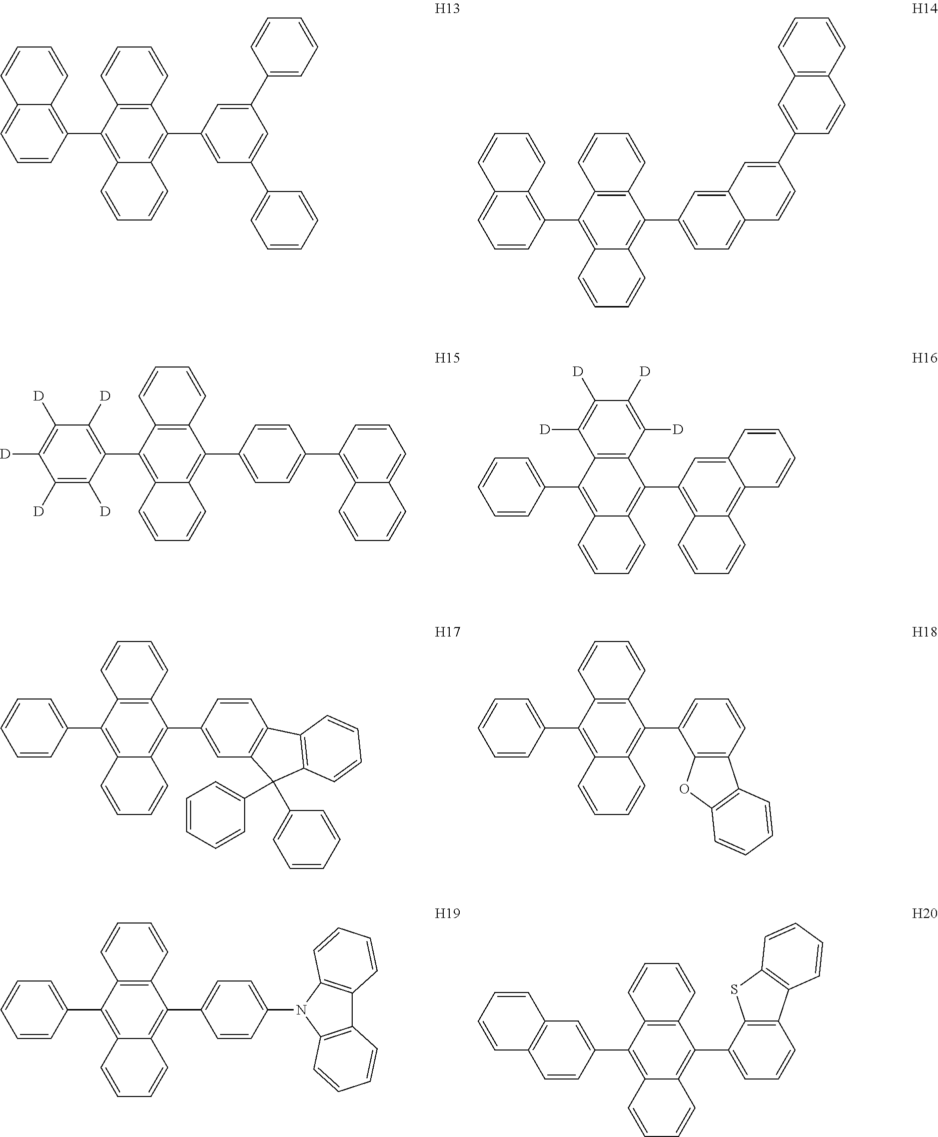

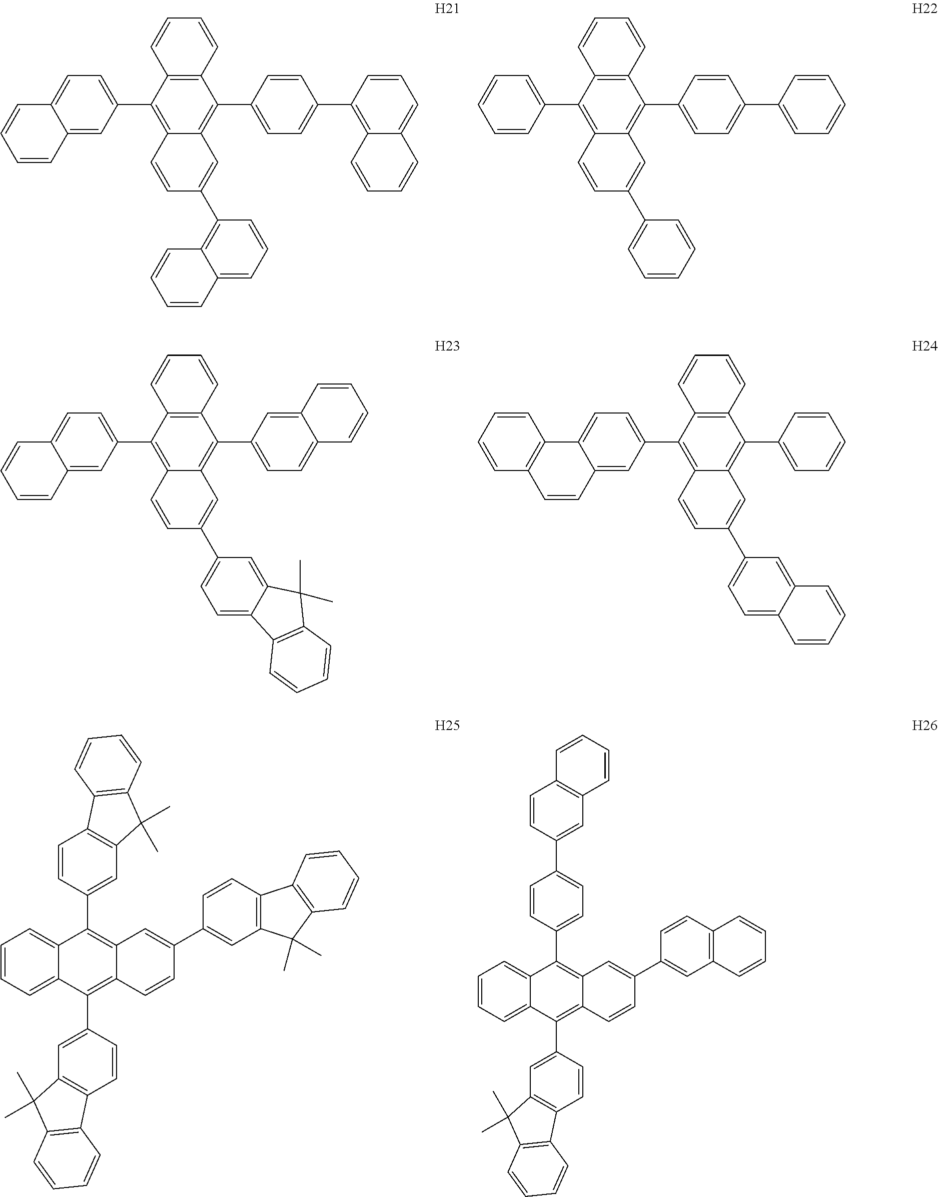

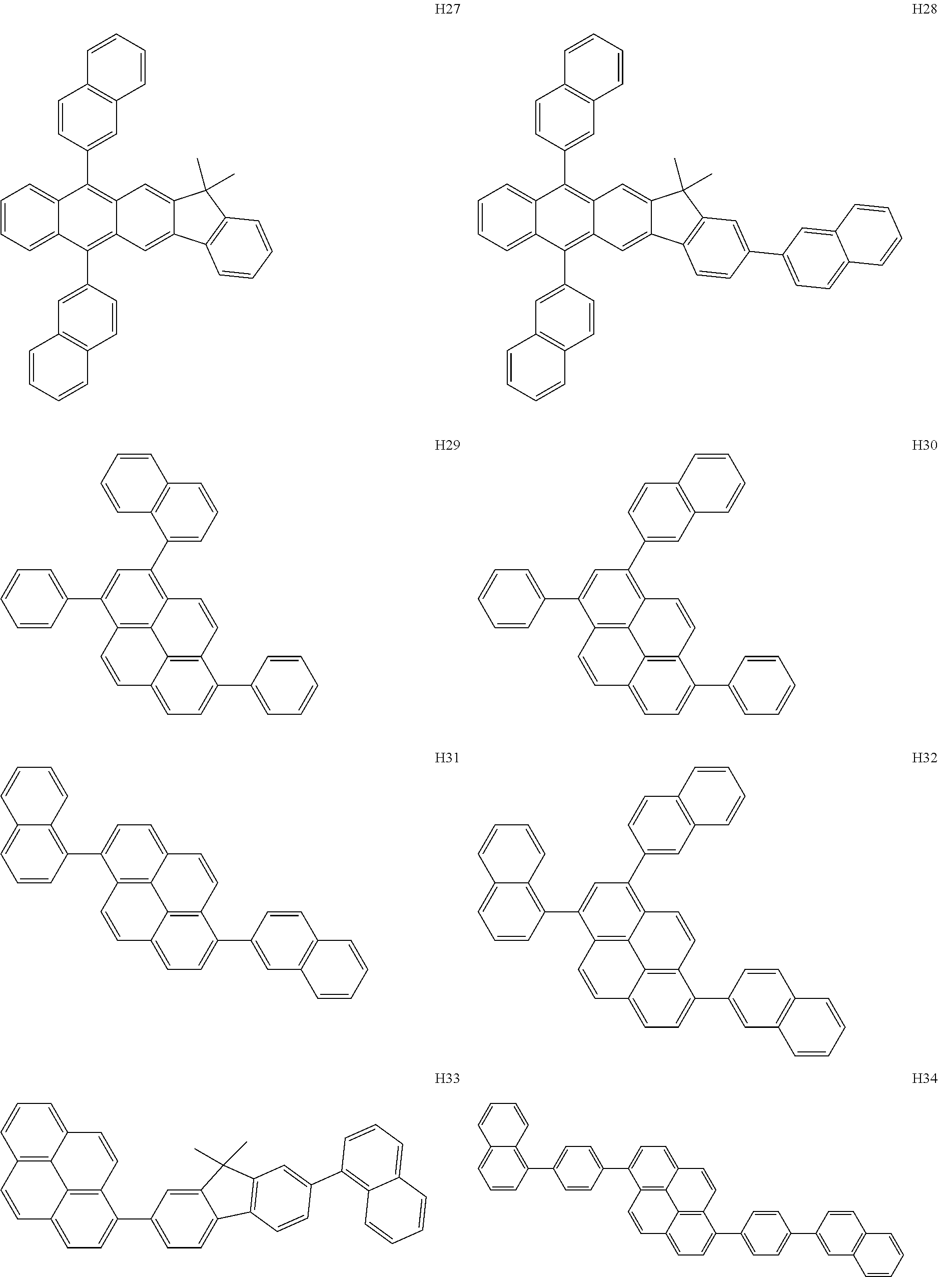

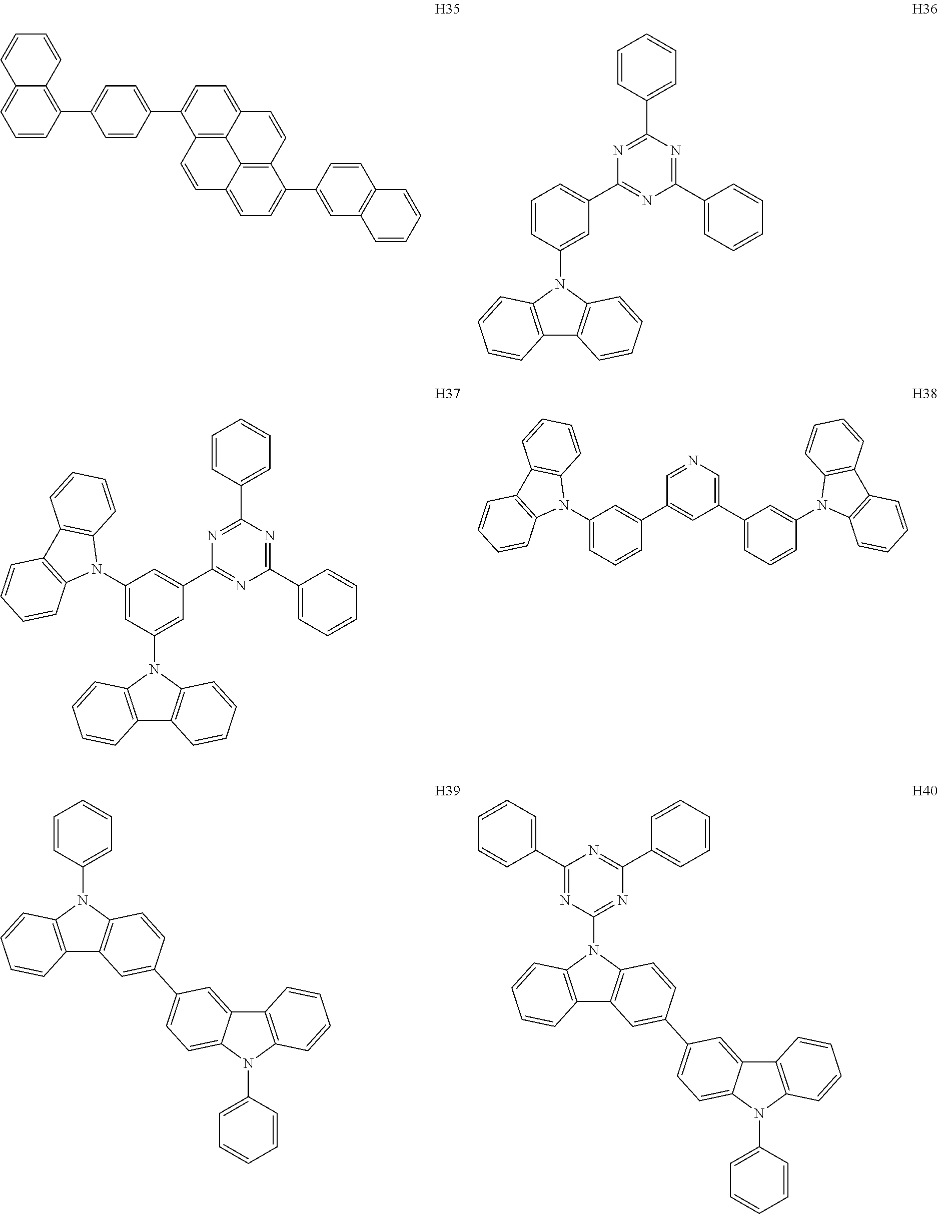

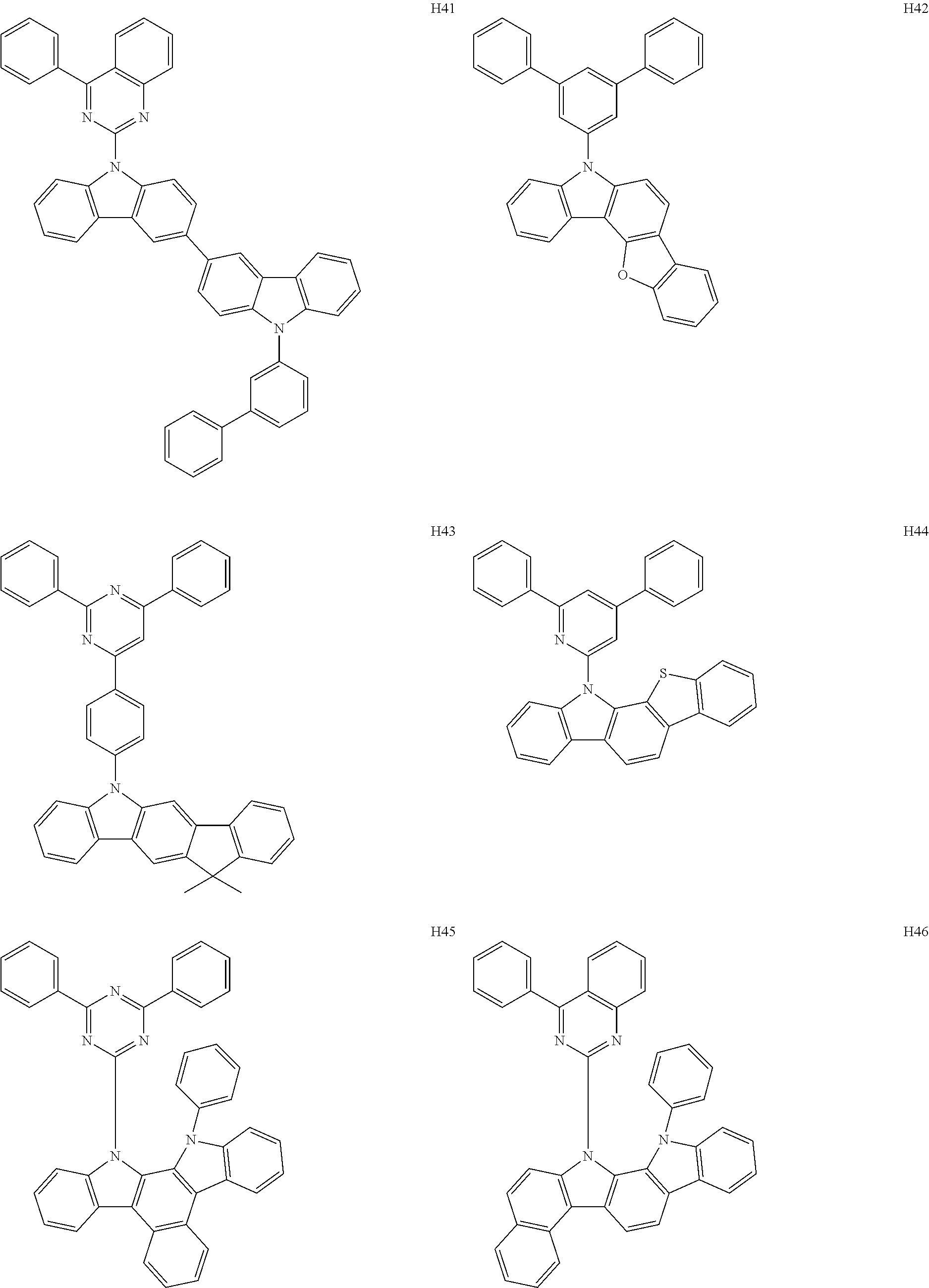 [SMILES: c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1 ; CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-n3c4ccc5ccccc5c4c4ccc5c6ccccc6n(-c6ccccc6)c5c43)nc3ccccc23)cc1]

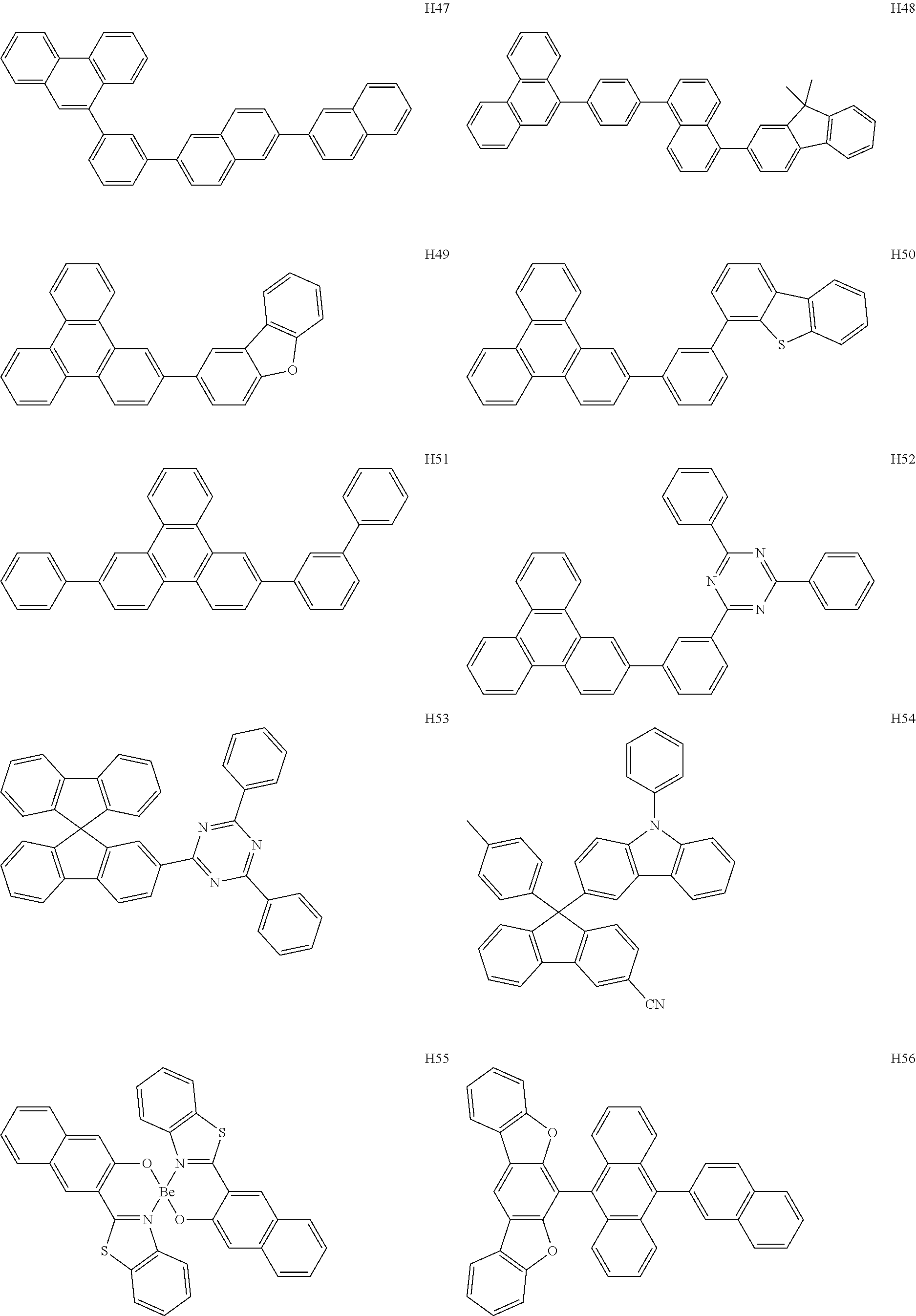

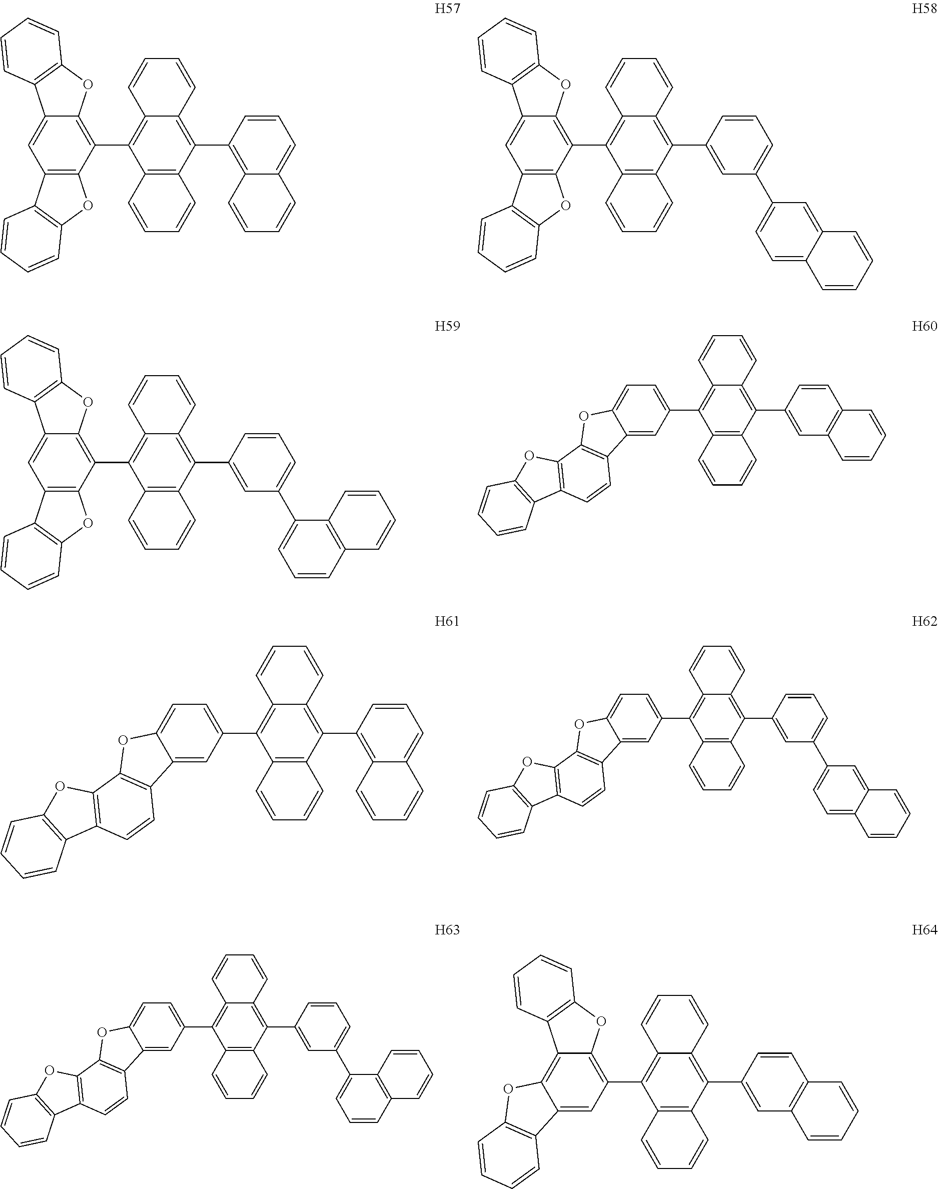

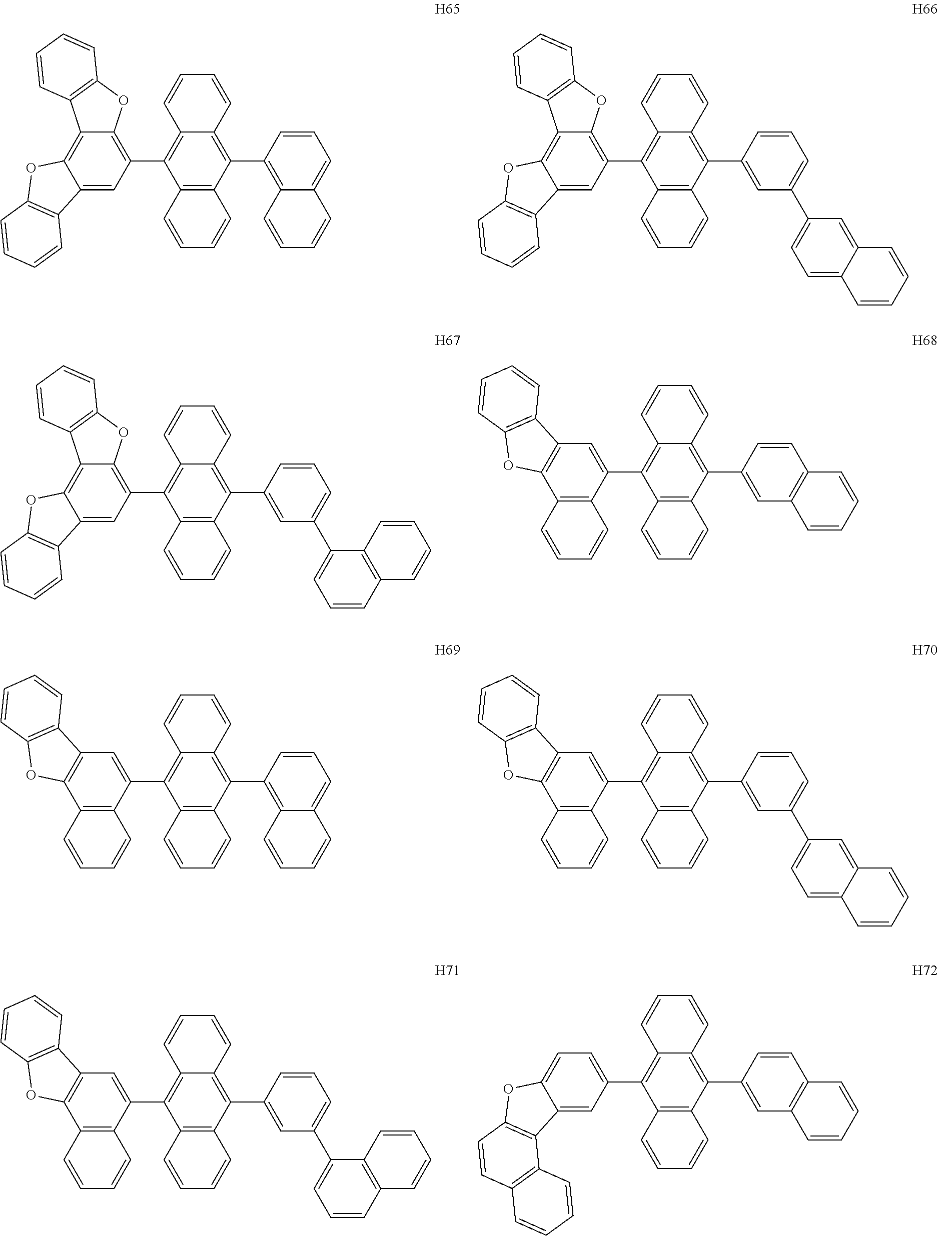

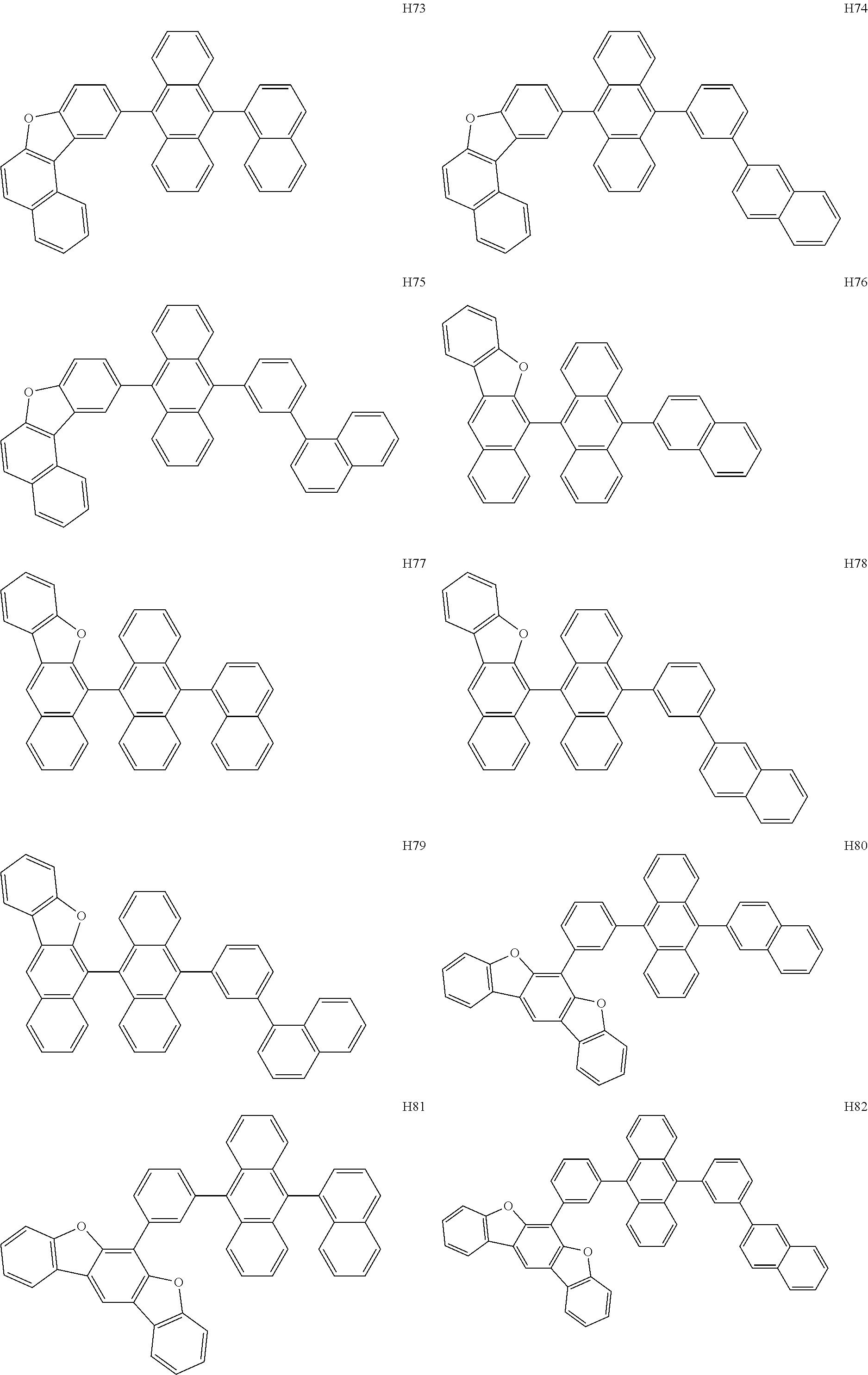

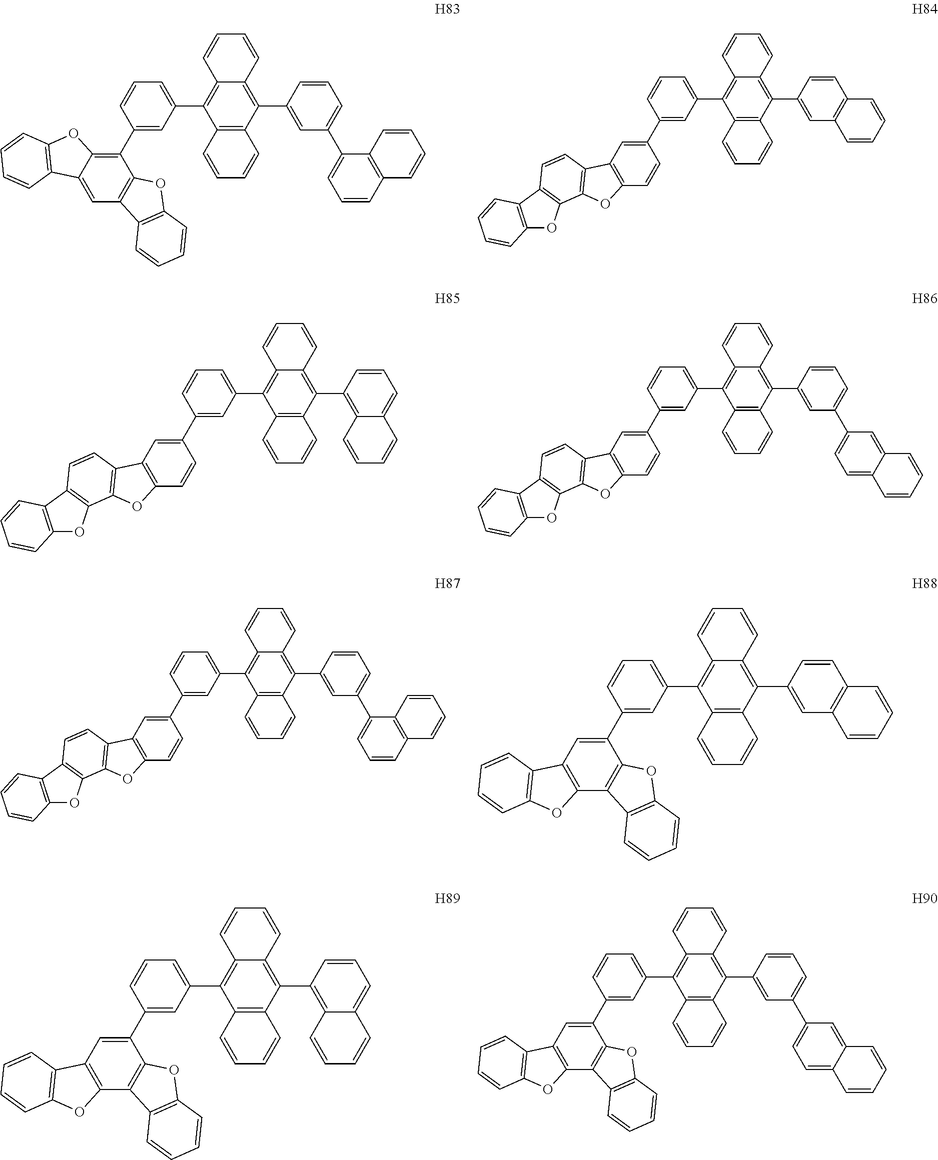

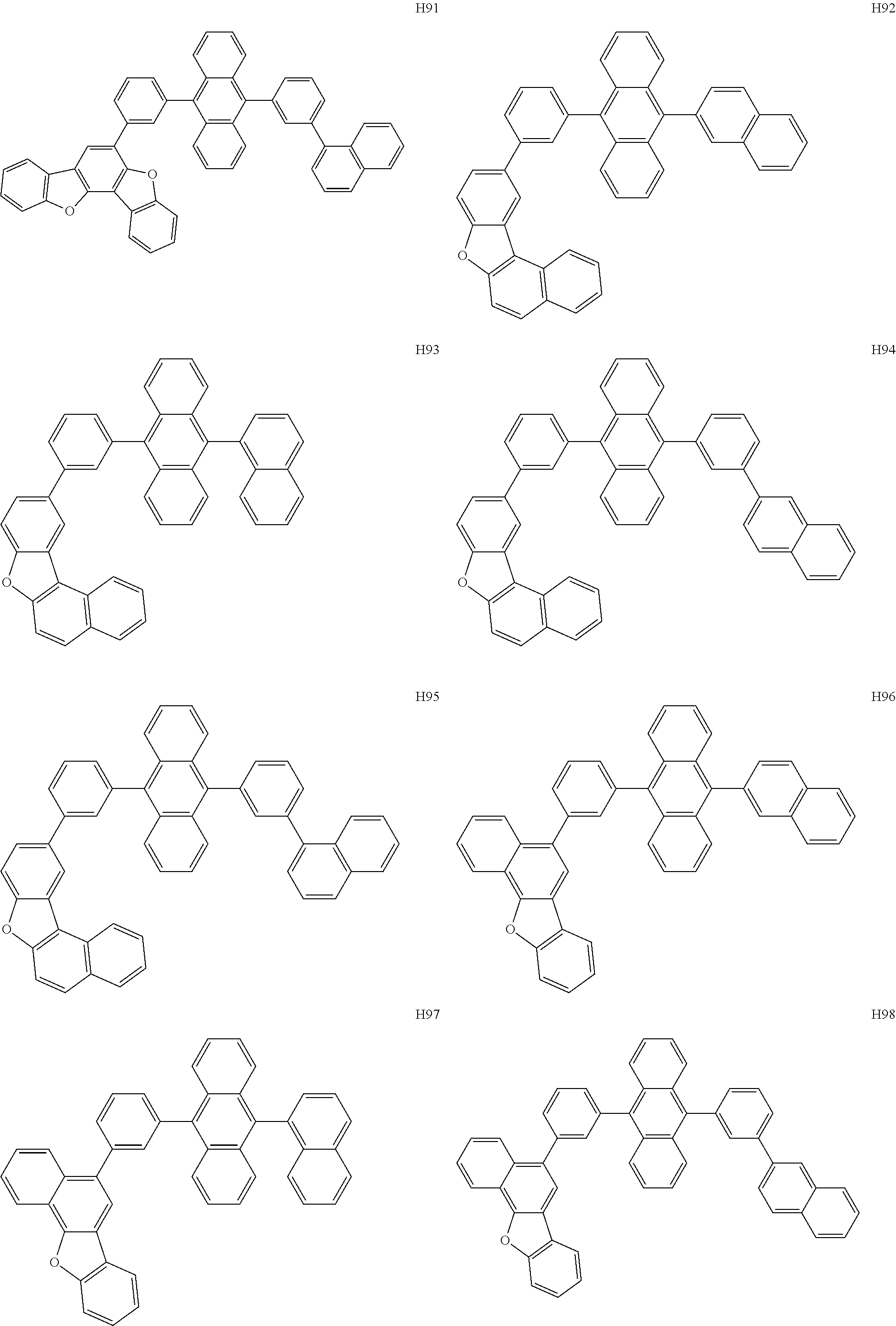

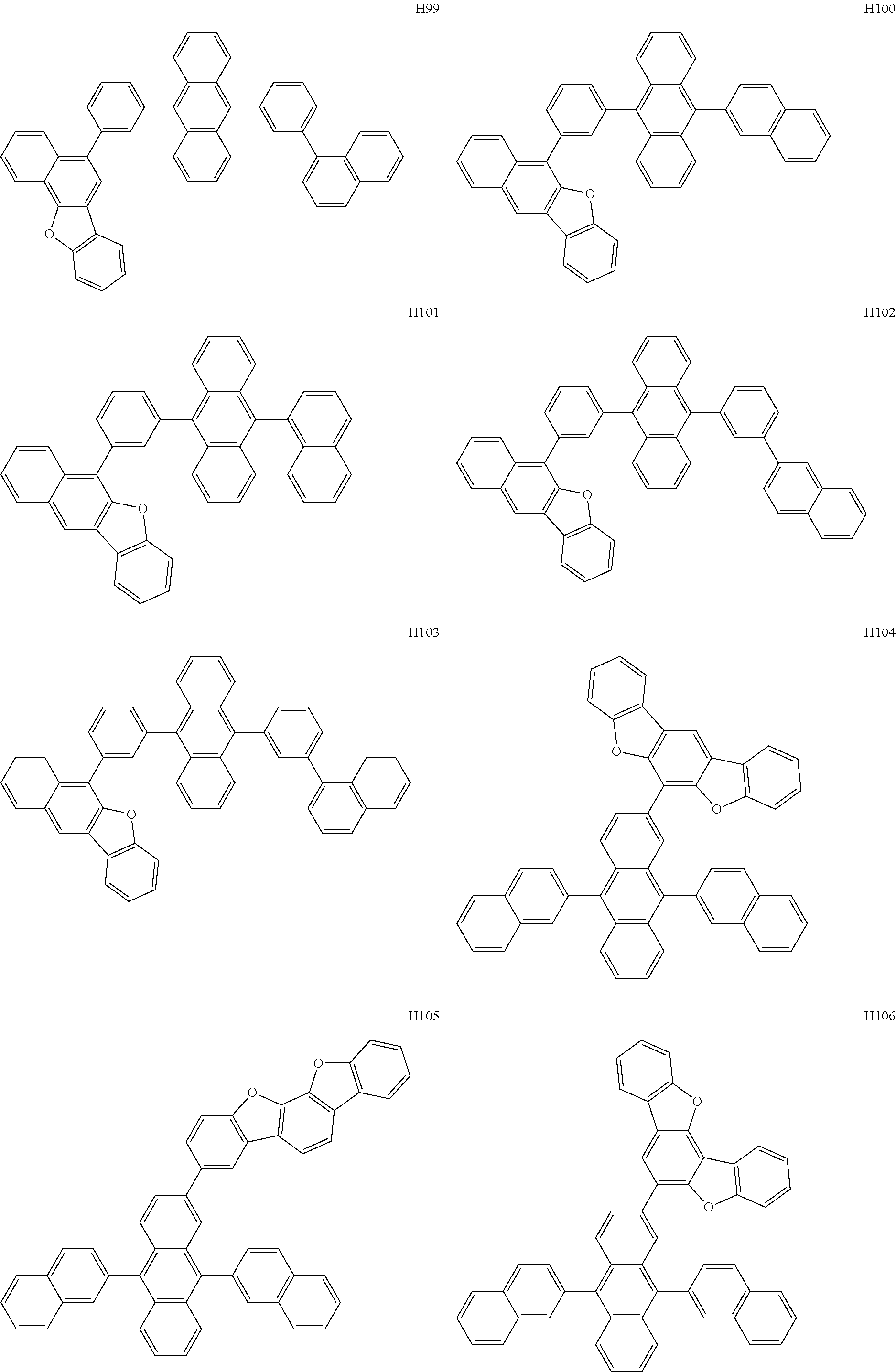

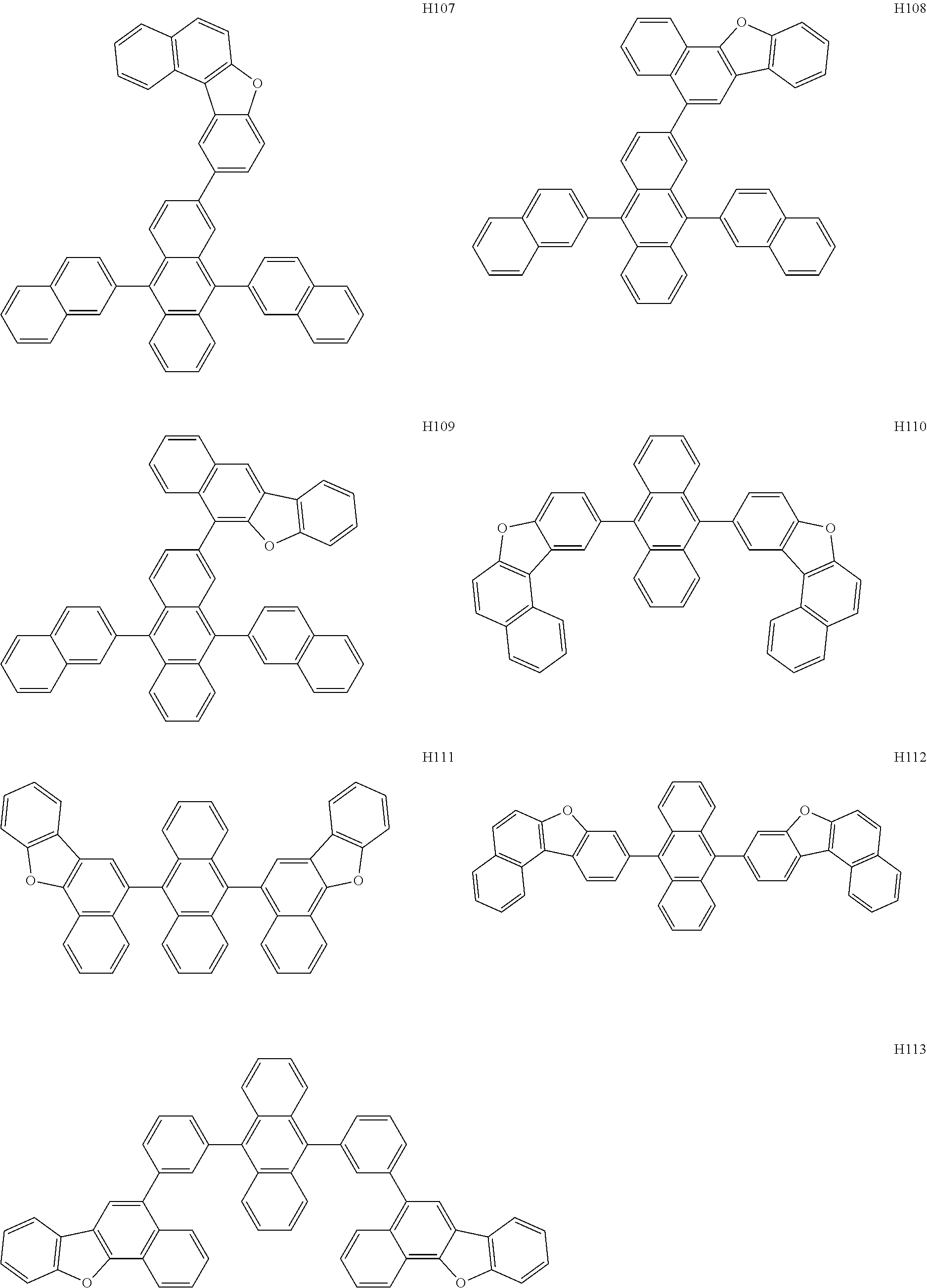

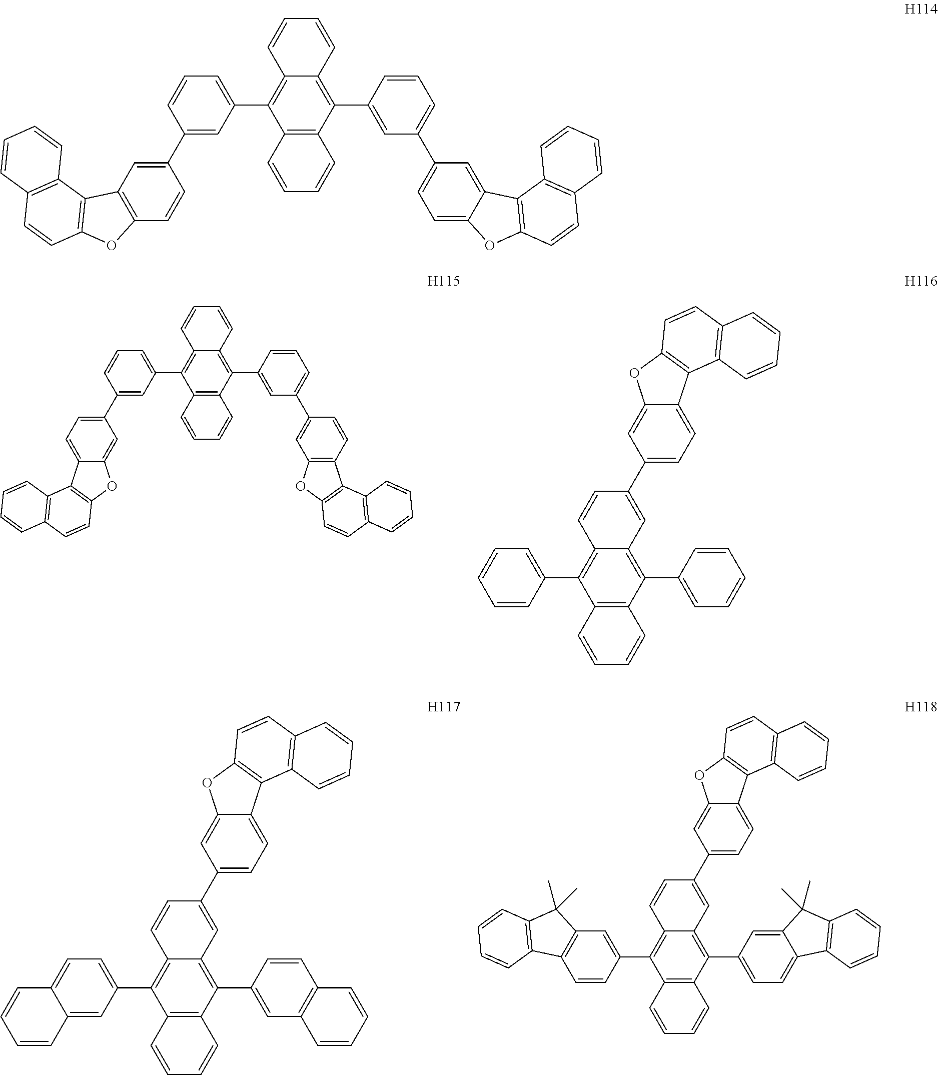 [SMILES: c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

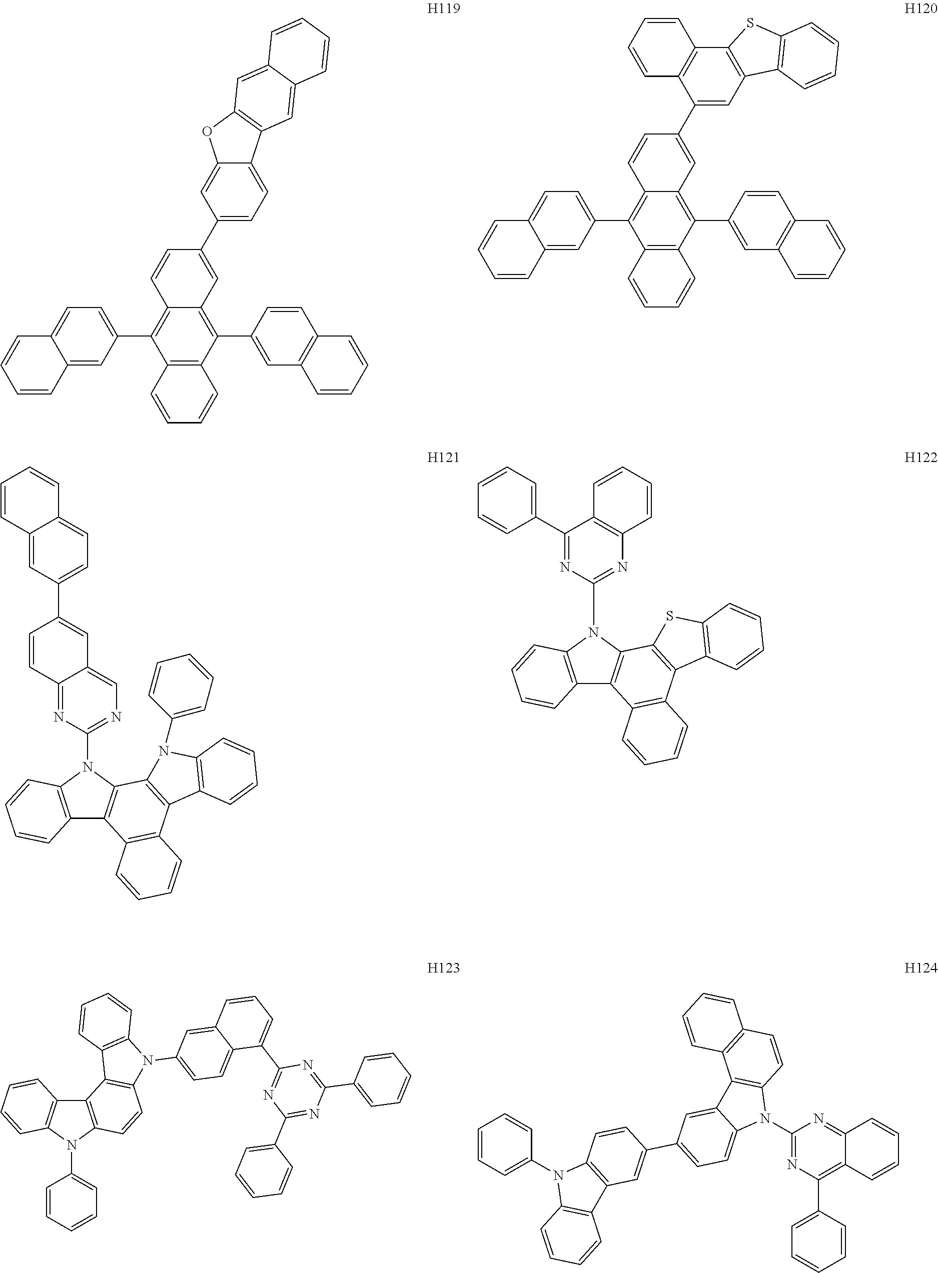

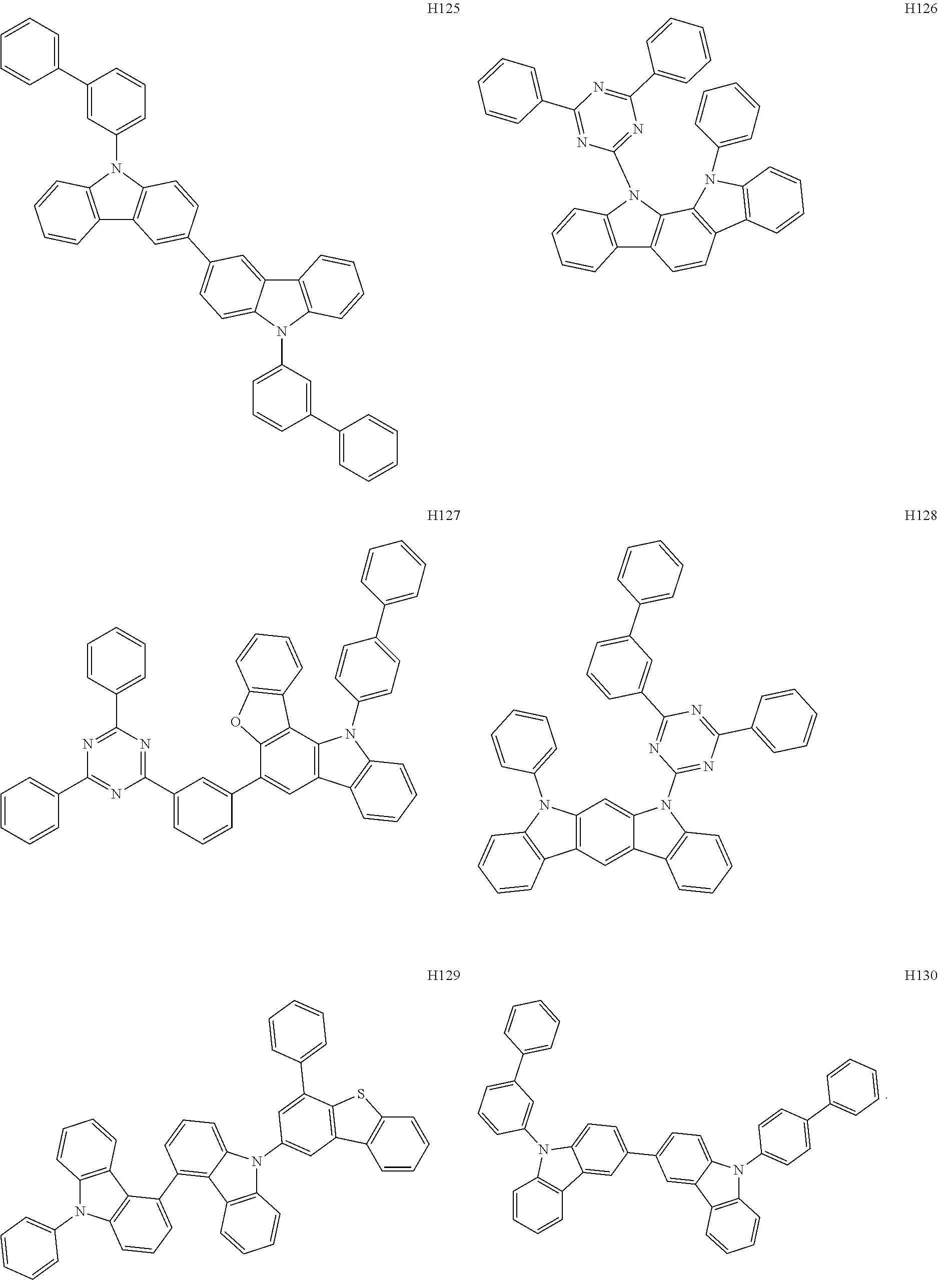

In one or more embodiments, the host may include a silicon-containing compound, a phosphine oxide-containing compound, or any combination thereof.

The host may have one or more suitable modifications. For example, the host may include only one kind of compound, or may include two or more kinds of different compounds.

### Phosphorescent Dopant

The emission layer may include, as a phosphorescent dopant, the transition metal-containing compound represented by Formula 3 as described in the specification.

Or the emission layer may include the transition metal-containing compound represented by Formula 3 as described in the specification, and when the transition metal-containing compound represented by Formula 3 as described in the specification serves as an auxiliary dopant, the emission layer may include a phosphorescent dopant.

The phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include a transition metal-containing compound represented by Formula 401 and/or Formula 402:

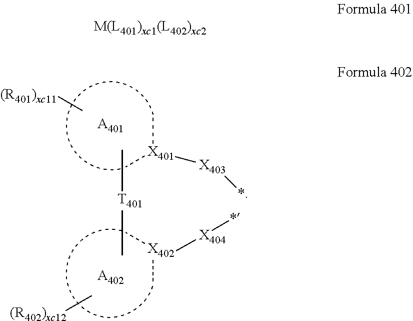

In Formulae 401 and 402, M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),

L₄₀₁ may be a ligand represented by Formula 402, and xc1 is 1, 2, or 3, wherein, when xc1 is 2 or more, two or more of L₄₀₁ may be identical to or different from each other,

L₄₀₂ may be an organic ligand, and xc2 may be 0, 1, 2, 3, or 4, wherein, when xc2 is 2 or more, two or more of L₄₀₂ may be identical to or different from each other,

X₄₀₁ and X₄₀₂ may each independently be N or C,

ring A₄₀₁ and ring A₄₀₂ may each independently be a C₃-C₆₀ carbocyclic group or a C₁-C₆₀ heterocyclic group,

T₄₀₁ may be a single bond, *—O—*′, *—S—*′, *—C(═O)—*, *—N(Q₄₁₁)*′, *—C(Q₄₁₁)(Q₄₁₂)-*′, *—C(Q₄₁₁)═C(Q₄₁₂)-*′, *—C(Q₄₁₁)=*′, or *═C(Q₄₁₁)=*′,

X₄₀₃ and X₄₀₄ may each independently be a chemical bond (for example, a covalent bond or a coordination bond), O, S, N(Q₄₁₃), B(Q₄₁₃), P(Q₄₁₃), C(Q₄₁₃)(Q₄₁₄), or Si(Q₄₁₃)(Q₄₁₄),

Q₄₁₁ to Q₄₁₄ may each be the same as described in connection with Q₁,

R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),

Q₄₀₁ to Q₄₀₃ may each be the same as described in connection with Q₁,

xc11 and xc12 may each independently be an integer from 0 to 10, and

* and *′ in Formula 402 each indicate a binding site to M in Formula 401.

For example, in Formula 402, i) X₄₀₁ may be nitrogen and X₄₀₂ may be carbon, or ii) each of X₄₀₁ and X₄₀₂ may be nitrogen.

In one or more embodiments, when xc1 in Formula 402 is 2 or more, two ring A₄₀₁ (s) in two or more of L₄₀₁(s) may be optionally linked to each other via T₄₀₂, which is a linking group, or two ring A₄₀₂(s) may be optionally linked to each other via T₄₀₃, which is a linking group (see Compounds PD1 to PD4 and PD7). T₄₀₂ and T₄₀₃ may each independently be as defined in T₄₀₁.

In Formula 401, L₄₀₂ may be an organic ligand. For example, L₄₀₂ may include a halogen group, a diketone group (for example, an acetylacetonate group), a carboxylic acid group (for example, a picolinate group), —C(═O), an isonitrile group, a —CN group, a phosphorus group (for example, a phosphine group, a phosphite group, etc.), or any combination thereof.

The phosphorescent dopant may include, for example, one of selected from among compounds PD1 to PD25, and/or any combination thereof:

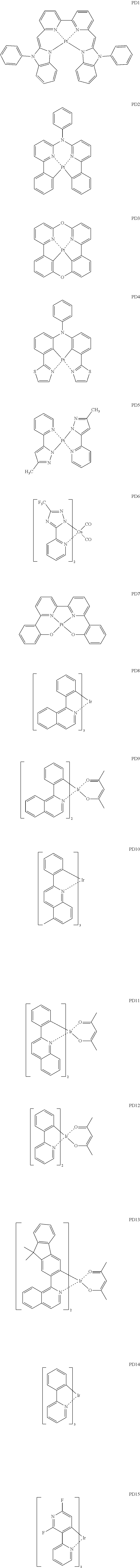

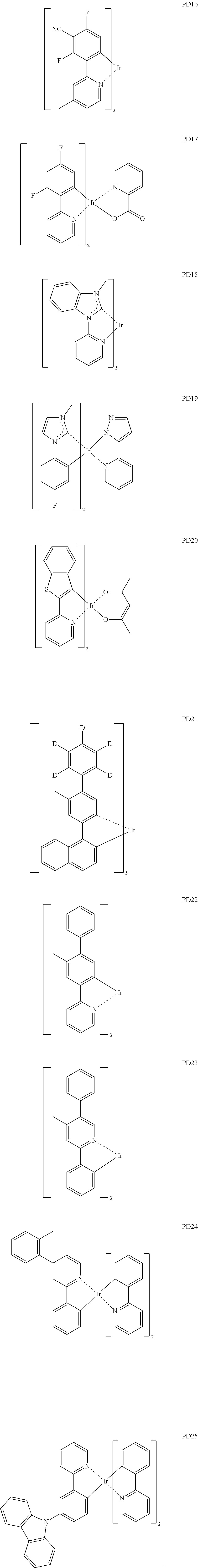

### Fluorescent Dopant

The emission layer may include transition metal-containing compound represented by Formula 3 as described in the specification, and when the transition metal-containing compound represented by Formula 3 as described in the specification serves as an auxiliary dopant, the emission layer may further include a fluorescent dopant.

Or the emission layer may include the transition metal-containing compound represented by Formula 3 as described in the specification, and when the transition metal-containing compound represented by Formula 3 as described in the specification serves as a phosphorescent dopant, the emission layer may further include an auxiliary dopant.

The fluorescent dopant and the auxiliary dopant may each independently include an arylamine compound, a styrylamine compound, a boron-containing compound, or any combination thereof.

In one or more embodiments, the fluorescent dopant and the auxiliary dopant may each independently include a compound represented by Formula 501:

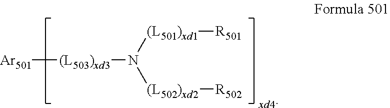

In Formula 501, Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xd1 to xd3 may each independently be 0, 1, 2, or 3, and

xd4 may be 1, 2, 3, 4, 5, or 6.

In one or more embodiments, Ar₅₀₁ in Formula 501 may be a condensed cyclic group (for example, an anthracene group, a chrysene group, a pyrene group, etc.) in which three or more monocyclic groups are condensed together.

In one or more embodiments, xd4 in Formula 501 may be 2.

In one or more embodiments, the fluorescent dopant and the auxiliary dopant may each include one of selected from among Compounds FD1 to FD36, DPVBi, DPAVBi, and/or any combination thereof:

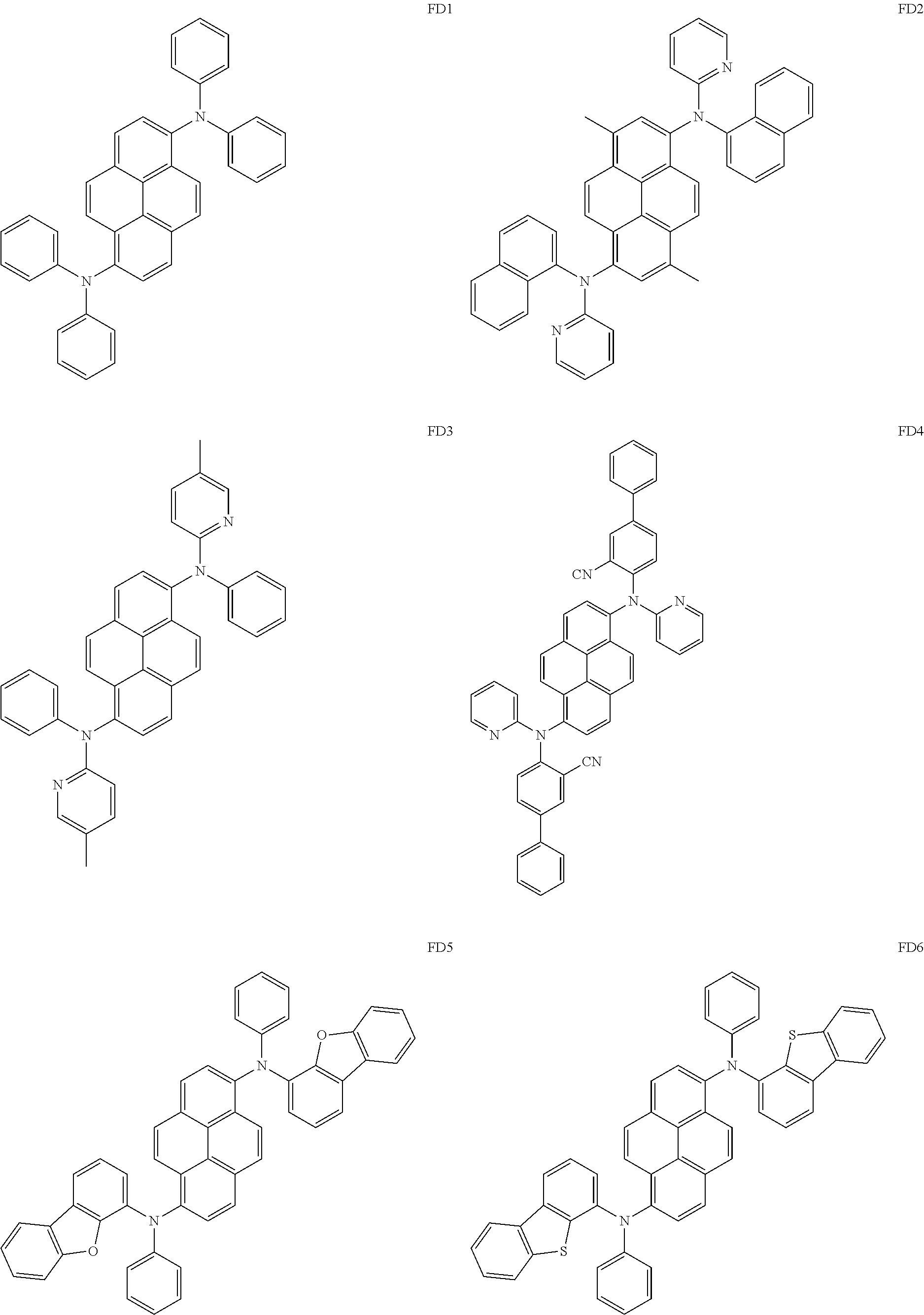

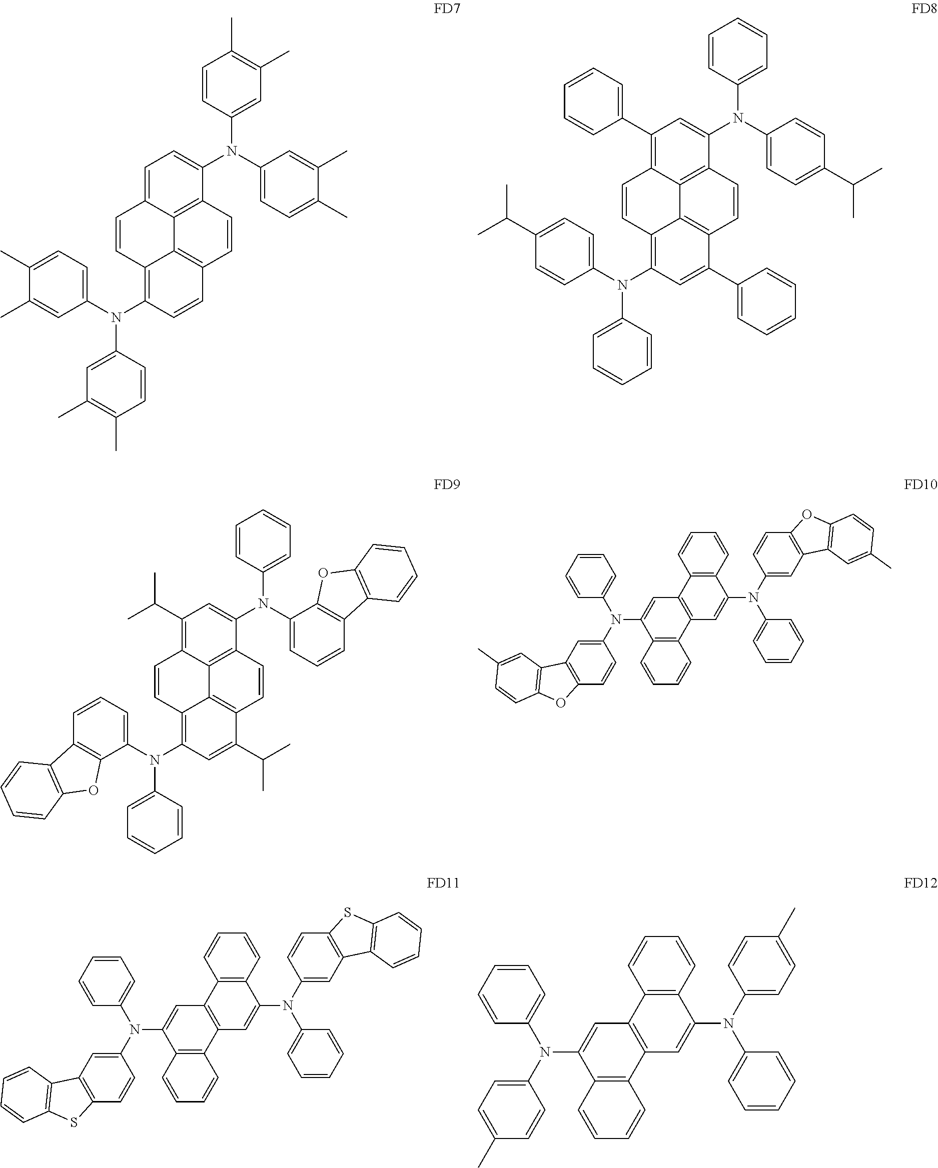

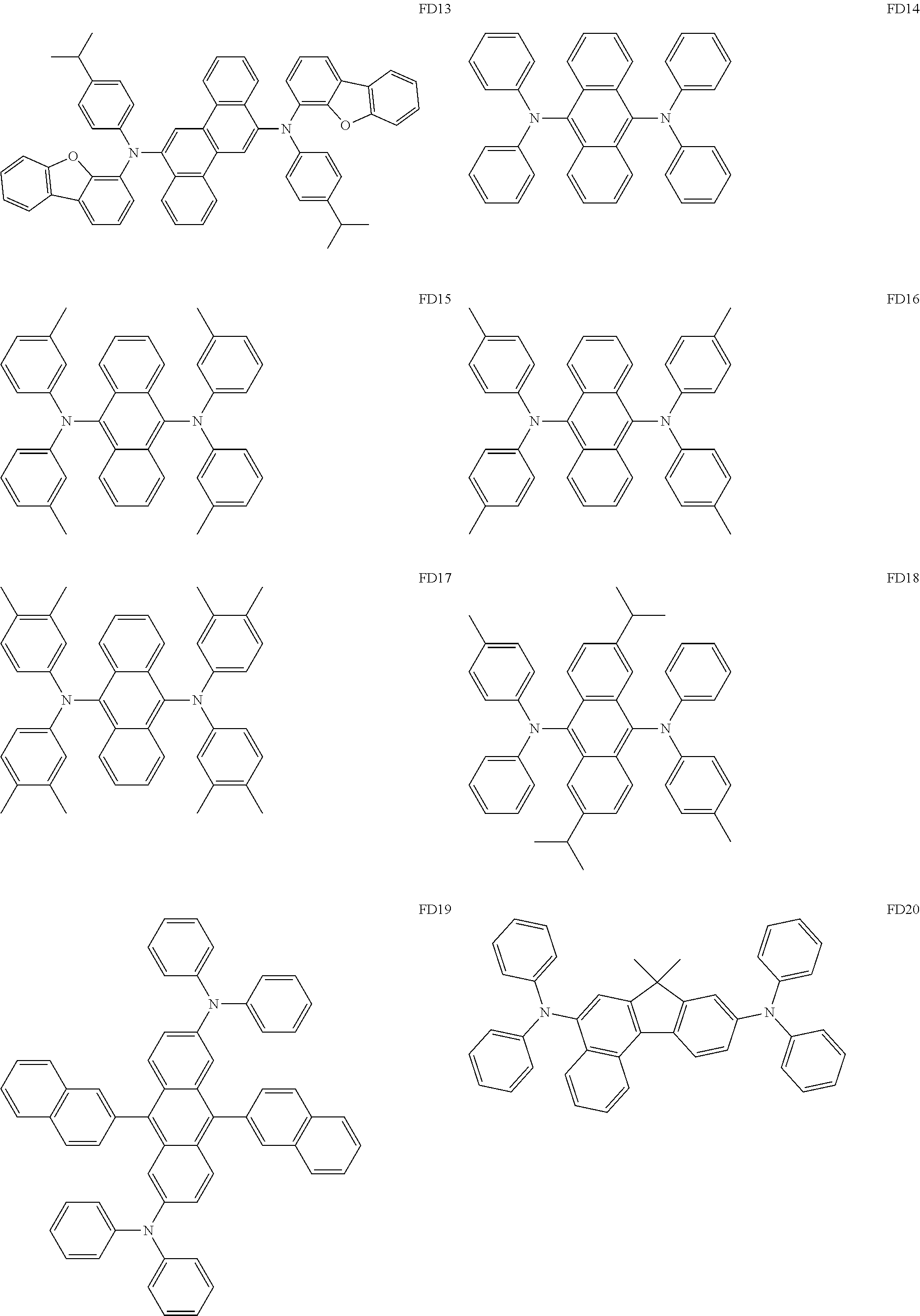

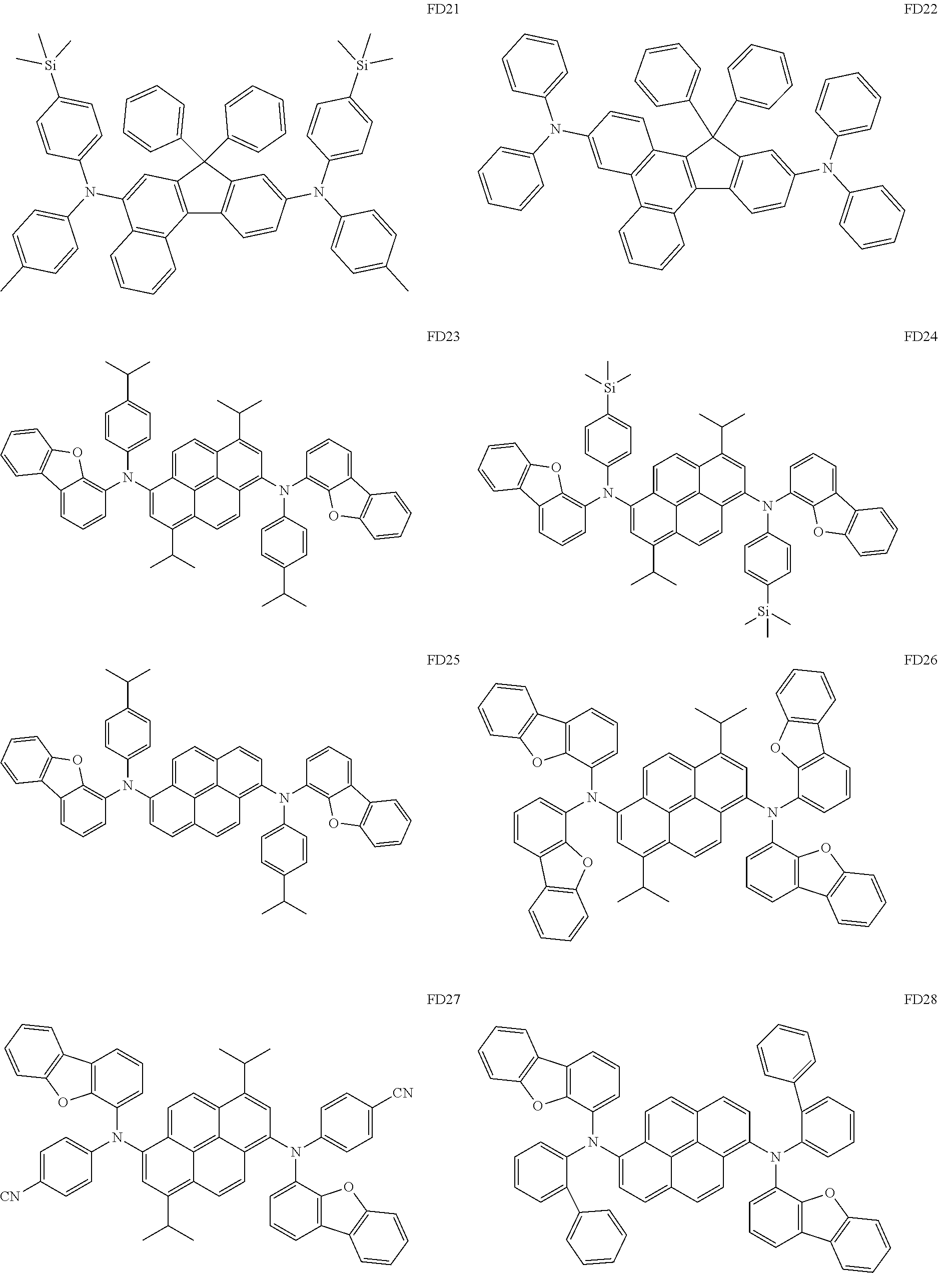

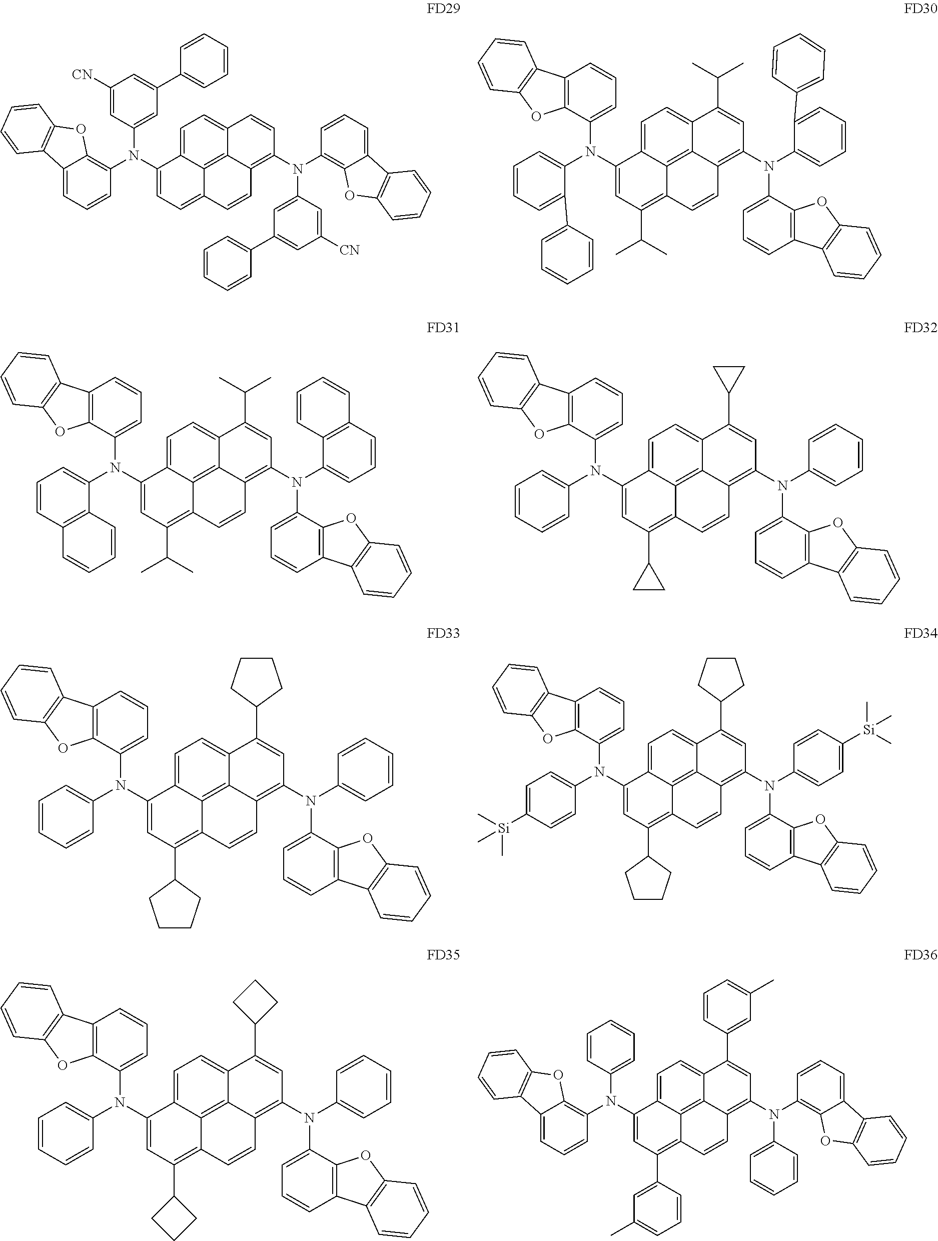

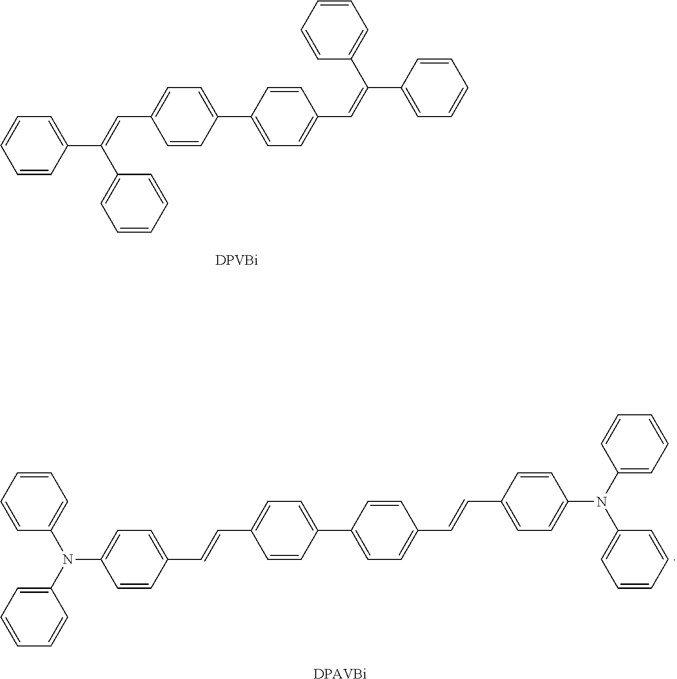

In one or more embodiments, the fluorescent dopant and the auxiliary dopant may each independently include a delayed fluorescence compound PP3C, represented by Formula 502 or 503 as described herein.

### Electron Transport Region in Interlayer 130

The electron transport region may have: i) a single-layer structure including (e.g., consisting of) a single layer including (e.g., consisting of) a single material, ii) a single-layer structure including (e.g., consisting of) a single layer including (e.g., consisting of) multiple different materials, or iii) a multi-layer structure including multiple layers including different materials.

The electron transport region may include a buffer layer, a hole blocking layer, an electron control layer, an electron transport layer, an electron injection layer, or any combination thereof.

For example, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein constituent layers of each structure are sequentially stacked from the emission layer.

The electron transport region 140 (for example, the buffer layer, the hole blocking layer, the electron control layer, or the electron transport layer in the electron transport region) may include a metal-free compound including at least one rr electron-deficient nitrogen-containing C₁-C₆₀ heterocyclic group.

For example, the electron transport region may include a compound represented by Formula 601:

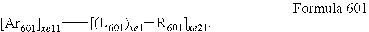

In Formula 601, Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xe11 may be 1, 2, or 3,

xe1 may be 0, 1, 2, 3, 4, or 5,

R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),

Q₆₀₁ to Q₆₀₃ may each be the same as described in connection with Q₁,

xe21 may be 1,2, 3, 4, or 5, and

at least one of selected from Ar₆₀₁, L₆₀₁, and R₆₀₁ may each independently be a π electron-deficient nitrogen-containing C₁-C₆₀ heterocyclic group unsubstituted or substituted with at least one R₁₀ₐ.

In one or more embodiments, when xe11 in Formula 601 is 2 or more, two or more of Ar₆₀₁ may be linked to each other via a single bond.

In one or more embodiments, Ar₆₀₁ in Formula 601 may be a substituted or unsubstituted anthracene group.

In one or more embodiments, the electron transport region may include a compound represented by Formula 601-1:

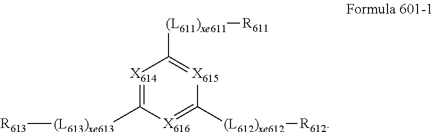

In Formula 601-1, X₆₁₄ may be N or C(R₆₁₄), X₆₁₅ may be N or C(R₆₁₅), X₆₁₆ may be N or C(R₆₁₆), and at least one of selected from among X₆₁₄ to X₆₁₆ may be N,

L₆₁₁ to L₆₁₃ may each be the same as described in connection with L₆₀₁,

xe611 to xe613 may each be the same as described in connection with xe1,

R₆₁₁ to R₆₁₃ may each be the same as described in connection with R₆₀₁, and

R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region may include: one of selected from among Compounds ET1 to ET46; 2,9-dimethyl-4,7-diphenyl-1,10-phenanthroline (BOP); 4,7-diphenyl-1,10-phenanthroline (Bphen); Alq₃; BAlq; TAZ; NTAZ; and/or any combination thereof:

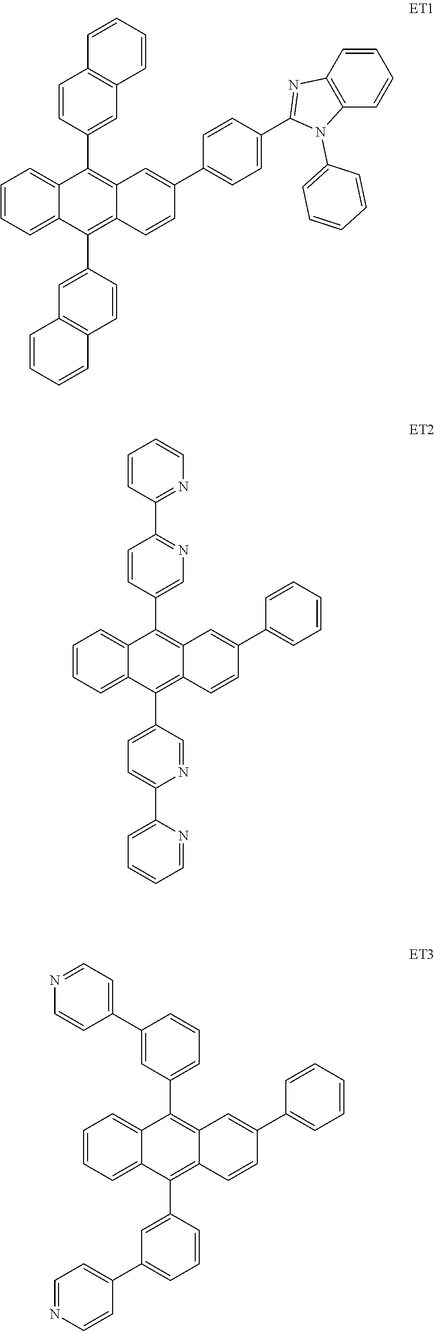 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

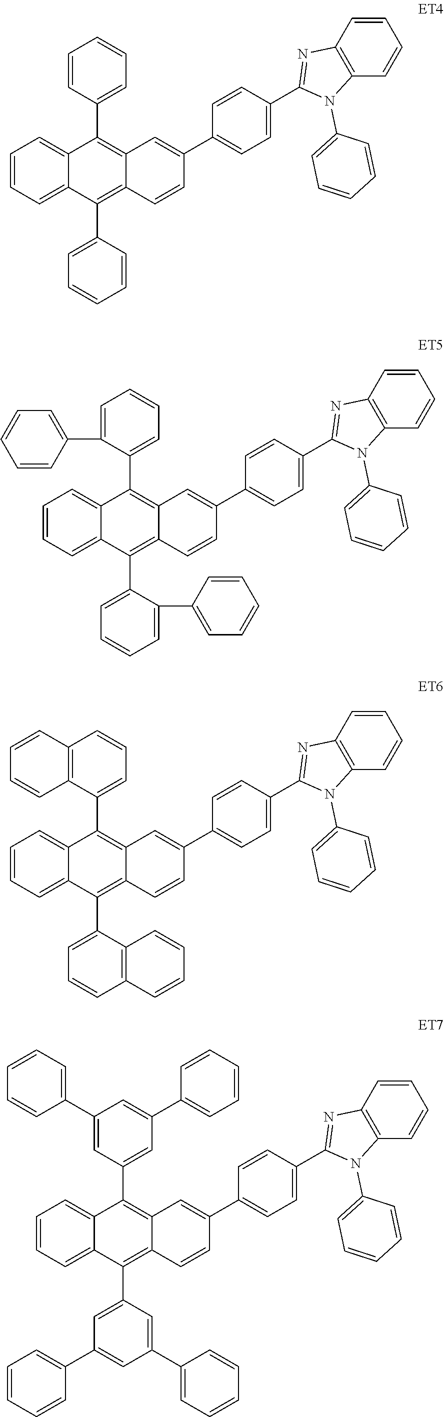 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

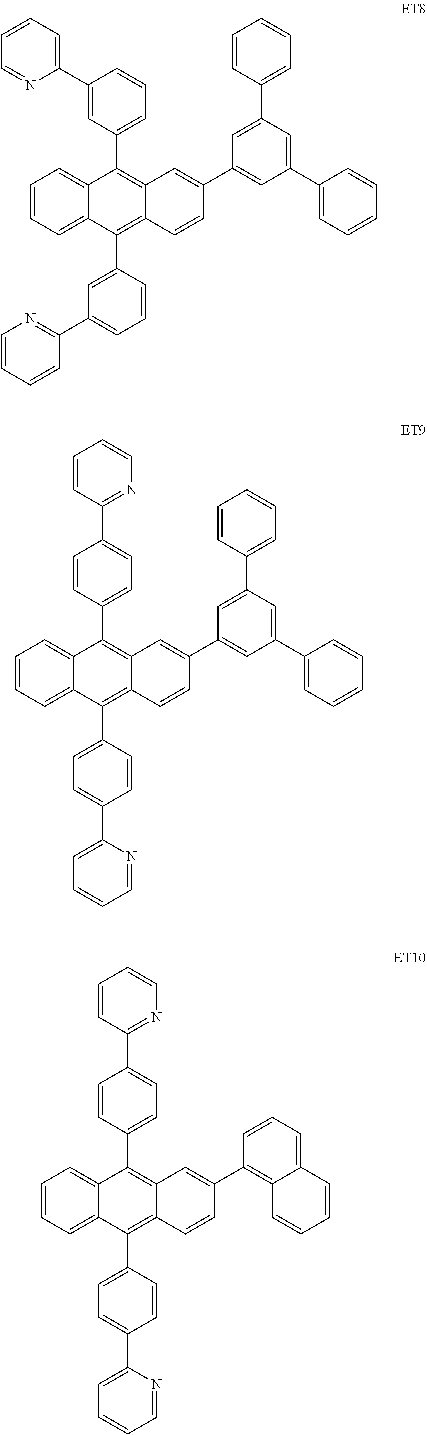 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

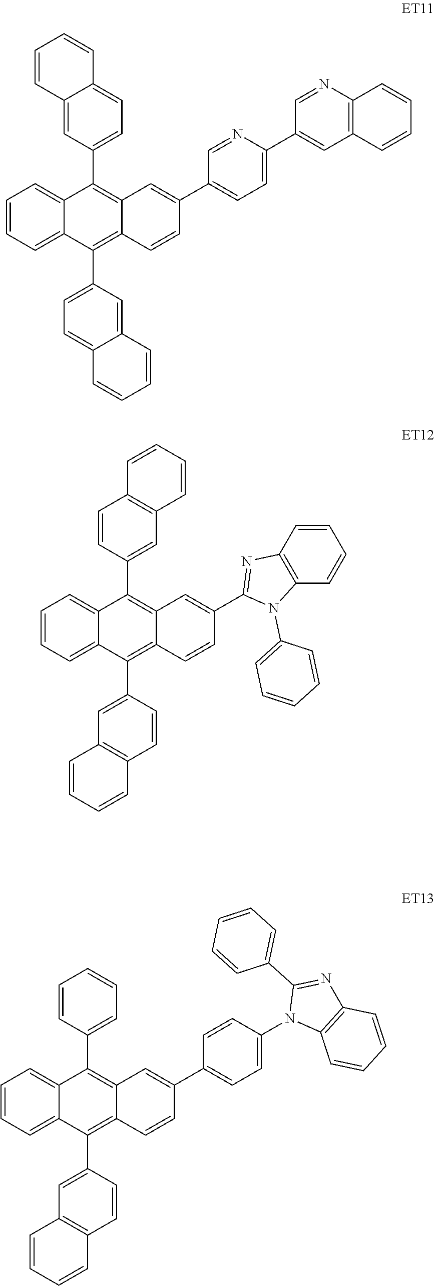 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

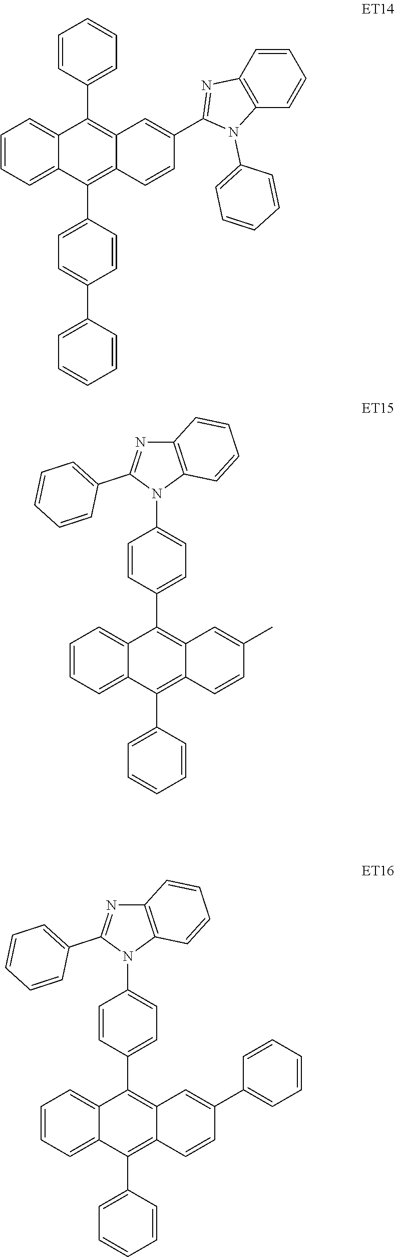 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

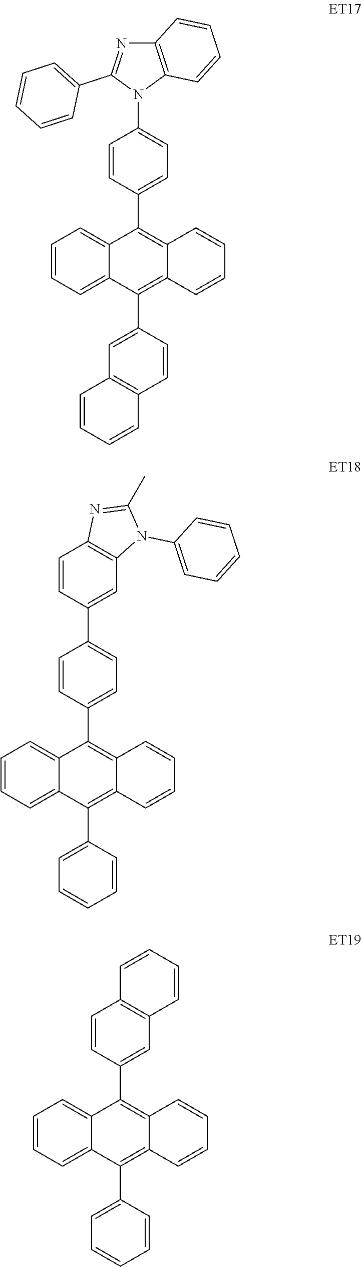

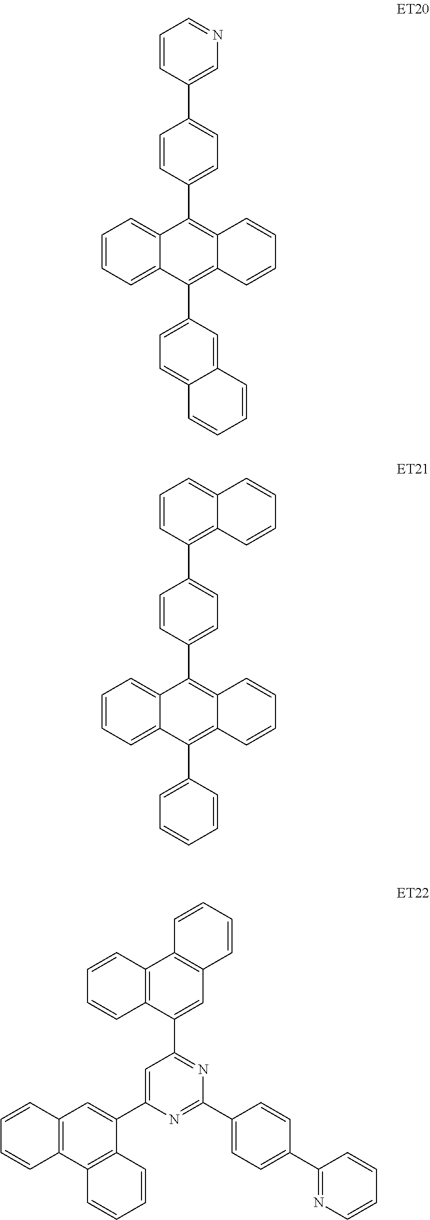

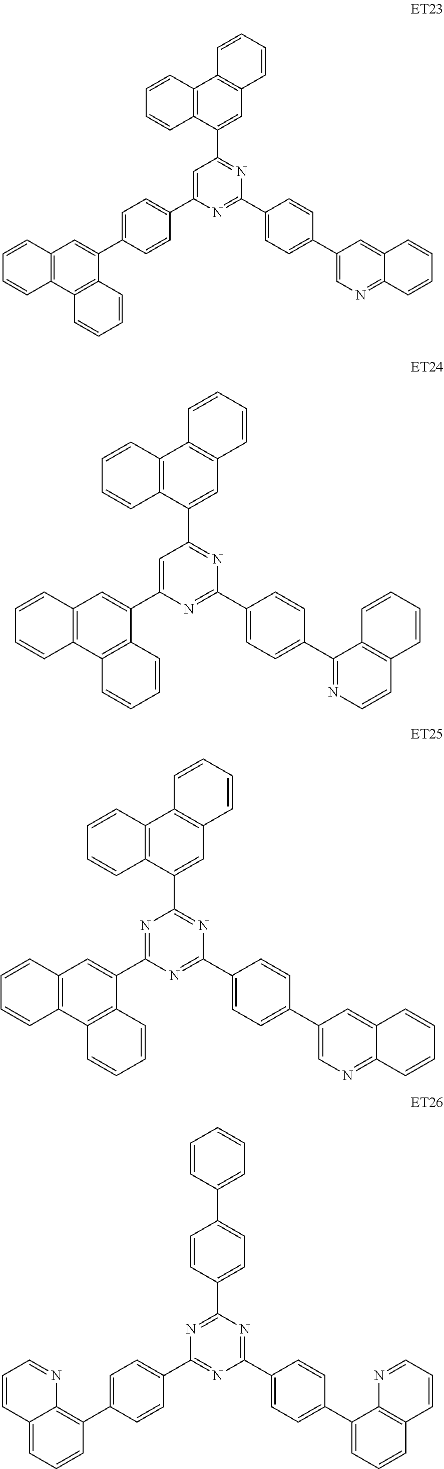 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1]

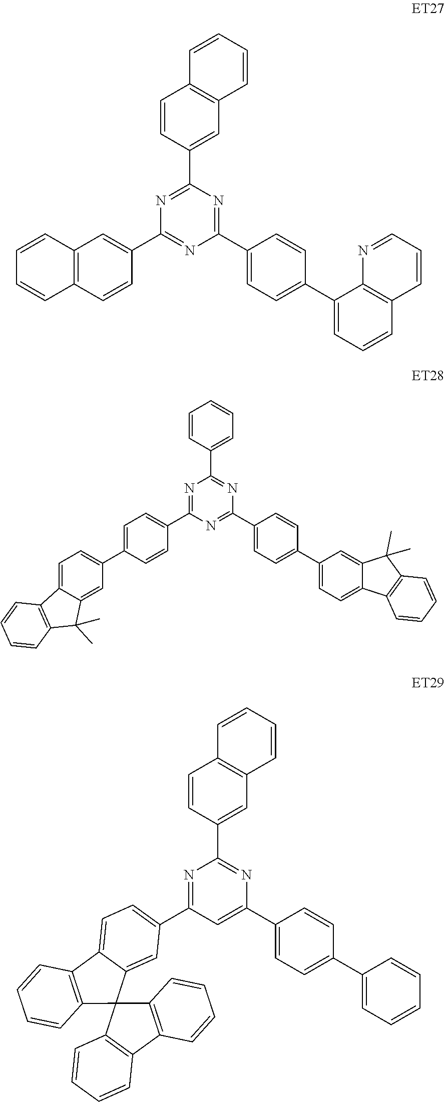 [SMILES: c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1]

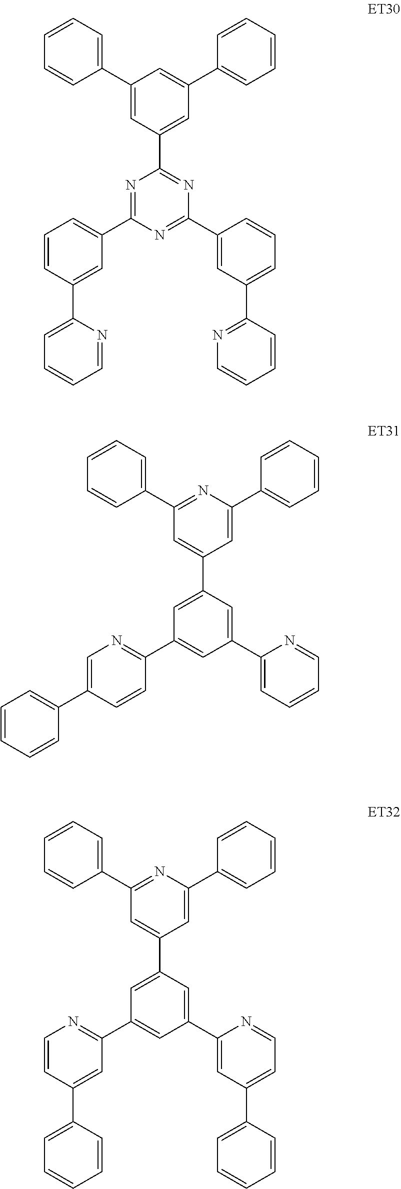 [SMILES: c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1]

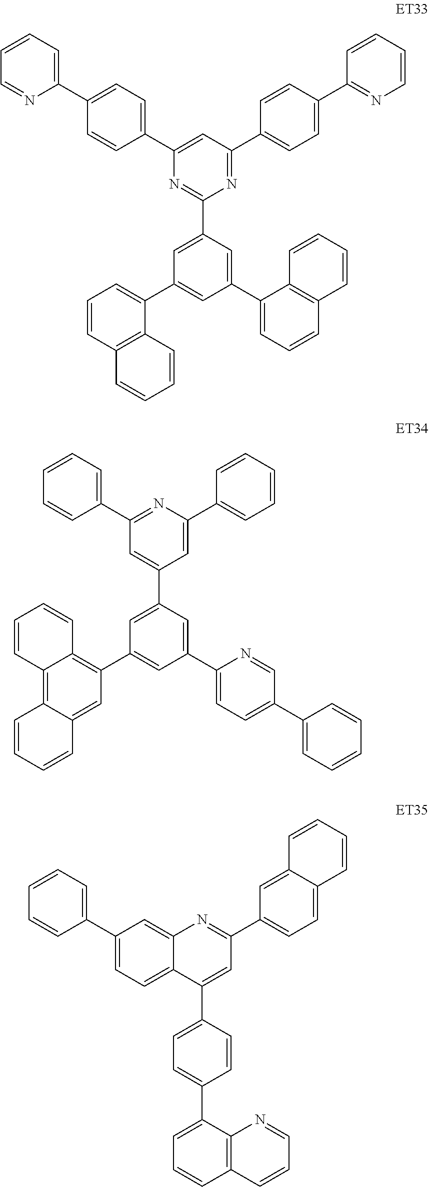 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1]

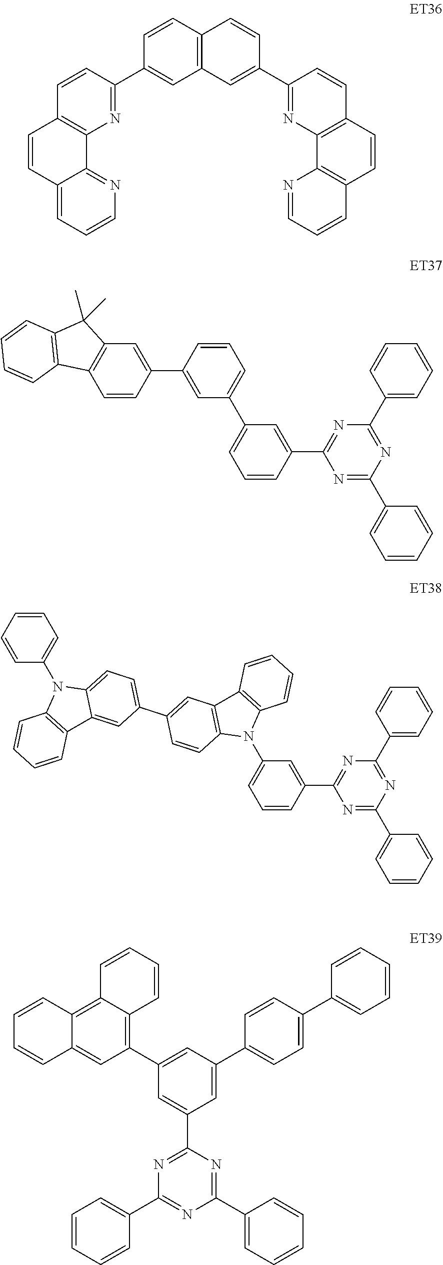 [SMILES: c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1]

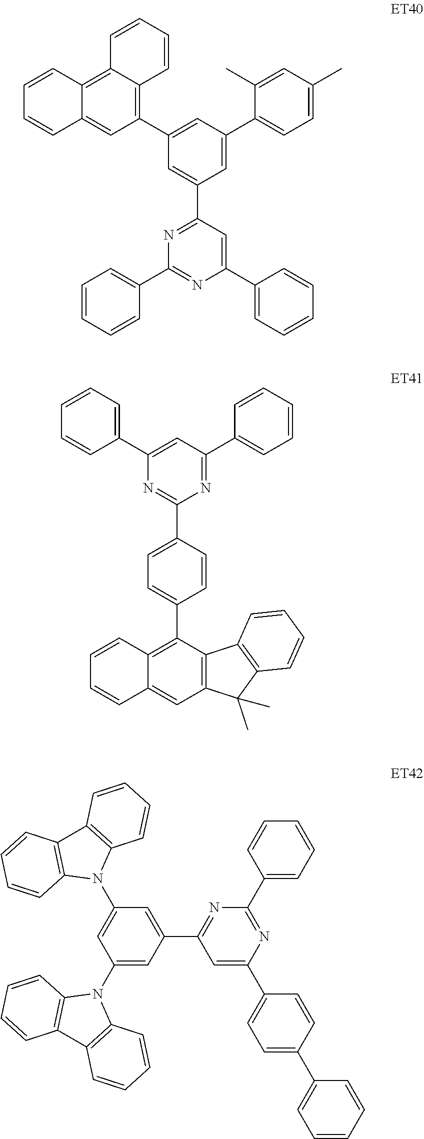 [SMILES: Cc1ccc(-c2cc(-c3cc(-c4ccccc4)nc(-c4ccccc4)n3)cc(-c3cc4ccccc4c4ccccc34)c2)c(C)c1 ; CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

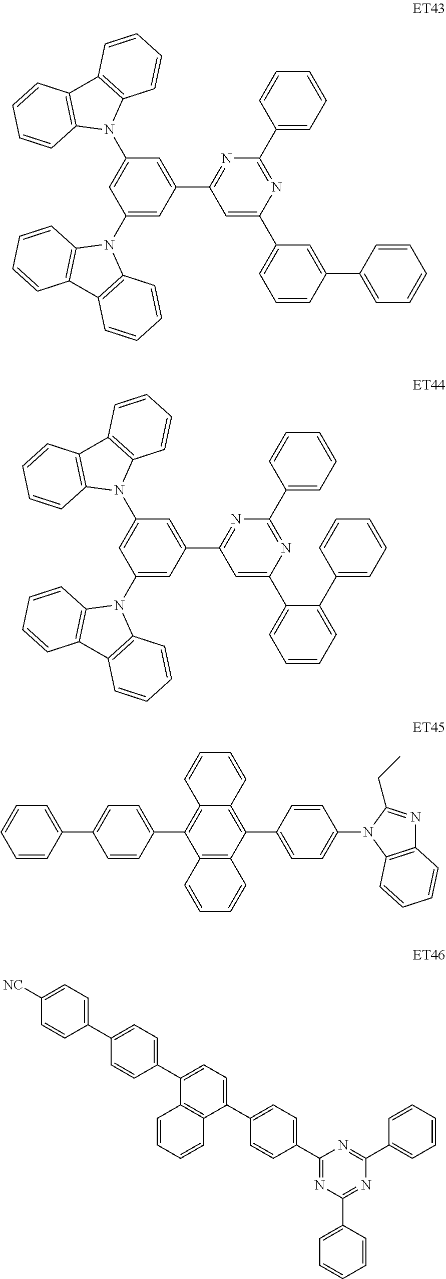 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1 ; N#Cc1ccc(-c2ccc(-c3ccc(-c4ccc(-c5nc(-c6ccccc6)nc(-c6ccccc6)n5)cc4)c4ccccc34)cc2)cc1]

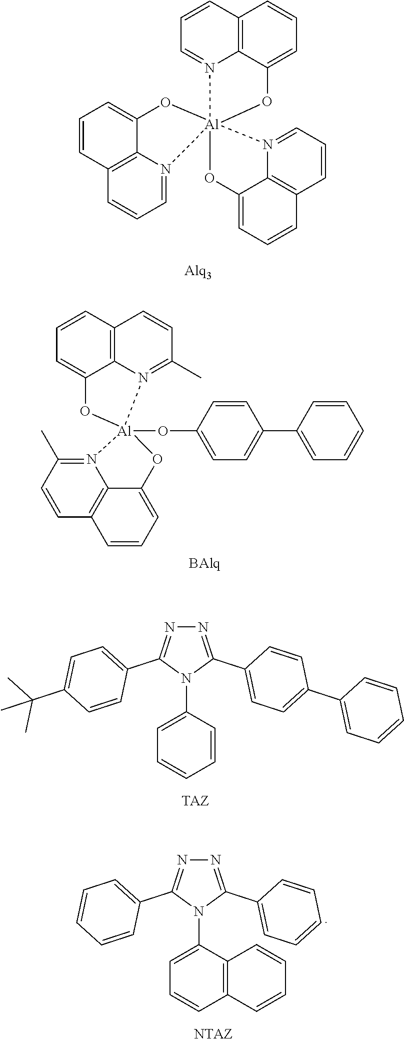

The thickness of the electron transport region may be in a range of about 100 Å to about 5,000 Å, for example, about 160 Å to about 4,000 Å. When the electron transport region includes a buffer layer, a hole blocking layer, an electron control layer, an electron transport layer, or any combination thereof, the thickness of the buffer layer, the hole blocking layer, or the electron control layer may be in a range of about 20 Å to about 1,000 Å, for example, about 30 Å to about 300 Å, and the thickness of the electron transport layer may be in a range of about 100 Å to about 1,000 Å, for example, about 150 Å to about 500 Å. When the thickness of the buffer layer, the hole-blocking layer, the electron control layer, the electron transport layer, and/or the electron transport layer are within these ranges, satisfactory electron transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth metal complex, or any combination thereof. The metal ion of an alkali metal complex may be a Li ion, a Na ion, a K ion, a Rb ion, or a Cs ion, and the metal ion of an alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, the metal-containing material may include a Li complex. The Li complex may include, for example, Compound ET-D1 (LiQ) or ET-D2:

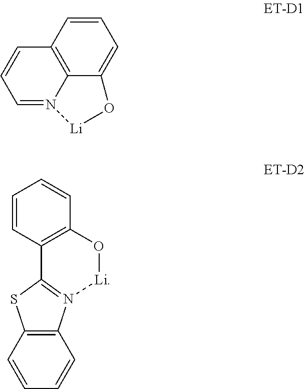

The electron transport region may include an electron injection layer that facilitates the injection of electrons from the second electrode 150. The electron injection layer may directly contact the second electrode 150.

The electron injection layer may have: i) a single-layer structure including (e.g., consisting of) a single layer including (e.g., consisting of) a single material, ii) a single-layer structure including (e.g., consisting of) a single layer consisting of multiple different materials, or iii) a multi-layer structure including multiple layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, iodides, etc.), or tellurides of the alkali metal, the alkaline earth metal, and the rare earth metal, or any combination thereof.

The alkali metal-containing compound may include: alkali metal oxides, such as Li₂O, Cs₂O, or K₂O; alkali metal halides, such as LiF, NaF, CsF, KF, LiI, NaI, CsI, KI, or RbI; or any combination thereof. The alkaline earth metal-containing compound may include an alkaline earth metal compound, such as BaO, SrO, CaO, BaₓSr₁₋ₓO (wherein x is a real number satisfying 0<x<1), BaₓCa₁₋ₓO (wherein x is a real number satisfying 0<x<1), and/or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal telluride. Examples of the lanthanide metal telluride are LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and/or the like.

The alkali metal complex, the alkaline earth-metal complex, and the rare earth metal complex may include i) one of selected from among metal ions of the alkali metal, the alkaline earth metal, and the rare earth metal and ii), as a ligand bonded to the metal ion, for example, a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenyl benzimidazole, a hydroxyphenylbenzothiazole, a bipyridine, a phenanthroline, a cyclopentadiene, or any combination thereof.

In one or more embodiments, the electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (for example, a compound represented by Formula 601).

In one or more embodiments, the electron injection layer may include (e.g., consist of)
i) an alkali metal-containing compound (for example, alkali metal halide),
ii) a) an alkali metal-containing compound (for example, alkali metal halide) and b) an alkali metal, an alkaline earth metal, a rare earth metal, or any combination thereof. For example, the electron injection layer may be a KI:Yb co-deposited layer, an RbI:Yb co-deposited layer, a LiF:Yb co-deposited layer, and/or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be uniformly or non-uniformly dispersed in a matrix including the organic material.

The thickness of the electron injection layer may be in a range of about 1 Å to about 100 Å, and, for example, about 3 Å to about 90 Å. When the thickness of the electron injection layer is within the ranges above, satisfactory electron injection characteristics may be obtained without a substantial increase in driving voltage.

### Second Electrode 150

Returning to FIG. 1, the second electrode 150 may be arranged on the interlayer 130 having a structure as described herein. The second electrode 150 may be a cathode, which is an electron injection electrode, and as a material for forming the second electrode 150, a metal, an alloy, an electrically conductive compound, or any combination thereof, each having a low work-function, may be utilized. The term “low work-function material” as utilized herein refers to a substance (e.g., a metal or metal alloy) that requires a relatively small amount of energy to emit electrons from its surface.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layer structure or a multi-layer structure including multiple layers.

### Capping Layer

The first capping layer may be arranged outside the first electrode 110, and/or the second capping layer may be arranged outside the second electrode 150. In particular, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated in the emission layer of the interlayer 130 of the light-emitting device 10 may be extracted toward the outside through the first electrode 110 which is a semi-transmissive electrode or a transmissive electrode, and the first capping layer. Light generated in the emission layer of the interlayer 130 of the light-emitting device 10 may be extracted toward the outside through the second electrode 150 which is a semi-transmissive electrode or a transmissive electrode, and the second capping layer.

The first capping layer and the second capping layer may increase external emission efficiency according to the principle of constructive interference. Accordingly, the light extraction efficiency of the light-emitting device 10 is increased, so that the luminescence efficiency of the light-emitting device 10 may be enhanced or improved.

Each of the first capping layer and the second capping layer may include a material having a refractive index of greater than or equal to 1.6 (at 589 nm).

The first capping layer and the second capping layer may each independently be an organic capping layer including an organic material, an inorganic capping layer including an inorganic material, or an organic-inorganic composite capping layer including an organic material and an inorganic material.

At least one of selected from among the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may optionally be substituted with a substituent including O, N, S, Se, Si, F, Cl, Br, I, or any combination thereof.

In one or more embodiments, at least one of selected from among the first capping layer and the second capping layer may each independently include an amine group-containing compound.

In one or more embodiments, at least one of selected from among the first capping layer and the second capping layer may each independently include a compound represented by Formula 201, a compound represented by Formula 202, or any combination thereof.

In one or more embodiments, at least one of selected from among the first capping layer and the second capping layer may each independently include: one of selected from among Compounds HT28 to HT33; Compounds CP1 to CP6; β-NPB; and/or any combination thereof:

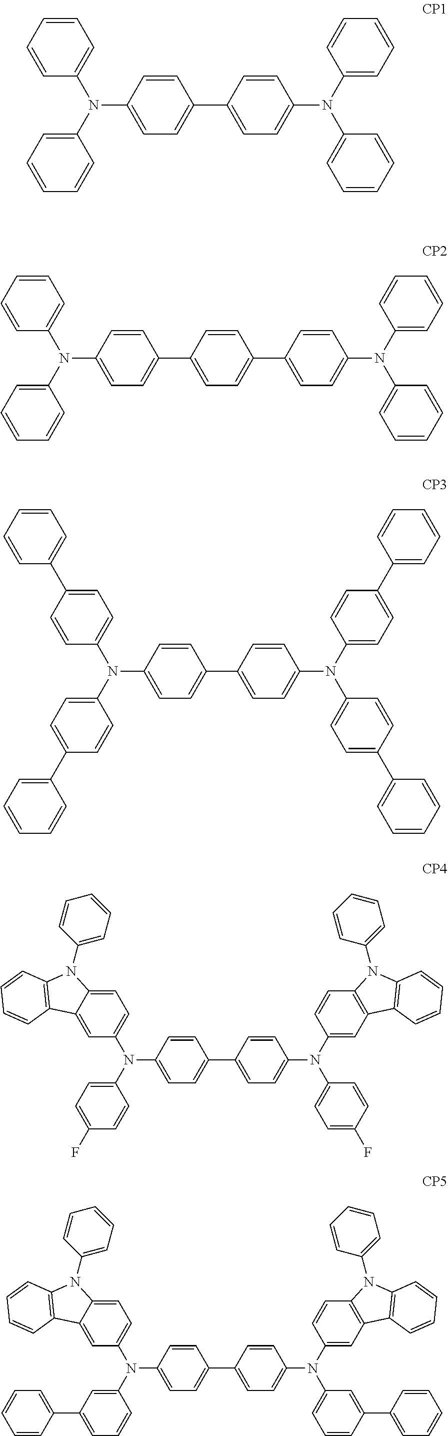 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

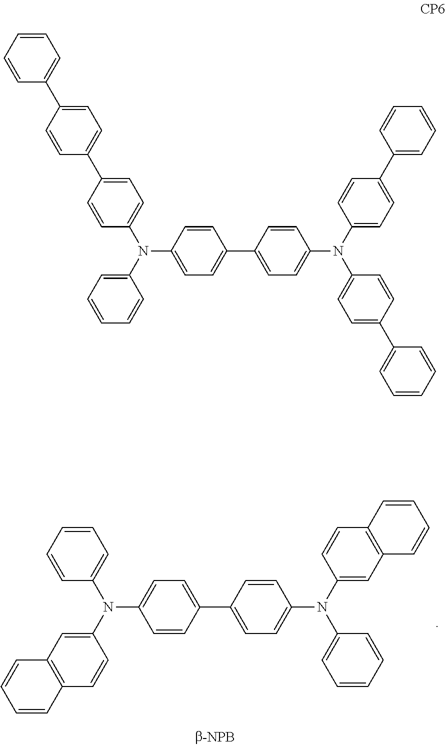 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Electronic Apparatus

The light-emitting device may be included in one or more suitable electronic apparatuses. For example, the electronic apparatus including the light-emitting device may be a light-emitting apparatus, an authentication apparatus, and/or the like.

The electronic apparatus (for example, a light-emitting apparatus) may further include, in addition to the light-emitting device, i) a color filter, ii) a color conversion layer, or iii) a color filter and a color conversion layer. The color filter and/or the color conversion layer may be arranged in at least one traveling direction of light emitted from the light-emitting device. For example, the light emitted from the light-emitting device may be blue light, green light, or white light. Details on the light-emitting device may be referred to the descriptions provided herein. In one or more embodiments, the color conversion layer may include a quantum dot.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be arranged among the subpixel areas to define each of the subpixel areas.

The color filter may further include a plurality of color filter areas and light-shielding patterns arranged among the color filter areas, and the color conversion layer may further include a plurality of color conversion areas and light-shielding patterns arranged among the color conversion areas.

The plurality of color filter areas (or the plurality of color conversion areas) may include a first area emitting first color light, a second area emitting second color light, and/or a third area emitting third color light, wherein the first color light, the second color light, and/or the third color light may have different maximum emission wavelengths from one another. For example, the first color light may be red light, the second color light may be green light, and the third color light may be blue light. For example, the plurality of color filter areas (or the plurality of color conversion areas) may include quantum dots. In particular, the first area may include a red quantum dot, the second area may include a green quantum dot, and the third area may not include (e.g., may exclude) a quantum dot. Details on the quantum dot may be referred to the descriptions provided herein. The first area, the second area, and/or the third area may each further include a scatter.

For example, the light-emitting device may be to emit first light, the first area may be to absorb the first light to emit first-first color light, the second area may be to absorb the first light to emit second-first color light, and the third area may be to absorb the first light to emit third-first color light. Here, the first-first color light, the second-first color light, and the third-first color light may have different maximum emission wavelengths. In particular, the first light may be blue light, the first-first color light may be red light, the second-first color light may be green light, and the third-first color light may be blue light.

The electronic apparatus may further include a thin-film transistor, in addition to the light-emitting device as described above. The thin-film transistor may include a source electrode, a drain electrode, and an activation layer, wherein any one selected from among the source electrode and the drain electrode may be electrically connected to any one selected from among the first electrode and the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

The electronic apparatus may further include a sealing portion for sealing the light-emitting device. The sealing portion may be arranged between the color filter and/or the color conversion layer and the light-emitting device. The sealing portion allows light from the light-emitting device to be extracted to the outside, and concurrently (e.g., simultaneously) prevents ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin film encapsulation layer, the electronic apparatus may be flexible.

One or more functional layers may be additionally arranged on the sealing portion, in addition to the color filter and/or the color conversion layer, according to the utilize of the electronic apparatus. Examples of the functional layers may include a touch screen layer, a polarizing layer, and/or the like. The touch screen layer may be a pressure-sensitive touch screen layer, a capacitive touch screen layer, or an infrared touch screen layer. The authentication apparatus may be, for example, a biometric authentication apparatus that authenticates an individual by utilizing biometric information of a living body (for example, fingertips, pupils, etc.).

The authentication apparatus may further include, in addition to the light-emitting device as described above, a biometric information collector.

The electronic apparatus may be applied to one or more suitable displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (for example, meters for a vehicle, an aircraft, and a vessel), projectors, and/or the like.

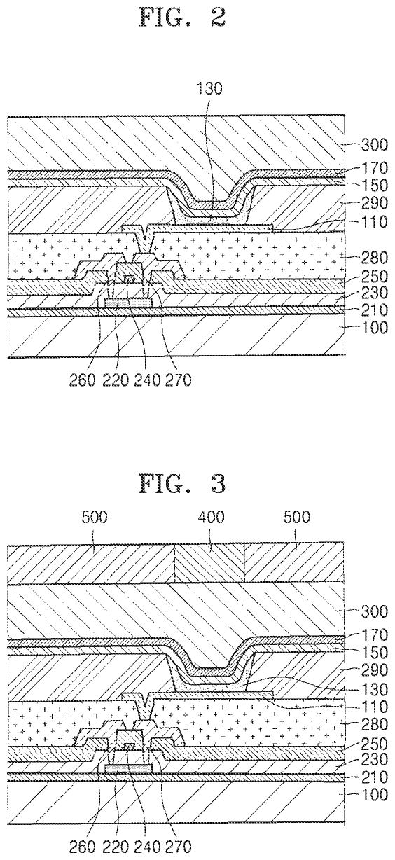

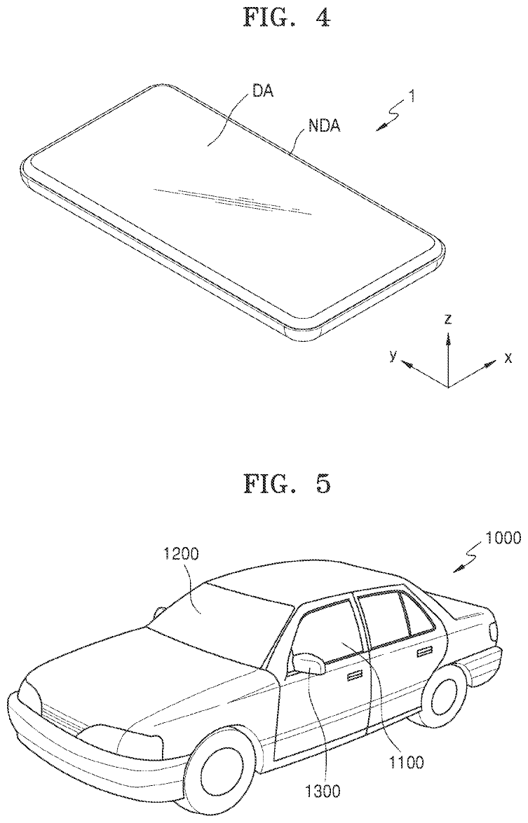

### Description of FIGS. 2 and 3

FIG. 2 is a cross-sectional view showing a light-emitting apparatus, which is an electronic apparatus according to one or more embodiments.

The light-emitting apparatus of FIG. 2 includes a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be located or arranged on the substrate 100. The buffer layer 210 may prevent or reduce penetration of impurities through the substrate 100 and may provide a flat surface on the substrate 100.

The TFT may be arranged on the buffer layer 210. The TFT may include an activation layer 220, a gate electrode 240, a source electrode 260, and a drain electrode 270.

The activation layer 220 may include an inorganic semiconductor such as silicon or polysilicon, an organic semiconductor, or an oxide semiconductor, and may include a source region, a drain region, and a channel region.

A gate insulating film 230 for insulating the activation layer 220 from the gate electrode 240 may be located or arranged on the activation layer 220, and the gate electrode 240 may be located or arranged on the gate insulating film 230.

An interlayer insulating film 250 may be located or arranged on the gate electrode 240. The interlayer insulating film 250 may be located or arranged between the gate electrode 240 and the source electrode 260 and between the gate electrode 240 and the drain electrode 270, to insulate from one another.

The source electrode 260 and the drain electrode 270 may be located or arranged on the interlayer insulating film 250. The interlayer insulating film 250 and the gate insulating film 230 may be formed to expose the source region and the drain region of the activation layer 220, and the source electrode 260 and the drain electrode 270 may be located or arranged in contact with the exposed portions of the source region and the drain region of the activation layer 220.

The TFT may be electrically connected to a light-emitting device to drive the light-emitting device, and may be covered and protected by a passivation layer 280. The passivation layer 280 may include an inorganic insulating film, an organic insulating film, or any combination thereof. A light-emitting device may be provided on the passivation layer 280. The light-emitting device may include the first electrode 110, the interlayer 130, and the second electrode 150.

The first electrode 110 may be located or arranged on the passivation layer 280. The passivation layer 280 may be located or arranged to expose a portion of the drain electrode 270, not fully covering the drain electrode 270, and the first electrode 110 may be located or arranged to be connected to the exposed portion of the drain electrode 270.

A pixel defining layer 290 including an insulating material may be located or arranged on the first electrode 110. The pixel defining layer 290 may expose a certain region of the first electrode 110, and an interlayer 130 may be formed in the exposed region of the first electrode 110. The pixel defining layer 290 may be a polyimide-based organic film or a polyacrylic-based organic film. In one or more embodiments, at least some layers of the interlayer 130 may extend beyond the upper portion of the pixel defining layer 290 to be located or arranged in the form of a common layer.

The second electrode 150 may be located on the interlayer 130, and a second capping layer 170 may be additionally formed on the second electrode 150. The second capping layer 170 may be formed to cover the second electrode 150.

The encapsulation portion 300 may be located on the second capping layer 170. The encapsulation portion 300 may be located or arranged on a light-emitting device to protect the light-emitting device from moisture and/or oxygen. The encapsulation portion 300 may include: an inorganic film including silicon nitride (SiNx), silicon oxide (SiOx), indium tin oxide (ITO), indium zinc oxide (IZO), or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and/or the like), an epoxy-based resin (for example, aliphatic glycidyl ether (AGE), and/or the like), or any combination thereof; or any combination of the inorganic film(s) and the organic film(s).

FIG. 3 is a cross-sectional view showing a light-emitting apparatus, which is an electronic apparatus according to one or more embodiments.

The light-emitting apparatus of FIG. 3 is the same as the light-emitting apparatus of FIG. 2, except that a light-shielding pattern 500 and a functional region 400 are additionally located or arranged on the encapsulation portion 300. The functional region 400 may be i) a color filter area, ii) a color conversion area, or iii) a combination of the color filter area and the color conversion area. In one or more embodiments, the light-emitting device included in the light-emitting apparatus of FIG. 3 may be, or may be referred to as, a tandem light-emitting device.

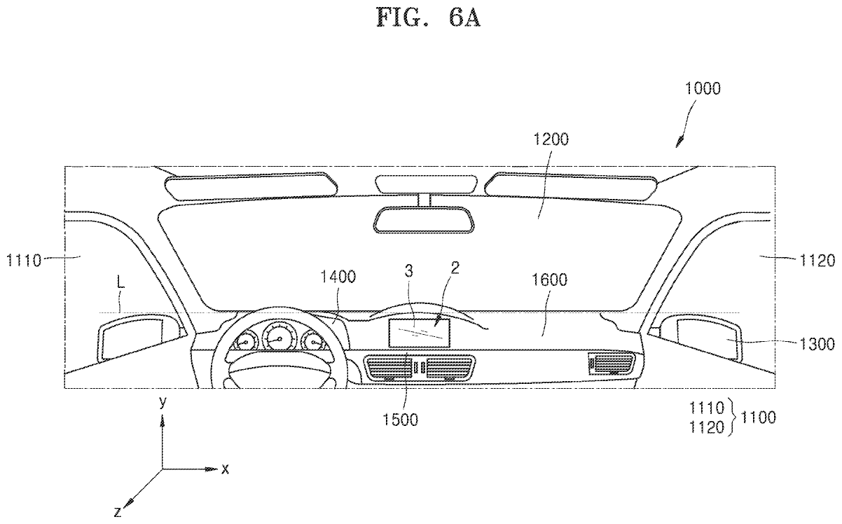

### Description of FIG. 4

FIG. 4 is a schematic perspective view of an electronic equipment 1 including a light-emitting device according to one or more embodiments. The electronic equipment 1 may be, as a device apparatus that displays a moving image or still image, a portable electronic equipment, such as a mobile phone, a smart phone, a tablet personal computer (PC), a mobile communication terminal, an electronic notebook, an electronic book, a portable multimedia player (PMP), a navigation, or a ultra mobile PC (UMPC), as well as one or more suitable products, such as a television, a laptop, a monitor, a billboards, or an Internet of things (IOT). The electronic equipment 1 may be such a product, as described herein, or a component or part thereof. In some embodiments, the electronic equipment 1 may be a wearable device, such as a smart watch, a watch phone, a glasses-type or kind display, or a head mounted display (HMD), or a part of the wearable device. However, embodiments of the disclosure are not limited thereto. For example, the electron equipment 1 may include a dashboard of a vehicle, a center fascia of a vehicle, a center information display arranged on a dashboard of a vehicle, a room mirror display replacing a side mirror of a vehicle, an entertainment display for the rear seat of a vehicle or a display arranged on the back of the front seat, or a head up display (HUD) installed in the front of a vehicle or projected on a front window glass, a computer generated hologram augmented reality head up display (CGH AR HUD). FIG. 4 illustrates a case in which the electronic equipment 1 is a smart phone for convenience of explanation.

The electronic equipment 1 may include a display area DA and a non-display area NDA outside the display area DA. A display device may implement an image through an array of a plurality of pixels that are two-dimensionally arranged in the display area DA.

The non-display area NDA is an area that does not display an image, and may entirely surround the display area DA. On the non-display area NDA, a driver for providing electrical signals and/or power to display devices located or arranged on the display area DA may be located or arranged. On the non-display area NDA, a pad, which is an area to which an electronic element or a printing circuit board, may be electrically connected may be located or arranged.

In the electronic equipment 1, a length in the x-axis direction and a length in the y-axis direction may be different from each other. In one or more embodiments, as shown in FIG. 4, the length in the x-axis direction may be shorter than the length in the y-axis direction. In one or more embodiments, the length in the x-axis direction may be the same as the length in the y-axis direction. In one or more embodiments, the length in the x-axis direction may be longer than the length in the y-axis direction.

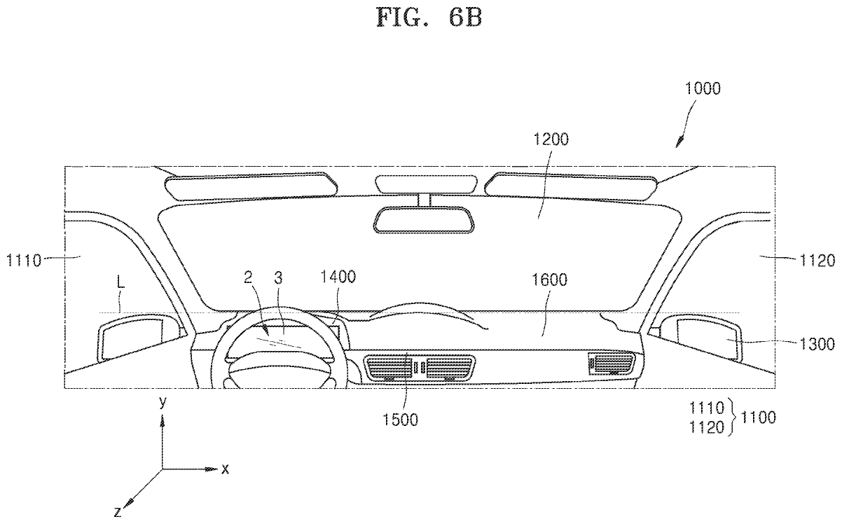

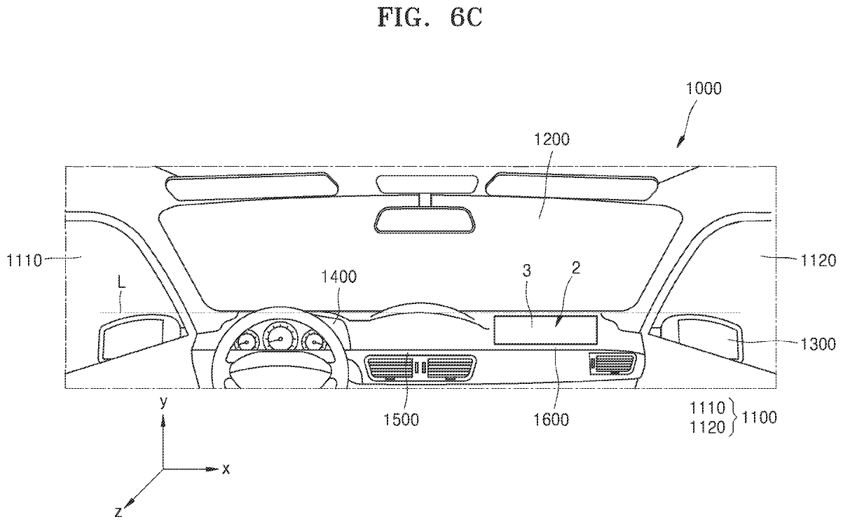

### Descriptions of FIGS. 5 and 6A to 6C

FIG. 5 is a schematic perspective view of the exterior of a vehicle 1000 according to one or more embodiments. FIGS. 6A to 6C are each a schematic view of the interior of a vehicle 1000 that includes the electronic equipment including a light-emitting device according to one or more suitable embodiments.

Referring to FIGS. 5, 6A, 6B, and 6C, the vehicle 1000 may refer to one or more suitable apparatus(es) for moving a subject to be transported, such as a human, an object, and/or an animal, from a departure point to a destination point. The vehicle 1000 may include a vehicle (e.g., automobile) traveling on a road or track, a vessel moving over the sea or river, an airplane flying in the sky utilizing the action of air, and/or the like.

The vehicle 1000 may travel on a road or a track. The vehicle 1000 may move in a set or predetermined direction according to rotation of at least one wheel. For example, the vehicle 1000 may include a three-wheeled or four-wheeled vehicle, a construction machine, a two-wheeled vehicle, a prime mover device, a bicycle, and a train running on a track.

The vehicle 1000 may include a body having an interior and an exterior, and a chassis in which mechanical apparatuses necessary for driving are installed as other parts except for the body. The exterior of the body may include a front panel, a bonnet (i.e., a hood), a roof panel, a rear panel, a trunk, a pillar provided at a boundary between doors, and/or the like. The chassis of the vehicle 1000 may include a power generating device, a power transmitting device, a driving device, a steering device, a braking device, a suspension device, a transmission device, a fuel device, front and rear left and right wheels, and/or the like.

The vehicle 1000 may include a side window glass 1100, a front window glass 1200, a side mirror 1300, a cluster 1400, a center fascia 1500, a passenger seat dashboard 1600, and a display device 2.

The side window glass 1100 and the front window glass 1200 may be partitioned by a pillar arranged between the side window glass 1100 and the front window glass 1200.

The side window glass 1100 may be installed on the side of the vehicle 1000. In one or more embodiments, the side window glass 1100 may be installed on a door of the vehicle 1000. A plurality of side window glasses 1100 may be provided and may face each other. In one or more embodiments, the side window glass 1100 may include a first side window glass 1110 and a second side window glass 1120. In one or more embodiments, the first side window glass 1110 may be arranged adjacent to the cluster 1400. The second side window glass 1120 may be arranged adjacent to the passenger seat dashboard 1600.

In one or more embodiments, the side window glasses 1100 may be spaced apart from each other in the +x direction or the −x direction. For example, the first side window glass 1110 and the second side window glass 1120 may be spaced apart from each other in the +x direction or the −x direction. In other words, an imaginary straight line L connecting the side window glasses 1100 may extend in the +x direction or the −x direction. For example, an imaginary straight line L connecting the first side window glass 1110 and the second side window glass 1120 to each other may extend in the +x direction or the −x direction.

The front window glass 1200 may be installed in front of the vehicle 1000. The front window glass 1200 may be arranged between the side window glasses 1100 facing each other.

The side mirror 1300 may provide a rear view of the vehicle 1000. The side mirror 1300 may be installed on the exterior of the vehicle body. In one embodiment, a plurality of side mirrors 1300 may be provided. Any one of selected from among the plurality of side mirrors 1300 may be arranged outside the first side window glass 1110. The other one of selected from among the plurality of side mirrors 1300 may be arranged outside the second side window glass 1120.

The cluster 1400 may be arranged in front of the steering wheel. The cluster 1400 may include a tachometer, a speedometer, a coolant thermometer, a fuel gauge turn indicator, a high beam indicator, a warning lamp, a seat belt warning lamp, an odometer, an odometer, an automatic shift selector indicator lamp, a door open warning lamp, an engine oil warning lamp, and/or a low fuel warning light.

The center fascia 1500 may include a control panel on which a plurality of buttons for adjusting an audio device, an air conditioning device, and a heater of a seat are disposed. The center fascia 1500 may be arranged on one side of the cluster 1400.

A passenger seat dashboard 1600 may be spaced apart from the cluster 1400 with the center fascia 1500 arranged therebetween. In one or more embodiments, the cluster 1400 may be arranged to correspond to a driver seat, and the passenger seat dashboard 1600 may be disposed to correspond to a passenger seat. In one or more embodiments, the cluster 1400 may be adjacent to the first side window glass 1110, and the passenger seat dashboard 1600 may be adjacent to the second side window glass 1120.

In one or more embodiments, the display device 2 may include a display panel 3, and the display panel 3 may display an image. The display device 2 may be arranged inside the vehicle 1000. In one or more embodiments, the display device 2 may be arranged between the side window glasses 1100 facing each other. The display device 2 may be arranged on at least one of selected from among the cluster 1400, the center fascia 1500, and the passenger seat dashboard 1600.

The display device 2 may include an organic light-emitting display device, an inorganic EL display device, a quantum dot display device, and/or the like. Hereinafter, as the display device 2 according to one or more embodiments of the present disclosure, an organic light-emitting display device including the light-emitting device according to the present disclosure will be described as an example, but one or more suitable types (kinds) of display devices as described above may be utilized in embodiments of the present disclosure.

Referring to FIG. 6A, the display device 2 may be located or arranged on the center fascia 1500. In one or more embodiments, the display device 2 may display navigation information. In one or more embodiments, the display device 2 may display audio, video, or information regarding vehicle settings.

Referring to FIG. 6B, the display device 2 may be arranged on the cluster 1400. When the display device 2 is arranged on the cluster 1400, the cluster 1400 may display driving information and/or the like through the display device 2. For example, the cluster 1400 may be implemented digitally. The digital cluster 1400 may display vehicle information and driving information as images. For example, a needle and a gauge of a tachometer and one or more suitable warning light icons may be displayed by a digital signal.

Referring to FIG. 6C, the display device 2 may be arranged on the passenger seat dashboard 1600. The display device 2 may be embedded in the passenger seat dashboard 1600 or arranged on the passenger seat dashboard 1600. In one or more embodiments, the display device 2 arranged on the dashboard 1600 for the passenger seat may display an image related to information displayed on the cluster 1400 and/or information displayed on the center fascia 1500. In one or more embodiments, the display device 2 arranged on the passenger seat dashboard 1600 may display information different from information displayed on the cluster 1400 and/or information displayed on the center fascia 1500.

### Manufacturing Method

Respective layers included in the hole transport region, the emission layer, and respective layers included in the electron transport region may be formed in one or more suitable regions by utilizing one or more suitable methods selected from vacuum deposition, spin coating, casting, Langmuir-Blodgett (LB) deposition, ink-jet printing, laser-printing, laser-induced thermal imaging, and/or the like.

When respective layers included in the hole transport region, the emission layer, and respective layers included in the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 angstrom per second (Å/sec) to about 100 Å/sec, depending on a material to be included in a layer to be formed and the structure of a layer to be formed. In one or more embodiments, the deposition may be performed at a deposition temperature of about 180° C. to about 340° C.

### Definition of Terms

The term “C₃-C₆₀ carbocyclic group” as utilized herein refers to a cyclic group consisting of carbon only as a ring-forming atom and having three to sixty carbon atoms, and the term “C₁-C₆₀ heterocyclic group” as utilized herein refers to a cyclic group that has one to sixty carbon atoms and further has, in addition to carbon, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. For example, the number of ring-forming atoms of the C₁-C₆₀ heterocyclic group may be from 3 to 61.

The “cyclic group” as utilized herein may include both (e.g., simultaneously) the C₃-C₆₀ carbocyclic group and the C₁-C₆₀ heterocyclic group.

The term “π electron-rich C₃-C₆₀ cyclic group” as utilized herein refers to a cyclic group that has three to sixty carbon atoms and does not include *—N═*′ as a ring-forming moiety, and the term “π electron-deficient nitrogen-containing C₁-C₆₀ heterocyclic group” as utilized herein refers to a heterocyclic group that has one to sixty carbon atoms and includes *—N═*′ as a ring-forming moiety.

For example, the C₃-C₆₀ carbocyclic group may be i) a T1 group or ii) a condensed cyclic group in which two or more T1 groups are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),

the C₁-C₆₀ heterocyclic group may be i) a T2 group, ii) a condensed cyclic group in which at least two T2 groups are condensed with each other, or iii) a condensed cyclic group in which at least one T2 group and at least one T1 group are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),

the π electron-rich C₃-C₆₀ cyclic group may be i) a T1 group, ii) a condensed cyclic group in which at least two T1 groups are condensed with each other, iii) a T3 group, iv) a condensed cyclic group in which at least two T3 groups are condensed with each other, or v) a condensed cyclic group in which at least one T3 group and at least one T1 group are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, and/or the like),

the π electron-deficient nitrogen-containing C₁-C₆₀ heterocyclic group may be i) a T4 group, ii) a condensed cyclic group in which at least two T4 groups are condensed with each other, iii) a condensed cyclic group in which at least one T4 group and at least one T1 group are condensed with each other, iv) a condensed cyclic group in which at least one T4 group and at least one T3 group are condensed with each other, or v) a condensed cyclic group in which at least one T4 group, at least one T1 group, and at least one T3 group are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),

the T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group,

the group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a petrahydropyrazine group, a dihydropyrazine group, a tetrahydropyridazine group, or a dihydropyridazine group,

the T3 group may be a furan group, a thiophene group, a 1H-pyrrole group, a silole group, or a borole group, and

the T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group, the C₃-C₆₀ carbocyclic group, the C₁-C₆₀ heterocyclic group, the π electron-rich C₃-C₆₀ cyclic group, or the π electron-deficient nitrogen-containing C₁-C₆₀ heterocyclic group” as utilized herein refer to a group condensed to any cyclic group, a monovalent group, or a polyvalent group (for example, a divalent group, a trivalent group, a tetravalent group, etc.) according to the structure of a formula for which the corresponding term is utilized. In one or more embodiments, “a benzene group” may be a benzo group, a phenyl group, a phenylene group, and/or the like, which may be easily understand by one of ordinary skill in the art according to the structure of a formula including the “benzene group.”

Depending on context, a divalent group may refer or be a polyvalent group (e.g., trivalent, tetravalent, etc., and not just divalent) per, e.g., the structure of a formula in connection with which of the terms are utilized.

Examples of the monovalent C₃-C₆₀ carbocyclic group and the monovalent C₁-C₆₀ heterocyclic group are a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Examples of the divalent C₃-C₆₀ carbocyclic group and the monovalent C₁-C₆₀ heterocyclic group are a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group” as utilized herein refers to a linear or branched aliphatic hydrocarbon monovalent group that has one to sixty carbon atoms, and specific examples thereof are a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a see-decyl group, and a tert-decyl group. The term “C₁-C₆₀ alkylene group” as utilized herein refers to a divalent group having the same structure as the C₁-C₆₀ alkyl group.

The term “C₂-C₆₀ alkenyl group” as utilized herein refers to a monovalent hydrocarbon group having at least one carbon-carbon double bond in the middle or at the terminus of the C₂-C₆₀ alkyl group, and examples thereof are an ethenyl group, a propenyl group, a butenyl group, and/or the like. The term “C₂-C₆₀ alkenylene group” as utilized herein refers to a divalent group having the same structure as the C₂-C₆₀ alkenyl group.

The term “C₂-C₆₀ alkynyl group” as utilized herein refers to a monovalent hydrocarbon group having at least one carbon-carbon triple bond in the middle or at the terminus of the C₂-C₆₀ alkyl group, and examples thereof are an ethynyl group, a propynyl group, and/or the like. The term “C₂-C₆₀ alkynylene group” as utilized herein refers to a divalent group having the same structure as the C₂-C₆₀ alkynyl group.

The term “C₁-C₆₀ alkoxy group” as utilized herein refers to a monovalent group represented by—OA₁₀₁ (wherein A₁₀₁ is the C₁-C₆₀ alkyl group), and examples thereof are a methoxy group, an ethoxy group, an isopropyloxy group, and/or the like.

The term “C₃-C₁₀ cycloalkyl group” as utilized herein refers to a monovalent saturated hydrocarbon cyclic group having 3 to 10 carbon atoms, and examples thereof are a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, a bicyclo[2.2.2]octyl group, and/or the like. The term “C₃-C₁₀ cycloalkylene group” as utilized herein refers to a divalent group having the same structure as the C₃-C₁₀ cycloalkyl group.

The term “C₁-C₁₀ heterocycloalkyl group” as utilized herein refers to a monovalent cyclic group of 1 to 10 carbon atoms, further including, in addition to carbon atoms, at least one heteroatom, as ring-forming atoms, and examples thereof are a 1,2,3,4-oxatriazolidinyl group, a tetrahydrofuranyl group, a tetrahydrothiophenyl group, and/or the like. The term “C₁-C₁₀ heterocycloalkylene group” as utilized herein refers to a divalent group having the same structure as the C₁-C₁₀ heterocycloalkyl group.

The term “C₃-C₁₀ cycloalkenyl group” as utilized herein refers to a monovalent cyclic group that has three to ten carbon atoms and at least one carbon-carbon double bond in the ring thereof and no aromaticity, and examples thereof are a cyclopentenyl group, a cyclohexenyl group, a cycloheptenyl group, and/or the like. The term “C₃-C₁₀ cycloalkenylene group” as utilized herein refers to a divalent group having the same structure as the C₃-C₁₀ cycloalkenyl group.

The term “C₁-C₁₀ heterocycloalkenyl group” as utilized herein refers to a monovalent cyclic group of 1 to 10 carbon atoms, further including, in addition to carbon atoms, at least one heteroatom, as ring-forming atoms, and having at least one carbon-carbon double bond in the cyclic structure thereof. Examples of the C₁-C₁₀ heterocycloalkenyl group are a 4,5-dihydro-1,2,3,4-oxatriazolyl group, a 2,3-dihydrofuranyl group, a 2,3-dihydrothiophenyl group, and/or the like. The term “C₁-C₁₀ heterocycloalkenylene group” as utilized herein refers to a divalent group having the same structure as the C₁-C₁₀ heterocycloalkenyl group.

The term “C₆-C₆₀ aryl group” as utilized herein refers to a monovalent group having a carbocyclic aromatic system of 6 to 60 carbon atoms, and the term “C₆-C₆₀ arylene group” as utilized herein refers to a divalent group having a carbocyclic aromatic system of 6 to 60 carbon atoms. Examples of the C₆-C₆₀ aryl group are a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and/or the like. When the C₆-C₆₀ aryl group and the C₆-C₆₀ arylene group each include two or more rings, the rings may be condensed with each other.

The term “C₁-C₆₀ heteroaryl group” as utilized herein refers to a monovalent group having a heterocyclic aromatic system of 1 to 60 carbon atoms, further including, in addition to carbon atoms, at least one heteroatom, as ring-forming atoms. The term “C₁-C₆₀ heteroarylene group” as utilized herein refers to a divalent group having a heterocyclic aromatic system of 1 to 60 carbon atoms, further including, in addition to carbon atoms, at least one heteroatom, as ring-forming atoms. Examples of the C₁-C₆₀ heteroaryl group are a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the rings may be condensed with each other.

The term “monovalent non-aromatic condensed polycyclic group” as utilized herein refers to a monovalent group (for example, having 8 to 60 carbon atoms) having two or more rings condensed to each other, only carbon atoms as ring-forming atoms, and no aromaticity in its entire molecular structure. Examples of the monovalent non-aromatic condensed polycyclic group are an indenyl group, a fluorenyl group, a spiro-bifluorenyl group, a benzofluorenyl group, an indenophenanthrenyl group, an indeno anthracenyl group, and/or the like. The term “divalent non-aromatic condensed polycyclic group” as utilized herein refers to a divalent group having the same structure as the monovalent non-aromatic condensed polycyclic group described above.

The term “monovalent non-aromatic condensed heteropolycyclic group” as utilized herein refers to a monovalent group (for example, having 1 to 60 carbon atoms) having two or more rings condensed to each other, further including, in addition to carbon atoms, at least one heteroatom, as ring-forming atoms, and having non-aromaticity in its entire molecular structure. Examples of the monovalent non-aromatic condensed heteropolycyclic group are a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group. The term “divalent non-aromatic condensed heteropolycyclic group” as utilized herein refers to a divalent group having the same structure as the monovalent non-aromatic condensed heteropolycyclic group described above.

The term “C₆-C₆₀ aryloxy group” as utilized herein indicates—OA₁₀₂ (wherein A₁₀₂ is the C₆-C₆₀ aryl group), and the term “C₆-C₆₀ arylthio group” as utilized herein indicates—SA₁₀₃ (wherein A₁₀₃ is the C₆-C₆₀ aryl group).

The term “C₇-C₆₀ arylalkyl group” as utilized herein refers to -A₁₀₄A₁₀₅ (wherein A₁₀₄ is a C₁-C₅₄ alkylene group, and A₁₀₅ is a C₆-C₅₉ aryl group), and the term “C₂-C₆₀ heteroarylalkyl group” as utilized herein refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as utilized herein may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃, and Q₃₁ to Q₃₃ in the specification may each independently be: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

The term “heteroatom” as utilized herein refers to any atom other than a carbon atom. Examples of the heteroatom are O, S, N, P, Si, B, Ge, Se, and any combination thereof.

In the specification, the third-row transition metal may include hafnium (Hf), tantalum (Ta), tungsten (W), rhenium (Re), osmium (Os), iridium (Ir), platinum (Pt), gold (Au), and/or the like.

The term “Ph” as utilized herein refers to a phenyl group, the term “Me” as utilized herein refers to a methyl group, the term “Et” as utilized herein refers to an ethyl group, the term “ter-Bu” or “Bu^{t}” as utilized herein refers to a tert-butyl group, and the term “OMe” as utilized herein refers to a methoxy group.

The term “biphenyl group” as utilized herein refers to “a phenyl group substituted with a phenyl group.” In other words, the “biphenyl group” is a substituted phenyl group having a C₆-C₆₀ aryl group as a substituent.

The term “terphenyl group” as utilized herein refers to “a phenyl group substituted with a biphenyl group”. In other words, the “terphenyl group” is a substituted phenyl group having, as a substituent, a C₆-C₆₀ aryl group substituted with a C₆-C₆₀ aryl group.

The symbols * and *′ as utilized herein, unless defined otherwise, each refer to a binding site to a neighboring atom in a corresponding formula or moiety.

In the present specification, the x-axis, y-axis, and z-axis are not limited to three axes in an orthogonal coordinate system, and may be interpreted in a broad sense including these axes. For example, the x-axis, y-axis, and z-axis may refer to those orthogonal to each other, or may refer to those in different directions that are not orthogonal to each other.

The term “substituted” as utilized herein, refers to that at least one hydrogen in a substituent or compound is deuterium, a halogen group, a hydroxyl group, an amino group, a substituted or unsubstituted C₁ to C₃₀ amine group, a nitro group, a substituted or unsubstituted C₁ to C₄₀ silyl group, a C₁ to C₃₀ alkyl group, a C₁ to C₁₀ alkylsilyl group, a C₆ to C₃₀ arylsilyl group, a C₃ to C₃₀ cycloalkyl group, a C₃ to C₃₀ heterocycloalkyl group, a C₆ to C₃₀ aryl group, a C₂ to C₃₀ heteroaryl group, a C₁ to C₂₀ alkoxy group, a C₁ to C₁₀ fluoroalkyl group, a cyano group, or a combination thereof.

In one example of the present disclosure, “substituted” refers to replacement of at least one hydrogen of a substituent or a compound by deuterium, a halogen, a C₁ to C₃₀ alkyl group, a C₁ to C₁₀ alkylsilyl group, a C₆ to C₃₀ arylsilyl group, a C₃ to C₃₀ cycloalkyl group, a C₃ to C₃₀ heterocycloalkyl group, a C₆ to C₃₀ aryl group, a C₂ to C₃₀ heteroaryl group, a C₁ to C₁₀ fluoroalkyl group, or a cyano group. In some embodiments, in specific examples of the present disclosure, “substituted” refers to replacement of at least on hydrogen of a substituent or a compound by deuterium, a halogen, a C₁ to C₂₀ alkyl group, a C₆ to C₃₀ aryl group, a C₁ to C₁₀ fluoroalkyl group, or a cyano group. In some embodiments, in specific examples of the present disclosure, “substituted” refers to replacement of at least one hydrogen of a substituent or a compound by deuterium, a halogen, a C₁ to C₅ alkyl group, a C₆ to C₁₈ aryl group, a C₁ to C₅ fluoroalkyl group, or a cyano group.

In some embodiments, in specific examples of the present disclosure, “substituted” refers to replacement of at least one hydrogen of a substituent or a compound by deuterium, a cyano group, a halogen, a methyl group, an ethyl group, a propyl group, a butyl group, a phenyl group, a biphenyl group, a terphenyl group, a trifluoromethyl group, or a naphthyl group.

Hereinafter, compounds according to embodiments and light-emitting devices according to embodiments will be described in more detail with reference to the following synthesis examples and examples. The wording “B was utilized instead of A” utilized in describing Synthesis Examples refers to that an identical molar equivalent of B was utilized in place of A.

### EXAMPLES

### Synthesis Example 1: Synthesis of Compound H1

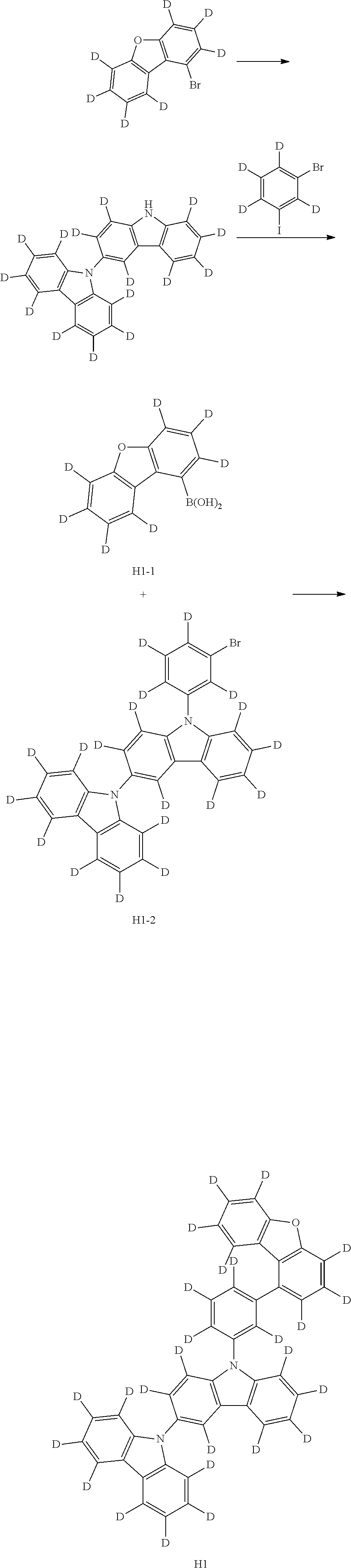

### (1) Synthesis of Intermediate H1-1

1-bromodibenzofuran-2,3,4,5,6,7,8-d7 (1 eq) was dissolved in THF and then contacted with n-butyllithium (1.2 eq) at −78° C. After 1 hour, trimethyl borate was added dropwise thereto. Intermediate H1-1 was obtained by slowly raising the temperature to room temperature. Intermediate H1-1 was identified by LC-MS as follows:

C₁₂H₂D₇BO₃ M+1 220.2.

### (2) Synthesis of Intermediate H1-2

9H-3,9′-bicarbazole-1,1′, 2,2′, 3′, 4,4′, 5,5′, 6,6′, 7,7′, 8,8′-d15 (1 eq), 1-bromo-3-iodobenzene-2,4,5,6-d4 (1.5 eq), and K₃PO₄ (2 eq) were dissolved in DMF, followed by stirring overnight at 160° C. to obtain Intermediate H1-2. Intermediate H1-2 was identified by LC-MS as follows:

C₃₀D₁₉BrN₂ M+1: 506.2.

### (3) Synthesis of Compound H1

1.5 g of Intermediate H1-1, 2.9 g of Intermediate H1-2, 0.3 g of tetrakis(triphenylphosphine)palladium, and 2 g of potassium carbonate were added to a reaction vessel, and dissolved in 100 mL of toluene, 20 mL of ethanol, and 20 mL of distilled water. The mixed solution was then refluxed for 24 hours. After the reaction was completed, the reaction solution was extracted with ethylacetate, the collected organic layer was dried with magnesium sulfate and a solvent was evaporated therefrom. The obtained residue was separated and purified by silica gel column chromatography to obtain 3 g (yield: 55%) of Compound H1. Compound H1 was identified by LC-MS, and shown in Table 1.

### Synthesis Example 2: Synthesis of Compound H3

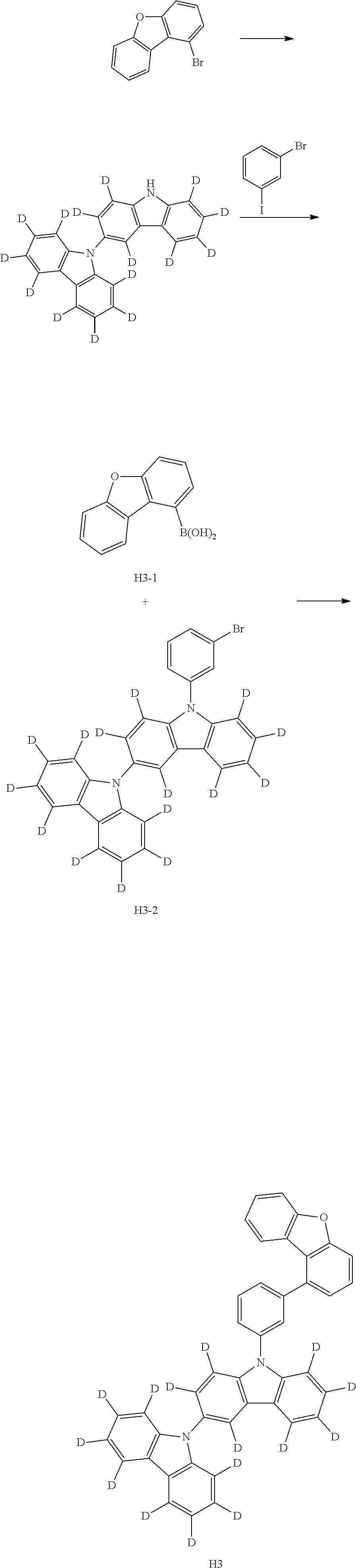

### (2) Synthesis of Intermediate H3-2

9H-3,9′-bicarbazole-1,1′, 2,2′, 3′, 4,4′, 5,5′, 6,6′, 7,7′, 8,8′-d15 (1 eq), 1-bromo-3-iodobenzene (1.5 eq), and K₃PO₄ (2 eq) were dissolved in DMF, followed by stirring overnight at 160° C. to obtain Intermediate H3-2. Intermediate H3-2 was identified by LC-MS as follows:

C₃₀H₄D₁₅BrN₂ M+1: 502.2.

### (3) Synthesis of Compound H3

1.4 g of Intermediate H3-1, 2.8 g of Intermediate H3-2, 0.3 g of tetrakis(triphenylphosphine)palladium, and 2 g of potassium carbonate were added to a reaction vessel, and dissolved in 100 mL of toluene, 20 mL of ethanol, and 20 mL of distilled water. The mixed solution was then refluxed for 24 hours. After the reaction was completed, the reaction solution was extracted with ethyl acetate, the collected organic layer was dried with magnesium sulfate and a solvent was evaporated therefrom. The obtained residue was separated and purified by silica gel column chromatography to obtain 2.8 g (yield: 55%) of Compound H3. Compound H3 was identified by LC-MS, and shown in Table 1.

### Synthesis Example 3: Synthesis of Compound H13

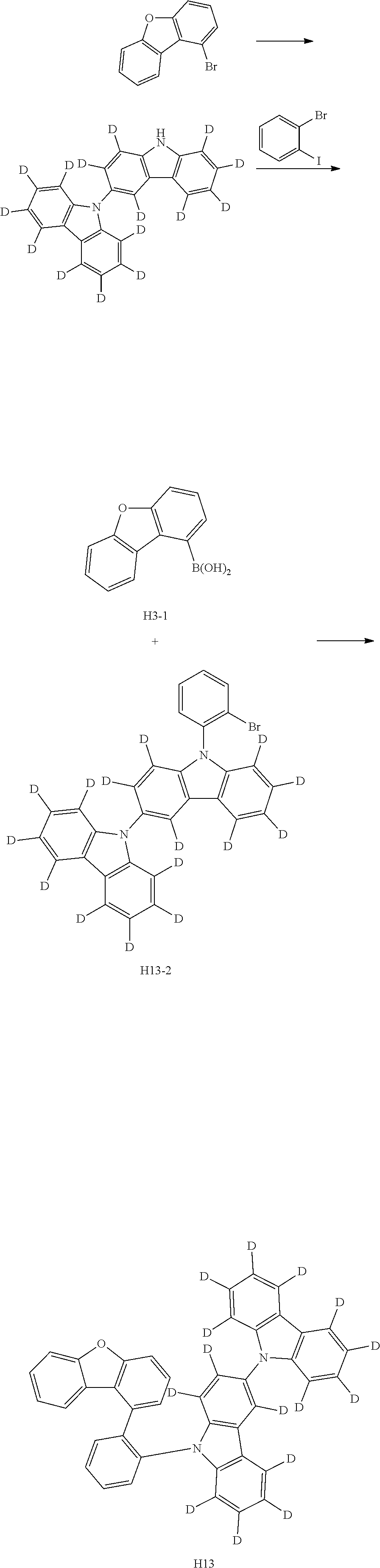

### (1) Synthesis of Intermediate H13-2

9H-3,9′-bicarbazole-1,1′, 2,2′, 3′, 4,4′, 5,5′, 6,6′, 7,7′, 8,8′-d15 (1 eq), 1-bromo-2-iodobenzene (1.5 eq), and K₃PO₄ (2 eq) were dissolved in DMF, followed by stirring overnight at 160° C. to obtain Intermediate H13-2. Intermediate H13-2 was identified by LC-MS as follows:

C₃₀H₄D₁₅BrN₂ M+1: 502.2.

### (2) Synthesis of Compound H13

1.4 g of Intermediate H3-1, 2.8 g of Intermediate H13-2, 0.3 g of tetrakis(triphenylphosphine)palladium, and 2 g of potassium carbonate were added to a reaction vessel, and dissolved in 100 mL of toluene, 20 mL of ethanol, and 20 mL of distilled water. The mixed solution was then refluxed for 24 hours. After the reaction was completed, the reaction solution was extracted with ethyl acetate, the collected organic layer was dried with magnesium sulfate and a solvent was evaporated therefrom. The obtained residue was separated and purified by silica gel column chromatography to obtain 3.1 g (yield: 58%) of Compound H13. Compound H13 was identified by LC-MS, and shown in Table 1.

### Synthesis Example 4: Synthesis of Compound H14

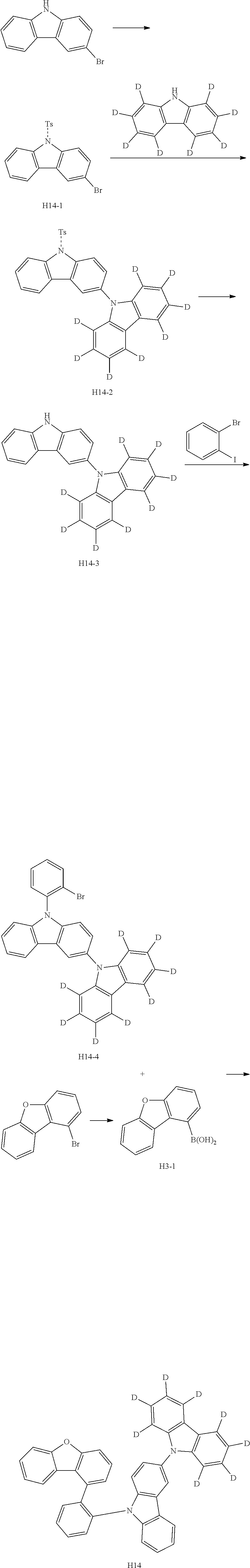

### (1) Synthesis of Intermediate H14-1

3-bromo-9H-carbazole (CAS #=1 592-95-6) (1 eq), TsCI (1 eq), and KOH (1 eq) were dissolved in acetone and then refluxed overnight to obtain Intermediate H14-1. Intermediate H14-1 was identified by LC-MS as follows:

C₁₉H₁₄BrNO₂SM+1:H400.1.

### (2) Synthesis of Intermediate H14-2

Intermediate H14-2 (1 eq) and 9H-carbazole-1,2,3,4,5,6,7,8-d8 (CAS #=38537-24-5) (1 eq) were dissolved in toluene and then refluxed overnight in the presence of CuI (0.5 eq), ethylenediamine (2 eq), and potassium phosphate (3 eq) to obtain Intermediate H14-2. Intermediate H14-2 was identified by LC-MS as follows:

C₃₁H₁₄D₈N₂O₂S M+1: 495.3.

### (3) Synthesis of Intermediate H14-3

Intermediate H14-2 (1 eq) and KOH (5 eq) were dissolved in a solution (THF:H2O=1:1) and then refluxed overnight to obtain Intermediate H14-3. Intermediate H14-3 was identified by LC-MS as follows:

C₂₄H₈D₈N₂ M+1 341.2.

### (4) Synthesis of Intermediate H14-4

Intermediate H14-3 (1 eq), 1-Bromo-2-iodobenzene (1.5 eq), and K₃PO₄ (2 eq) were dissolved in DMF, followed by stirring overnight at 160° C. to obtain Intermediate H14-4. Intermediate H14-4 was identified by LC-MS as follows:

C₃₀H₁₁D₈BrN₂ M+1 495.2.

### (5) Synthesis of Compound H14

1.3 g of Intermediate H3-1, 2.4 g of Intermediate H14-4, 0.2 g of tetrakis(triphenylphosphine)palladium, and 2 g of potassium carbonate were added to a reaction vessel, and dissolved in 100 mL of toluene, 20 mL of ethanol, and 20 mL of distilled water. The mixed solution was then refluxed for 24 hours. After the reaction was completed, the reaction solution was extracted with ethyl acetate, the collected organic layer was dried with magnesium sulfate and a solvent was evaporated therefrom. The obtained residue was separated and purified by silica gel column chromatography to obtain 2.7 g (yield: 57%) of Compound H14. Compound H14 was identified by LC-MS, and shown in Table 1.

### Synthesis Example 5: Synthesis of Compound E1

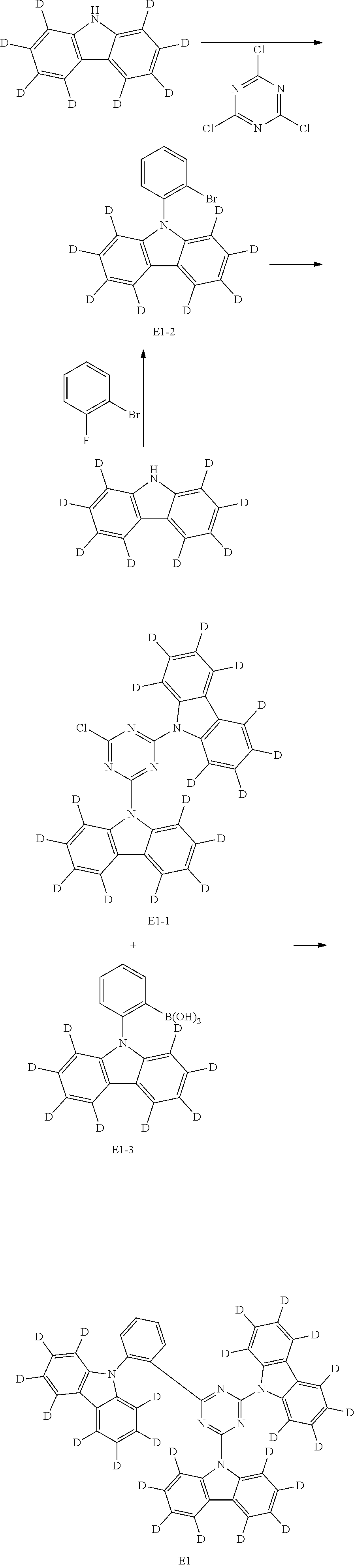

### (1) Synthesis of Intermediate E1-1

9H-carbazole-1,2,3,4,5,6,7,8-d8 (2 eq) (CAS #=38537-24-5) was dissolved in THE and contacted with n-butyllithium at 0° C. Then, cyanuric chloride was added dropwise thereto. The mixture was stirred overnight at 70° C. to obtain Intermediate E1-1. Intermediate E1-1 was identified by LC-MS as follows:

C₂₇D₁₆ClN₅ M+1 462.3.

### (2) Synthesis of Intermediate E1-2

9H-carbazole-1,2,3,4,5,6,7,8-d (1 eq), 1-bromo-2-fluorobenzene (1.5 eq), and K₃PO₄ (2 eq) were dissolved in DMF, followed by stirring overnight at 160° C. to obtain Intermediate E1-2. Intermediate E1-2 was identified by LC-MS as follows:

C₁₈H₄D₈BrN M+1: 330.2.

### (3) Synthesis of Intermediate E1-3

Intermediate E1-2 (1 eq) was dissolved in THE and then contacted with n-butyllithium (1.2 eq) at −78° C. After 1 hour, trimethyl borate was added dropwise thereto. Intermediate E1-3 was obtained by slowly raising the temperature to room temperature. Intermediate E1-3 was identified by LC-MS as follows:

C₁₈H₆D₈BNO₂ M+1 296.2.

### (4) Synthesis of Compound E1

2.1 g of Intermediate E1-1, 1.6 g of Intermediate E1-3, 0.21 g of tetrakis(triphenylphosphine)palladium, and 1.6 g of potassium carbonate were added to a reaction vessel, and dissolved in 40 mL of toluene, 10 mL of ethanol, and 10 mL of distilled water. The mixed solution was then refluxed for 24 hours. After the reaction was completed, the reaction solution was extracted with ethyl acetate, the collected organic layer was dried with magnesium sulfate and a solvent was evaporated therefrom. The obtained residue was separated and purified by silica gel column chromatography to obtain 1.6 g (yield: 59%) of Compound E1. Compound E1 was identified by LC-MS, and shown in Table 1.

### Synthesis Example 6: Synthesis of Compound E3

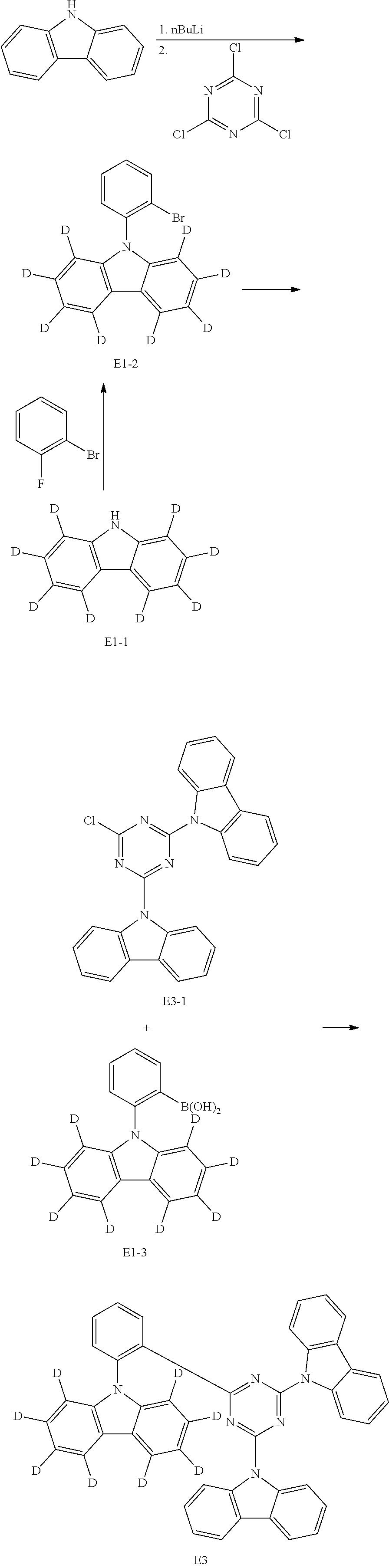

### (1) Synthesis of Intermediate E3-1

Carbazole (2 eq) (CAS #=86-74-8) was dissolved in THF and contacted with n-butyllithium at 0° C. Then, cyanuric chloride was added dropwise thereto. The mixture was stirred overnight at 70° C. to obtain Intermediate E3-1. Intermediate E3-1 was identified by LC-MS as follows:

C₂₇H₁₆ClN₅ M+1 446.1.

### (2) Synthesis of Compound E31

2.1 g of Intermediate E3-1, 1.6 g of Intermediate E13, 0.21 g of tetrakis(triphenylphosphine)palladium, and 1.6 g of potassium carbonate were added to a reaction vessel, and dissolved in 40 mL of toluene, 10 mL of ethanol, and 10 mL of distilled water. The mixed solution was then refluxed for 24 hours. After the reaction was completed, the reaction solution was extracted with ethyl acetate, the collected organic layer was dried with magnesium sulfate and a solvent was evaporated therefrom. The obtained residue was separated and purified by silica gel column chromatography to obtain 1.5 g (yield: 54%) of Compound E3. Compound E3 was identified by LC-MS, and shown in Table 1.

### Synthesis Example 7: Synthesis of Compound E4

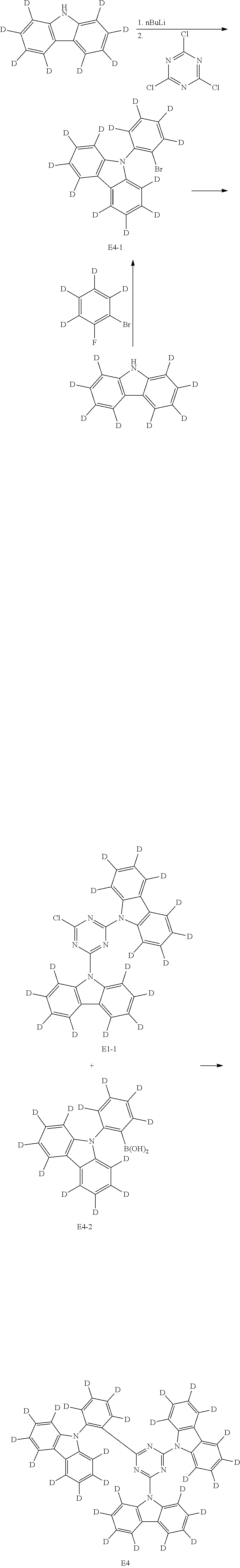

### (1) Synthesis of Intermediate E4-1

9H-carbazole-1,2,3,4,5,6,7,8-d (1 eq), 1-bromo-2-fluorobenzene-3,4,5,6-d4 (1.5 eq) (CAS #=50592-35-3), and K₃PO₄ (2 eq) were dissolved in DMF, followed by stirring overnight at 160° C. to obtain Intermediate E4-1. Intermediate E4-1 was identified by LC-MS as follows:

C₁₈D₁₂BrN M+1: 334.0.

### (2) Synthesis of Intermediate E4-2

Intermediate E4-1 (1 eq) was dissolved in THE and then contacted with n-butyllithium (1.2 eq) at −78° C. After 1 hour, trimethyl borate (1.4 eq) was added dropwise thereto. Intermediate E4-2 was obtained by slowly raising the temperature to room temperature. Intermediate E4-2 was identified by LC-MS as follows:

C₁₈H₂D₁₂BNO₂ M+1: 301.2.

### (3) Synthesis of Compound E4

3.7 g of Intermediate E1-1, 2.9 g of Intermediate E4-2, 0.37 g of tetrakis(triphenylphosphine)palladium, and 2.8 g of potassium carbonate were added to a reaction vessel, and dissolved in 80 mL of toluene, 20 mL of ethanol, and 20 mL of distilled water. The mixed solution was then refluxed for 24 hours. After the reaction was completed, the reaction solution was extracted with ethyl acetate, the collected organic layer was dried with magnesium sulfate and a solvent was evaporated therefrom. The obtained residue was separated and purified by silica gel column chromatography to obtain 3.3 g (yield: 61%) of Compound E4. Compound E4 was identified by LC-MS, and shown in Table 1.

For the compounds synthesized in Synthesis Examples 1 to 7 above, ¹H NMR and high-resolution mass (HR-MS) were measured, and the results are shown in Table 1. Synthesis methods of other compounds in addition to the compounds synthesized in Synthesis Examples 1 to 7 may be easily recognized by those skilled in the art by referring to the synthesis paths and source materials.

TABLE 1

| | | Molecular weight | Molecular weight |
|---|---|---|---|
| Com- | | (theoretical | (experimental value) |
| pound | Molecular formula | value) | [M + 1] |
| H1 | C₄₂D₂₆N₂O | 600.4 | 601.3 |
| H3 | C₄₂H₁₁D₁₅N₂O | 589.3 | 590.4 |
| H13 | C₄₂H₁₁D₁₅N₂O | 589.3 | 590.3 |
| H14 | C₄₂H₁₈D₈N₂O | 582.25 | 583.3 |
| E1 | C₄₅H₄D₂4N₂₆ | 676.39 | 677.4 |
| E3 | C₄₅H₂₀D₈N₆ | 660.29 | 661.3 |
| E4 | C₄₅D₂₈N₆ | 680.41 | 681.4 |

### Evaluation Example 1

The HOMO energy level, the LUMO energy level, and the triplet (Ti) energy level of each of Compounds H1, H3, H13, H14, E1, E3, E4, SH1 to SH3, SF1, SF2, and SF4 were measured by conducting quantum chemical calculations utilizing Gaussian 09, which is a quantum chemical calculation program manufactured to Gaussian Inc., U.S.A., and the results thereof are shown in Table 2. B3LYP was utilized as a structural optimization in a ground state, and 6-31G* (d,p) was utilized as a function.

TABLE 2

| Compound No. | HOMO (eV) | LUMO (eV) | T₁ (eV) |
|---|---|---|---|
| H1 | −5.42 | −1.48 | 3.05 |
| H3 | −5.43 | −1.45 | 3.06 |
| H13 | −5.38 | −1.57 | 2.91 |
| H14 | −5.38 | −1.57 | 2.91 |
| E1 | −5.61 | −2 | 2.77 |
| E3 | −5.65 | −2.27 | 2.74 |
| E4 | −5.61 | −2 | 2.77 |
| SH1 | −5.44 | −1.48 | 3.04 |
| SH2 | −5.39 | −1.63 | 3.08 |
| SH3 | −5.41 | −1.66 | 2.84 |
| SE1 | −5.61 | −2 | 2.77 |
| SE2 | 6.01 | −2.03 | 3.05 |
| SE4 | −5.51 | −1.79 | 2.77 |

### Evaluation Example 2

The phase transition temperature of each of Compounds H1, H3, H13, H14, E1, E3, E4 and SH1 to SH3, SE1, SE2, and SE4 was measured, and the results thereof are shown in Table 3. More specifically, each compound was first heated at an initial temperature of 100° C. under the pressure of 3.5*10-3 torr, and a temperature at which a phase transition occurred was measured.

TABLE 3

| Compound No. | Phase transition temperature (° C.) |
|---|---|
| H1 | 295 |
| H3 | 295 |
| H13 | 294 |
| H14 | 294 |
| E1 | 296 |
| E3 | 296 |
| E4 | 297 |
| SH1 | 295 |
| SH2 | 286 |
| SH3 | 296 |
| SE1 | 294 |
| SE2 | 334 |
| SE4 | 295 |

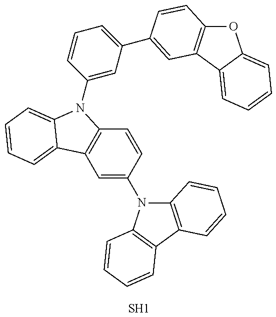

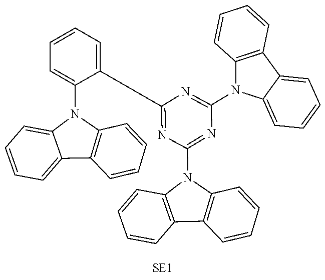 [SMILES: c1ccc(-n2c3ccccc3c3ccccc32)c(-c2nc(-n3c4ccccc4c4ccccc43)nc(-n3c4ccccc4c4ccccc43)n2)c1]

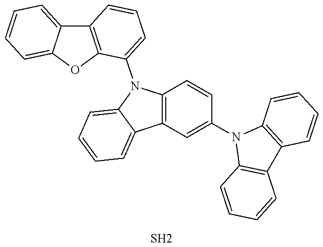

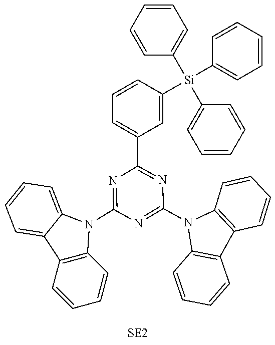 [SMILES: c1ccc([Si](c2ccccc2)(c2ccccc2)c2cccc(-c3nc(-n4c5ccccc5c5ccccc54)nc(-n4c5ccccc5c5ccccc54)n3)c2)cc1]

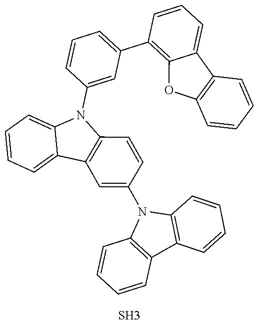

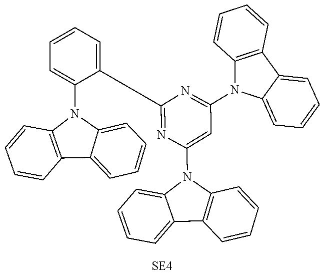 [SMILES: c1ccc(-n2c3ccccc3c3ccccc32)c(-c2nc(-n3c4ccccc4c4ccccc43)cc(-n3c4ccccc4c4ccccc43)n2)c1]

### Evaluation Example 3

1 g of Compound H1 and 0.54 g of Compound E1 were mixed (at a weight ratio of 65:35) and then triturated in a mortar to obtain a pre-mixed mixture. After filling the pre-mixed mixture in a crucible, a process of depositing the pre-mixed mixture on a glass substrate to have a thickness of 2,000 angstrom (Å) was repeated at a speed of 2 angstroms per second (Å/s) in a vacuum chamber until the pre-mixed mixture was exhausted. The obtained deposition layers 1 to 5 were each dissolved in dichloromethane, and the organic solvent was evaporated. HPLC analysis was conducted to identify changes in ratios among the compounds, and the results thereof are shown in Table 4.

TABLE 4

| Deposition layer | H1 | E1 |
|---|---|---|
| (2,000 Å) | (%) | (%) |
| 1 | 66.4 | 33.6 |
| 2 | 66.7 | 33.3 |
| 3 | 66.1 | 33.9 |
| 4 | 65.4 | 34.6 |
| 5 | 65.1 | 34.9 |

From Table 4, it is confirmed that a difference between the initial composition ratio of the pre-mixed mixture and the composition ratio of the obtained deposition layer is within 2%, and thus, the composition ratio does not substantially change during the deposition process. This suggests that the deposition process is stably performed.

### Example 1

As an anode, a glass substrate (product of Corning Inc.) with a 15 Ω/cm² (1,200 Å) ITO formed thereon was cut to a size of 50 mm×50 mm×0.5 mm, sonicated by utilizing isopropyl alcohol and pure water each for 5 minutes, cleaned by irradiation of ultraviolet rays and exposure of ozone thereto for 30 minutes, and then mounted on a vacuum deposition apparatus.

HATCN was vacuum-deposited on the anode to form a hole injection layer having a thickness of 100 Å, BCFN was vacuum-deposited on the hole injection layer to from a first hole transport layer having a thickness of 600 Å, and SiCzCz was vacuum-deposited on the first hole transport layer to form a second hole transport layer having a thickness of 50 Å.

P1 (host) as a pre-mixed mixture of Compounds H1 and E1, and PtON-TBBI (phosphorescent dopant), were concurrently (e.g., simultaneously) vacuum-deposited on the hole transport layer to from an emission layer having a thickness of 350 Å. The weight ratio of P1 and PtON-TBBI was adjusted to 87:13.

mSiTrz was vacuum-deposited on the emission layer to from a first electron transport layer having a thickness of 50 Å, mSiTrz and LiQ were concurrently (e.g., simultaneously) vacuum-deposited on the first electron transport layer at a weight ratio of 1:1 to form a second electron transport layer having a thickness of 350 Å, LiF was vacuum-deposited on the second electron transport layer to from an electron injection layer having a thickness of 15 Å, and Al was vacuum-deposited on the electron injection layer to form a cathode having a thickness of 80 Å, thereby manufacturing an organic light-emitting device.

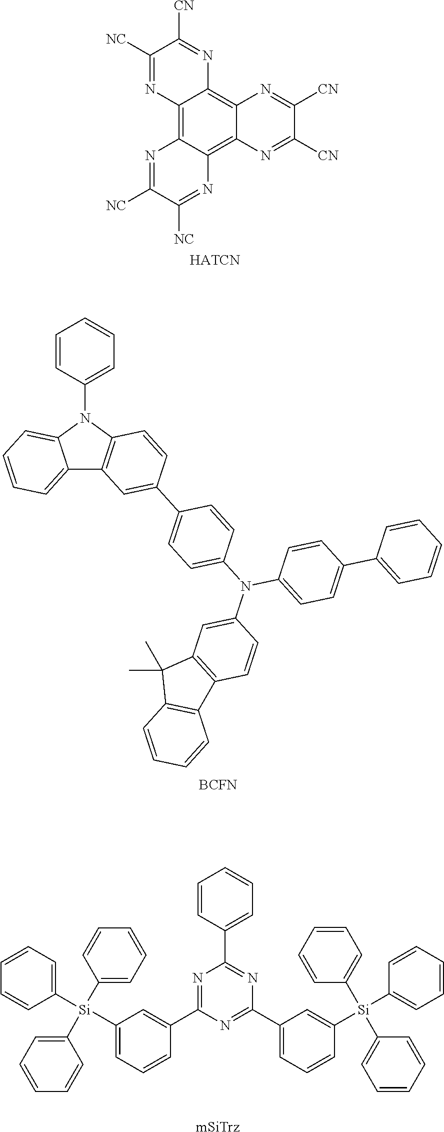

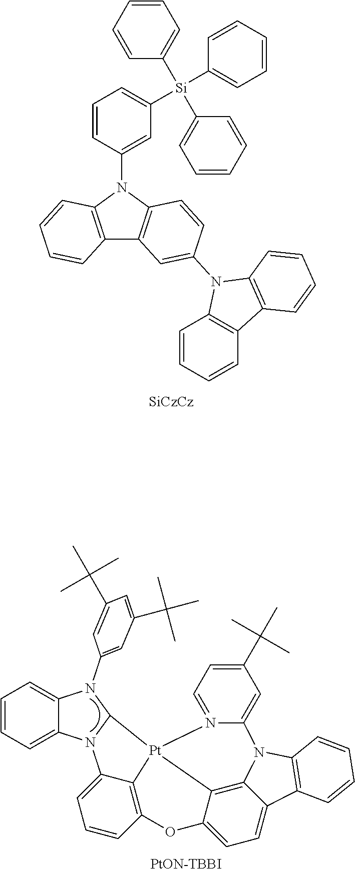

### Examples 2 to 7 and Comparative Examples 1 to 4

The organic light-emitting devices of Examples 2 to 7 and Comparative Examples 1 to 4 (CE-1 to CE-4) were manufactured in substantially the same manner as in Example 1, except that in forming an emission layer, the pre-mixed mixture P1 was changed as shown in Table 5.

TABLE 5

| | Pre-mixed | First | Second |
|---|---|---|---|
| | mixture No. | compound | compound |
| Example 1 | P1 | H1 | E1 |
| Example 2 | P2 | H3 | E1 |
| Example 3 | P3 | H1 | E4 |
| Example 4 | P4 | H3 | E3 |
| Example 5 | P5 | H14 | E3 |
| Example 6 | P6 | H13 | E3 |
| CE-1 | S1 | SH1 | SE1 |
| CE-2 | S2 | SH2 | SE2 |
| CE-3 | S3 | SH3 | — |
| CE-4 | S4 | — | SE4 |

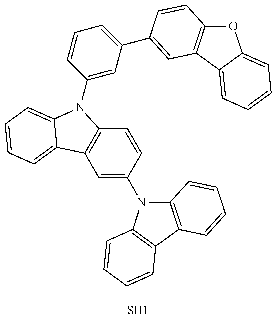

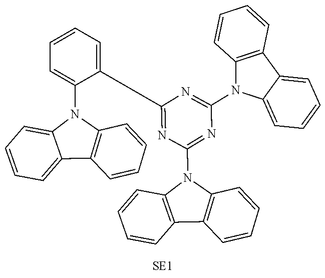 [SMILES: c1ccc(-n2c3ccccc3c3ccccc32)c(-c2nc(-n3c4ccccc4c4ccccc43)nc(-n3c4ccccc4c4ccccc43)n2)c1]

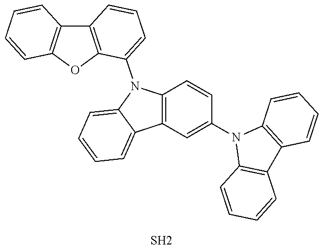

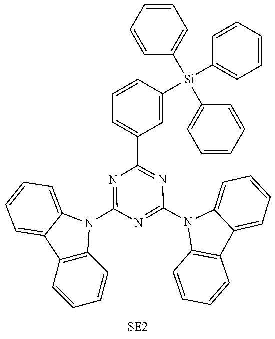 [SMILES: c1ccc([Si](c2ccccc2)(c2ccccc2)c2cccc(-c3nc(-n4c5ccccc5c5ccccc54)nc(-n4c5ccccc5c5ccccc54)n3)c2)cc1]

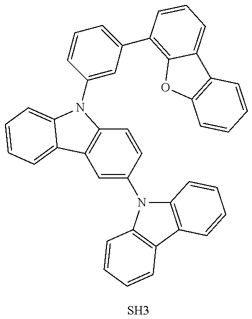

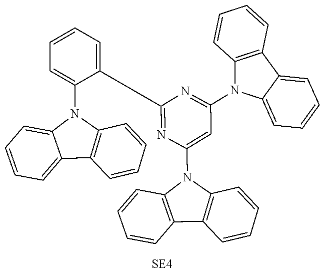 [SMILES: c1ccc(-n2c3ccccc3c3ccccc32)c(-c2nc(-n3c4ccccc4c4ccccc43)cc(-n3c4ccccc4c4ccccc43)n2)c1]

### Evaluation Example 4

The driving voltage (V) at the current density of 10 milliampere per square centimeter (mA/cm²), the maximum quantum efficiency (%), the device relative lifespan (%), and the emission color of the organic light-emitting devices manufactured in Examples 1 to 6 and Comparative Examples 1 to 4 (CE-1 to CE-4) were measured and shown in Table 6. In Table 6, the driving voltage was measured utilizing a source meter (Keithley Instrument Inc., 2400 series), and the maximum external quantum efficiency was measured utilizing an external quantum efficiency measurement apparatus C9920-2-12 of Hamamatsu Photonics Inc. In evaluating the maximum quantum efficiency, the luminance/current density was measured utilizing a luminance meter that was calibrated for wavelength sensitivity, and the maximum quantum efficiency was converted by assuming an angular luminance distribution (Lambertian) which introduces a perfect reflecting diffuser. The device relative lifespan is a relative lifespan measured based on the time (100%) taken for the luminance of Comparative Example 1 to reach 95% of the initial luminance.

TABLE 6

| | | Driving | Maximum | Device | |
|---|---|---|---|---|---|
| | Host | voltage | quantum | relative | Emission |
| Device ID | material | (V) | efficiency (%) | lifespan (%) | color |
| Example 1 | P1 | 4.8 | 21.8 | 117 | Blue |
| Example 2 | P2 | 4.8 | 20.8 | 108 | Blue |
| Example 3 | P3 | 4.8 | 22.0 | 121 | Blue |
| Example 4 | P4 | 4.8 | 20.8 | 103 | Blue |
| Example 5 | P5 | 5.1 | 20.3 | 101 | Blue |
| Example 6 | P6 | 5.2 | 20.4 | 103 | Blue |
| CE-1 | S₁ | 4.8 | 20.2 | 100 | Blue |
| CE-2 | S2 | 5.1 | 19.8 | 92 | Blue |
| CE-3 | S3 | 6.6 | 12.8 | 34 | Blue |
| CE-4 | S4 | 6.1 | 15.4 | 49 | Blue |

From Table 6, it was confirmed that the organic light-emitting devices of Examples 1 to 6 each emitted blue light and had better (e.g., decreased) driving voltage, more excellent or suitable luminescence efficiency (i.e., maximum quantum efficiency), and they also had longer lifespan characteristics compared to the organic light-emitting devices of Comparative Examples 1 to 4 (CE-1 to CE-4).

As the composition has excellent or suitable luminescence efficiency and lifespan characteristics as well as improved electric characteristics and durability, a light-emitting device employing the composition may also have excellent or suitable luminescence efficiency and lifespan characteristics and improved electric characteristics and durability.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that one or more suitable changes in form and details may be made therein without departing from the spirit and scope as defined by the following claims and equivalents thereof.

What is claimed is:

1. A composition comprising:
a first compound represented by Formula 1; and
a second compound represented by Formula 2: 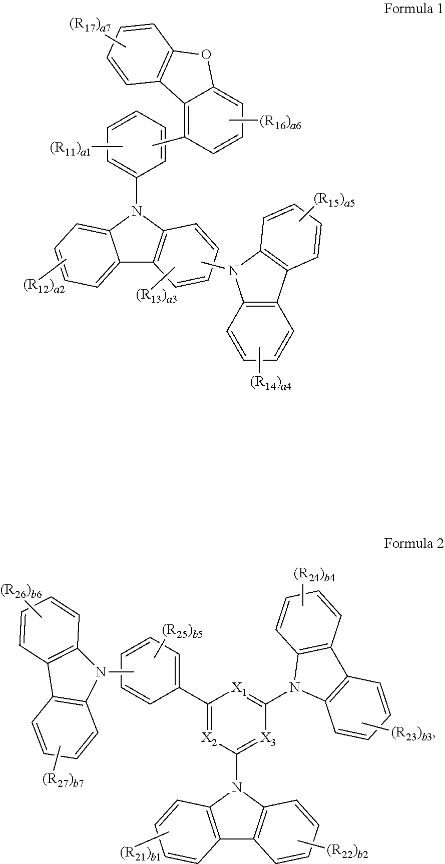
and
wherein, in Formulae 1 and 2,
R₁₁ to R₁₇ and R₂₁ to R₂₇ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),
a1 to a7 and b1 to b7 are each independently an integer from 1 to 4,
X₁ is N or C(Y₁),
X₂ is N or C(Y₂),
X₃ is N or C(Y₃),
at least one of selected from among X₁ to X₃ is N,
Y₁ to Y₃ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), -B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to 033 are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or any combination thereof.

2. The composition of claim 1, wherein the first compound comprises at least one deuterium,
the second compound comprises at least one deuterium, or
each of the first compound and the second compound comprises at least one deuterium.

3. The composition of claim 1, wherein a highest occupied molecular orbital (HOMO) energy level of the first compound is −5.6 eV or more.

4. The composition of claim 1, wherein a lowest unoccupied molecular orbital (LUMO) energy level of the second compound is −2.6 eV or less.

5. The composition of claim 1, wherein a triplet (T1) energy level of each of the first compound and the second compound is 2.8 eV or more.

6. The composition of claim 1, wherein a difference between a phase transition temperature of the first compound and a phase transition temperature of the second compound is 20° C. or less.

7. The composition of claim 1, wherein a phase transition temperature of the first compound is about 285° C. to about 305° C., and a phase transition temperature of the second compound is about 285° C. to about 305° C.

8. The composition of claim 1, wherein an amount of the first compound is about 1 part by weight to about 99 parts by weight based on 100 parts by weight of the composition, and
an amount of the second compound is about 1 part by weight to about 99 parts by weight based on 100 parts by weight of the composition.

9. A light-emitting device comprising:
a first electrode;
a second electrode facing the first electrode;
an interlayer between the first electrode and the second electrode and comprising an emission layer;
a first compound represented by Formula 1; and
a second compound represented by Formula 2: 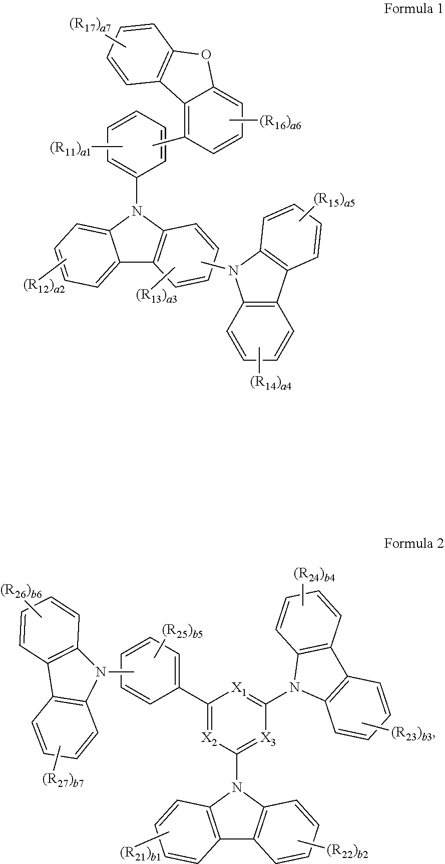
wherein, in Formulae 1 and 2,
R₁₁ to R₁₇ and R₂₁ to R₂₇ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),
a1 to a7 and b1 to b7 are each independently an integer from 1 to 4,
X₁ is N or C(Y₁),
X₂ is N or C(Y₂),
X₃ is N or C(Y₃),
at least one of selected from among X₁ to X₃ is N,
Y₁ to Y₃ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), -B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to 033 are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or any combination thereof.

10. The light-emitting device of claim 9, wherein the emission layer comprises the first compound and the second compound, and
the emission layer further comprises a transition metal-containing compound, a delayed fluorescence compound, or any combination thereof.

11. The light-emitting device of claim 10, wherein the transition metal-containing compound comprises platinum (Pt).

12. The light-emitting device of claim 10, wherein the transition metal-containing compound comprises platinum and a tetradentate ligand bonded to the platinum, and
the platinum and one carbon atom of the tetradentate ligand are bonded to each other via a coordinate bond.

13. The light-emitting device of claim 10, wherein the delayed fluorescence compound is a compound comprising at least one cyclic group comprising each of boron (B) and nitrogen (N), as a ring forming atom.

14. The light-emitting device of claim 9, wherein a maximum emission wavelength of light emitted from the emission layer is about 400 nanometer (nm) to about 500 nm.

15. A method of manufacturing a light-emitting device, wherein the light-emitting device comprises:
a first electrode;
a second electrode facing the first electrode;
an interlayer between the first electrode and the second electrode and comprising an emission layer;
a first compound represented by Formula 1; and
a second compound represented by Formula 2,
the method comprising:
applying a composition-containing layer by filling a deposition source in a vacuum chamber with the composition of claim 1; and
performing a deposition process of heating the composition.

16. The method of claim 15, wherein a deposition temperature of the deposition process is about 180° C. to about 340° C.

17. An electronic apparatus comprising the light-emitting device of claim 9.

18. The electronic apparatus of claim 17, further comprising a thin-film transistor,
wherein the thin-film transistor comprises a source electrode and a drain electrode, and
the first electrode of the light-emitting device is electrically connected to at least one selected from among the source electrode and the drain electrode of the thin-film transistor.

19. An electronic equipment comprising the light-emitting device of claim 9.

20. The electronic equipment of claim 19, wherein the electronic equipment is a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, and/or a signboard.
